(19) United States
(12) Patent Application Publication
(10) Pub. No.: US 2026/0168028 A1
(43) Pub. Date: Jun. 18, 2026

(54) Methods and Systems for Diagnosis and Treatment of Lupus Based on Expression of Primary Immunodeficiency Genes

(71) Applicant: AMPEL BioSolutions, LLC, Charlottesville, VA (US)

(72) Inventors: Haley DAVIS, Charlottesville, VA (US); Adam C. LABONTE, Charlottesville, VA (US); Katherine A. OWEN, Charlottesville, VA (US); Prathyusha BACHALI, Charlottesville, VA (US); Amrie C. GRAMMER, Charlottesville, VA (US); Peter E. LIPSKY, Charlottesville, VA (US)

(21) Appl. No.: 19/199,677

(22) Filed: May 6, 2025

Related U.S. Application Data
(63) Continuation of application No. PCT/US2023/032946, filed on Sep. 15, 2023.
(60) Provisional application No. 63/423,753, filed on Nov. 8, 2022.

(51) Int. Cl. C12Q 1/6883; G16B 20/20; G16B 25/10; G16B 40/00; G16H 50/20

(52) U.S. Cl. CPC C12Q 1/6883; G16B 20/20; G16B 25/10; G16B 40/00; G16H 50/20; C12Q 2600/156; C12Q 2600/158

(57) ABSTRACT

Methods for classifying a lupus disease state of a patient is provided. The method can include analyzing a data set comprising or derived from gene expression measurements of at least 2 genes selected from genes listed in Table 3, and Tables 5-1 to 5-20 to classify the lupus disease state of the patient, wherein the gene expression measurements are obtained from a biological sample of the patient.

### CROSS-REFERENCE

This application is a continuation of PCT/US2023/032946, filed on Sep. 15, 2023, which claims the benefit of U.S. Provisional Patent Application No. 63/423,753, filed on Nov. 8, 2022, the contents of which are incorporated herein by reference in their entirety.

### BACKGROUND

Lupus, including Systemic Lupus Erythematosus (SLE), is heterogeneous in nature and has variable causation, course and responsiveness to therapy. Genetics plays a role in both SLE susceptibility and severity, however genetic loci contributing to SLE disease pathogenesis remains poorly understood. There is a need for understanding risk loci involved in the pathogenesis of these conditions to allow identification and optimization of therapies.

### SUMMARY

One aspect of the present disclosure is directed to a method for classifying the lupus disease state of a patient. In certain aspects, the method can include analyzing a data set comprising or derived from gene expression measurements of at least 2 genes to classify the lupus disease state of the patient. In certain embodiments, the data set comprises or is derived from gene expression measurements of at least 2 genes selected from genes listed in Table 3, and Tables 5-1 to 5-20. In certain embodiments, data set comprises or is derived from gene expression measurements of at least 2 genes selected from genes listed in Table 3. In certain embodiments, the data set comprises or is derived from gene expression measurements of at least 2 genes selected from genes listed in Tables 5-1 to 5-20. In certain embodiments, the data set comprises or is derived from gene expression measurements of at least 2 genes selected from genes listed in Tables 5-1 to 5-4, 5-6 to 5-10, and 5-12 to 5-20. The at least 2 genes may or may not include gene(s) that are not listed within the genes listed in Table 3, and Tables 5-1 to 5-20. In certain embodiments, the at least 2 genes do not include any gene that are not listed within the genes listed in Tables 5-1 to 5-20. In certain embodiments, the at least 2 genes do not include any gene that is not listed within the genes listed in Tables 5-1 to 5-4, 5-6 to 5-10, and 5-12 to 5-20. In certain embodiments, classifying the lupus disease state of the patient includes classifying (e.g., determining) whether the patient has lupus, wherein the dataset is analyzed to classify whether the patient has lupus. In certain embodiments, classifying the lupus disease state of the patient includes classifying (e.g., determining) whether the patient has active lupus or inactive lupus, wherein the dataset is analyzed to classify whether the patient has active lupus or inactive lupus. In certain embodiments, classifying the lupus disease state of the patient includes classifying (e.g., determining) whether the patient has active lupus, inactive lupus or does not have lupus, wherein the dataset is analyzed to classify whether the patient has active lupus, inactive lupus or does not have lupus. The gene expression measurements can be obtained from a biological sample obtained or derived from the patient. The lupus disease state of the patient can be classified based on expression of the at least 2 genes in the biological sample.

In certain embodiments, the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450 or 453 genes selected from the genes listed in Table 3, and Tables 5-1 to 5-20, from the biological sample from the patient. In certain embodiments, genes of the data set, e.g., gene expression measurement of which the data set is comprised of or derived from, are selected from the genes listed in Table 3. In certain embodiments, genes of the data set, are selected from the genes listed in Tables 5-1 to 5-20. In certain embodiments, genes of the data set, are selected from the genes listed in Tables 5-1 to 5-4, 5-6 to 5-10, and 5-12 to 5-20. In certain embodiments, the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 445 or 447 genes selected from the genes listed in Tables 5-1 to 5-4, 5-6 to 5-10, and 5-12 to 5-20 from the biological sample from the patient.

In certain embodiments, the data set comprises or is derived from gene expression measurements of at least 2 genes selected from the genes listed in each of one or more Tables selected from Tables 5-1 to 5-4, 5-6 to 5-10, and 5-12 to 5-20, wherein number of genes selected from the each selected Tables can be the same or different. As a non-limiting example, if 3 Tables, such as Table 5-1, 5-2 and 5-3, are selected from Tables 5-1 to 5-4, 5-6 to 5-10, and 5-12 to 5-20, the data set comprises or is derived from gene expression measurements of at least 2 genes selected from the genes listed in each of the 3 selected Tables, e.g., the data set comprises or is derived from gene expression measurements of at least 2 genes selected from the genes listed in Table 5-1, at least 2 genes selected from the genes listed in Table 5-2, and at least 2 genes selected from the genes listed in Table 5-3. In certain embodiments, the data set comprises or is derived from gene expression measurements of an effective number of genes selected from the genes listed in each of one or more Tables selected from Tables 5-1 to 5-4, 5-6 to 5-10, and 5-12 to 5-20, wherein number of genes selected from the each selected Tables can be the same or different. In certain embodiments, the data set comprises or is derived from gene expression measurements of the genes listed in each of one or more Tables selected from Tables 5-1 to 5-4, 5-6 to 5-10, and 5-12 to 5-20. In certain embodiments, the one or more Tables comprises 1, 2, 3, 4, 5, 6, 7, 8, 9, 10, 11, 12, 13, 14, 15, 16, 17 or 18, or any range there between Tables. In certain embodiments, the one or more Tables comprises 18 Tables, i.e, Tables 5-1 to 5-4, 5-6 to 5-10, and 5-12 to 5-20, are selected.

In certain embodiments, the data set comprises an enrichment score derived from the gene expression measurements, and the enrichment score is analyzed to classify the lupus disease state of the patient. In certain embodiments, the enrichment score is derived from the gene expression measurements using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof. In certain embodiments, the enrichment score is derived from the gene expression measurements using GSVA. In certain embodiments, the data set is derived from the gene expression measurements data using GSVA, and the data set comprises one or more GSVA scores of the patient. The one or more GSVA scores of the patient can be analyzed to classify the lupus disease state of the patient. The one or more GSVA scores of the patient can be generated based on the one or more Tables selected from Tables 5-1 to 5-4, 5-6 to 5-10, and 5-12 to 5-20. For each selected Table, at least one GSVA score of the patient is generated based on enrichment of expression of at least 2 genes selected from the genes listed in the selected Table, in the biological sample. In certain embodiments, for each selected Table at least one GSVA score of the patient is generated based on enrichment of expression of an effective number of genes selected from the genes listed in the selected Table, in the biological sample, wherein genes selected from different selected Tables can be the same or different. In certain embodiments, for each selected Table at least one GSVA score of the patient is generated based on enrichment of expression of the genes listed in the selected Table, in the biological sample. For respective selected table, the genes selected (e.g., at least 2 genes, effective number of genes, or all genes) from the Table can form the input gene set for generating the at least one GSVA score based on the respective selected Table, using GSVA. The one or more GSVA scores can contain the generated GSVA scores. In certain embodiments, for each selected Table one GSVA score is generated, as a non-limiting example if 3 Tables, such as Tables 5-1, 5-2 and 5-3 are selected, the one or more GSVA scores contain 3 GSVA scores, wherein 1 GSVA score generated based on Table 5-1, 1 GSVA generated based on Table 5-2, and 1 GSVA generated based on Table 5-3, wherein the GSVA score based on Table 5-1 is generated based on enrichment of the genes selected (e.g., at least 2 genes, effective number of genes, or all genes) from the Table 5-1, in the biological sample; the GSVA score based on Table 5-2 is generated based on enrichment of the genes selected from the Table 5-2, in the biological sample; and the GSVA score based on Table 5-3 is generated based on enrichment of the genes selected from the Table 5-3, in the biological sample. The one or more GSVA scores of the patient can be generated based on comparing the gene expression measurements from the biological sample with a reference dataset. The reference dataset can be a reference dataset as described herein. The one or more GSVA scores of the patient can be generated using the input gene sets using a method described in the Examples, and/or as understood by a person of ordinary skill in the art.

In certain embodiments, analyzing the data set comprises providing the data set as an input to a machine-learning model, wherein the machine learning model generates an inference indicative of the lupus disease state of the patient, based on the data set. The method can classify the lupus disease state of the patient based on the inference. In certain embodiments, the method further comprises: receiving, as an output of the machine-learning model, the inference indicative of the lupus disease state of the patient; and/or electronically outputting a report classifying the lupus disease state of the patient, based on the inference.

The machine learning model can be trained using linear regression, logistic regression (LOG), Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, a Random Forest (RF), deep learning algorithm, linear discriminant analysis (LDA), decision tree learning (DTREE), adaptive boosting (ADB), Classification and Regression Tree (CART), hierarchical clustering, or any combination thereof.

The inference can include a confidence value between 0 and 1. In certain embodiments, the confidence value of the inference is between 0 and 1, that the patient has lupus. In certain embodiments, the confidence value of the inference is between 0 and 1, that the patient has active lupus. In certain embodiments, the confidence value of the inference is between 0 and 1, that the patient has inactive lupus.

In certain embodiments, the lupus disease state of the patient is classified with an accuracy of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In certain embodiments, the lupus disease state of the patient is classified with a sensitivity of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In certain embodiments, the lupus disease state of the patient is classified with a specificity of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In certain embodiments, the lupus disease state of the patient is classified with a positive predictive value of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In certain embodiments, the lupus disease state of the patient is classified with a negative predictive value of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The machine learning model can have a receiver operating characteristic (ROC) curve with an Area-Under-Curve (AUC) of at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

In certain embodiments, analyzing the data set comprises developing a risk score for the patient based at least on the data set, and classifying the lupus disease state of the patient based at least on the risk score of the patient. In certain embodiments, the risk score for the patient is developed based on the enrichment score, such as one or more GSVA scores, of the patient.

The biological sample can comprises a blood sample, isolated peripheral blood mononuclear cells (PBMCs), tissue biopsy sample, nasal fluid, saliva, urine, stool, or any derivative thereof. In certain embodiments, the biological sample comprises a blood sample, isolated peripheral blood mononuclear cells (PBMCs), or any derivative thereof.

In certain embodiments, the at least 2 genes are selected from genes listed in Table 5-16, Table 5-15, Table 5-18, and Table 5-10, and the data set is analyzed to classify whether the patient has lupus. In certain embodiments, the data set comprises or is derived from gene expression measurements of at least 2 to all, or any value or range there between genes selected from genes listed in Table 5-16, Table 5-15, Table 5-18, and Table 5-10, and the data set is analyzed to classify whether the patient has lupus. In certain embodiments, the data set comprises or is derived from gene expression measurements of at least 2 genes selected from the genes listed in each of 2, 3, or 4 Tables selected from Table 5-16, Table 5-15, Table 5-18, and Table 5-10, and the data set is analyzed to classify whether the patient has lupus. In certain embodiments, the data set comprises or is derived from gene expression measurements of at least 2 genes selected from the genes listed in each of Table 5-16, Table 5-15, Table 5-18, and Table 5-10, (i.e., the data set comprises or is derived from gene expression measurements of at least 2 genes selected from the genes listed in Table 5-16, at least 2 genes selected from the genes listed in Table 5-15, at least 2 genes selected from the genes listed in Table 5-18, and at least 2 genes selected from the genes listed in Table 5-10), and the data set is analyzed to classify whether the patient has lupus. In certain embodiments, the data set comprises or is derived from gene expression measurements of an effective number of genes selected from the genes listed in each of 2, 3, or 4 Tables selected from Table 5-16, Table 5-15, Table 5-18, and Table 5-10, and the data set is analyzed to classify whether the patient has lupus. In certain embodiments, the data set comprises or is derived from gene expression measurements of an effective number of genes selected from the genes listed in each of Table 5-16, Table 5-15, Table 5-18, and Table 5-10, and the data set is analyzed to classify whether the patient has lupus. In certain embodiments, the data set comprises or is derived from gene expression measurements of the genes listed in each of 2, 3, or 4 Tables selected from Table 5-16, Table 5-15, Table 5-18, and Table 5-10, and the data set is analyzed to classify whether the patient has lupus. In certain embodiments, the one or more GSVA scores of the patient are generated based on 1, 2, 3 or 4 Tables selected from Table 5-16, Table 5-15, Table 5-18, and Table 5-10, and the data set is analyzed to classify whether the patient has lupus. In certain embodiments, the Table 5-16, Table 5-15, Table 5-18, and Table 5-10, are selected, and the one or more GSVA scores of the patient, comprises 4 GSVA scores, wherein one GVSA score is generated based on each selected Table, and the data set is analyzed to classify whether the patient has lupus.

In certain embodiments, the at least 2 genes are selected from genes listed in Table 5-16, Table 5-20, Table 5-19, Table 5-4, and Table 5-17, and the data set is analyzed to classify whether the patient has active lupus, or inactive lupus. In certain embodiments, the data set comprises or is derived from gene expression measurements of at least 2 to all, or any value or range there between genes selected from genes listed in Table 5-20, Table 5-19, Table 5-4, and Table 5-17, and the data set is analyzed to classify whether the patient has active lupus, or inactive lupus. In certain embodiments, the data set comprises or is derived from gene expression measurements of at least 2 genes selected from the genes listed in each of 2, 3, or 4 Tables selected from Table 5-20, Table 5-19, Table 5-4, and Table 5-17, and the data set is analyzed to classify whether the patient has active lupus, or inactive lupus. In certain embodiments, the data set comprises or is derived from gene expression measurements of at least 2 genes selected from the genes listed in each of Table 5-20, Table 5-19, Table 5-4, and Table 5-17, (i.e., the data set comprises or is derived from gene expression measurements of at least 2 genes selected from the genes listed in Table 5-20, at least 2 genes selected from the genes listed in Table 5-19, at least 2 genes selected from the genes listed in Table 5-4, and at least 2 genes selected from the genes listed in Table 5-17), and the data set is analyzed to classify whether the patient has active lupus, or inactive lupus. In certain embodiments, the data set comprises or is derived from gene expression measurements of an effective number of genes selected from the genes listed in each of 2, 3, or 4 Tables selected from Table 5-20, Table 5-19, Table 5-4, and Table 5-17, and the data set is analyzed to classify whether the patient has active lupus, or inactive lupus. In certain embodiments, the data set comprises or is derived from gene expression measurements of an effective number of genes selected from the genes listed in each of Table 5-20, Table 5-19, Table 5-4, and Table 5-17, and the data set is analyzed to classify whether the patient has active lupus, or inactive lupus. In certain embodiments, the data set comprises or is derived from gene expression measurements of the genes listed in each of 2, 3, or 4 Tables selected from Table 5-20, Table 5-19, Table 5-4, and Table 5-17, and the data set is analyzed to classify whether the patient has active lupus, or inactive lupus. In certain embodiments, the one or more GSVA scores of the patient are generated based on 1, 2, 3 or 4 Tables selected from Table 5-20, Table 5-19, Table 5-4, and Table 5-17, and the data set is analyzed to classify whether the patient has active lupus, or inactive lupus. In certain embodiments, the Table 5-20, Table 5-19, Table 5-4, and Table 5-17, are selected, and the one or more GSVA scores of the patient, comprises 4 GSVA scores, wherein one GVSA score is generated based on each selected Table, and the data set is analyzed to classify whether the patient has active lupus, or inactive lupus.

In certain embodiments, the method further comprises administering a treatment to the patient based on the classification of the lupus disease state of the patient. In certain embodiments, the treatment is configured to treat lupus. In certain embodiments, the treatment is configured to reduce severity of lupus. In certain embodiments, the treatment is configured to reduce a risk of having lupus. In certain embodiments, the treatment is configured to treat active lupus. In certain embodiments, the treatment is configured to reduce severity of active lupus. In certain embodiments, the treatment is configured to reduce a risk of having active lupus. In certain embodiments, the treatment is configured to treat inactive lupus. In certain embodiments, the treatment is configured to reduce severity of inactive lupus. In certain embodiments, the treatment is configured to reduce a risk of having inactive lupus.

In certain embodiments, the treatment for lupus comprises a neutrophil function inhibitor, a TNF inhibitor, an IL1 inhibitor, a Plasma cell inhibitor, a NK cell inhibitor, a B Cell Inhibitor, or any combination thereof. Non-limiting examples of an IFN inhibitor include Anifrolumab. Non-limiting examples of a Plasma cell inhibitor include Mycophenolate, Bortezomib, Carfilzomib, Ixazomib, Daratumumab, Isatuximab and Elotuzumab. Non-limiting examples of an IL1 inhibitor include Anakinra, and Canakinumab. Non-limiting examples of a TNF inhibitor include Adalimumab, Certolizumab pegol, Etanercept, Golimumab, and Infliximab. Non-limiting examples of a Neutrophil function inhibitor include Dasatinib, Apremilast, and Roflumilast. Non-limiting examples of a NK cell inhibitor include Azathioprine. Non-limiting examples of a B cell inhibitor include Belimumab, Rituximab, Obinutuzumab, and Inebilizumab. In certain embodiments, the treatment for lupus comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Inebilizumab, or any combination thereof.

The patient can be a human patient. In certain embodiments, the patient has lupus. In certain embodiments, the patient is asymptomatic of lupus. In certain embodiments, the patient is suspected of having lupus. In certain embodiments, the patient has active lupus. In certain embodiments, the patient is suspected of having active lupus. In certain embodiments, the patient has inactive lupus. In certain embodiments, the patient is suspected of having inactive lupus.

One aspect of the present disclosure is directed to a method for diagnosing lupus in a patient. The method comprises detecting presence of one or more single nucleotide polymorphisms (SNPs) selected from the SNPs listed in Table 3, in a biological sample from the patient. Detecting presence of the one or more SNPs, in a biological sample can include detecting whether or not the one or more SNPs are present in the biological sample. The patient is determined to have lupus, or is at risk of developing lupus when the one or more SNPs are present in the biological sample.

In certain embodiments, the method comprises detecting presence of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, or 137 SNPs selected from the SNPs listed in Table 3, are detected in the biological sample, and the patient is determined to have lupus, or is at risk of developing lupus when the SNPs are present in the biological sample.

The presence of the SNPs in the biological sample can be determined by analyzing a nucleic acid of the patient in the biological sample. In certain embodiments, analyzing the nucleic acid comprises sequencing at least a portion of DNA of the patient in the biological sample. In certain embodiments, analyzing the nucleic acid comprises analyzing expression of the genes associated with the one or more SNPs. In Table 3, for a respective SNP, the associated genes are listed in the same row.

The biological sample can comprises a blood sample, isolated peripheral blood mononuclear cells (PBMCs), tissue biopsy sample, nasal fluid, saliva, urine, stool, or any derivative thereof. In certain embodiments, the biological sample comprises a blood sample, isolated peripheral blood mononuclear cells (PBMCs), or any derivative thereof.

In certain embodiments, the method further comprises administering a treatment to the patient. The treatment can be administered based on the determination that the patient has lupus, or is at risk of developing lupus. In certain embodiments, the treatment is configured to treat lupus. In certain embodiments, the treatment is configured to reduce severity of lupus. In certain embodiments, the treatment is configured to reduce a risk of having lupus. In certain embodiments, the treatment is configured to treat active lupus. In certain embodiments, the treatment is configured to reduce severity of active lupus. In certain embodiments, the treatment is configured to reduce a risk of having active lupus. In certain embodiments, the treatment is configured to treat inactive lupus. In certain embodiments, the treatment is configured to reduce severity of inactive lupus. In certain embodiments, the treatment is configured to reduce a risk of having inactive lupus.

In certain embodiments, the treatment for lupus comprises a neutrophil function inhibitor, a TNF inhibitor, an IL1 inhibitor, a Plasma cell inhibitor, a NK cell inhibitor, a B Cell Inhibitor, or any combination thereof. Non-limiting examples of an IFN inhibitor include Anifrolumab. Non-limiting examples of a Plasma cell inhibitor include Mycophenolate, Bortezomib, Carfilzomib, Ixazomib, Daratumumab, Isatuximab and Elotuzumab. Non-limiting examples of an IL1 inhibitor include Anakinra, and Canakinumab. Non-limiting examples of a TNF inhibitor include Adalimumab, Certolizumab pegol, Etanercept, Golimumab, and Infliximab. Non-limiting examples of a Neutrophil function inhibitor include Dasatinib, Apremilast, and Roflumilast. Non-limiting examples of a NK cell inhibitor include Azathioprine. Non-limiting examples of a B cell inhibitor include Belimumab, Rituximab, Obinutuzumab, and Inebilizumab. In certain embodiments, the treatment for lupus comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Inebilizumab, or any combination thereof.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

The current disclosure includes the following aspects.
1. A method for classifying a lupus disease state of a patient, the method comprising:
   analyzing a data set comprising or derived from gene expression measurements of at least 2 genes selected from genes listed in Table 3, and Tables 5-1 to 5-20 to classify the lupus disease state of the patient,
wherein the gene expression measurements are obtained from a biological sample from the patient.
2. The method of aspect 1, wherein the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450 or 453 genes selected from genes listed in Table 3, and Tables 5-1 to 5-20.
3. The method of aspect 1, wherein the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 170, 180, 190, 200, 210, 220, 230, 240, 250, 260, 270, 280, 290, 300, 310, 320, 330, 340, 350, 360, 370, 380, 390, 400, or all genes selected from genes listed in Tables 5-1 to 5-4, 5-6 to 5-10, and 5-12 to 5-20.
4. The method of aspect 1, wherein the data set comprises or is derived from gene expression measurements of an effective number of genes selected from genes listed in each of one or more Tables selected from Tables 5-1 to 5-4, 5-6 to 5-10, and 5-12 to 5-20.
5. The method of aspect 1, wherein the data set comprises or is derived from gene expression measurements of an effective number of genes selected from genes listed in each of Tables selected from Tables 5-1 to 5-4, 5-6 to 5-10, and 5-12 to 5-20.
6. The method of any one of aspects 1 to 5, wherein the lupus disease state of the patient is classified with an accuracy of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
7. The method of any one of aspects 1 to 6, wherein the lupus disease state of the patient is classified with a sensitivity of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
8. The method of any one of aspects 1 to 7, wherein the lupus disease state of the patient is classified with a specificity of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
9. The method of any one of aspects 1 to 8, wherein the lupus disease state of the patient is classified with a positive predictive value of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
10. The method of any one of aspects 1 to 9, wherein the lupus disease state of the patient is classified with a negative predictive value of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
11. The method of any one of aspects 1 to 10, wherein the data set comprises an enrichment score derived from the gene expression measurements, and the enrichment score is analyzed to classify the lupus disease state of the patient.
12. The method of aspect 11, wherein the enrichment score is derived from the gene expression measurements using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof.
13. The method of aspect 11, wherein the enrichment score is derived from the gene expression measurements using GSVA.
14. The method of any one of aspects 1 to 13, wherein analyzing the data set comprises providing the data set as an input to a machine-learning model, wherein the machine learning model generates an inference indicative of the lupus disease state of the patient, based on the data set.
15. The method of aspect 14, wherein the method further comprises:
   receiving, as an output of the machine-learning model, the inference indicative of the lupus disease state of the patient; and
   electronically outputting a report classifying the lupus disease state of the patient.
16. The method of aspect 14 or 15, wherein the machine-learning model is trained using linear regression, logistic regression (LOG), Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), or any combination thereof.
17. The method of any one of aspects 14 to 16, wherein the inference includes a confidence value between 0 and 1.
18. The method of any one of aspects 14 to 17, wherein the machine learning model has a receiver operating characteristic (ROC) curve with an Area-Under-Curve (AUC) of at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.
19. The method of any one of aspects 1 to 13, wherein the analyzing the dataset comprises calculating a risk score for the patient based on the dataset, and classifying the lupus disease state of the patient based at least on the risk score.
20. The method of any one of aspects 1 to 19, wherein the biological sample comprises a blood sample, isolated peripheral blood mononuclear cells (PBMCs), or any derivative thereof.
21. The method of any one of aspects 1 to 20, wherein the data set comprises or is derived from gene expression measurements of at least 2 genes selected from the genes listed in Table 5-16, Table 5-15, Table 5-18, and Table 5-10.
22. The method of aspect 21, wherein the data set comprises or is derived from gene expression measurements of an effective number of genes selected from each of Table 5-16, Table 5-15, Table 5-18, and Table 5-10.
23. The method of aspect 21 or 22, wherein the method classifies whether the patient has lupus.
24. The method of any one of aspects 1 to 20, wherein the data set comprises or is derived from gene expression measurements of at least 2 genes selected from genes listed in Table 5-20, Table 5-19, Table 5-4, and Table 5-17.
25. The method of aspect 24, wherein the data set comprises or is derived from gene expression measurements of an effective number of genes selected from each of Table 5-20, Table 5-19, Table 5-4, and Table 5-17.
26. The method of aspect 24 or 25, wherein the method classifies whether the patient has active lupus, or inactive lupus.
27. The method of any one of aspects 1 to 26, further comprising administering a treatment to the patient based on the lupus disease state of the patient.
28. The method of aspect 27, wherein the treatment is configured to treat lupus.
29. The method of aspect 27, wherein the treatment is configured to reduce severity of lupus.
30. The method of aspect 27, wherein the treatment is configured to reduce a risk of developing lupus.
31. The method of any one of aspects 27 to 30, wherein the treatment comprises a pharmaceutical composition.
32. A method for diagnosing lupus in a patient, the method comprising detecting presence of one or more single nucleotide polymorphisms (SNPs) listed in Table 3, in a biological sample from the patient.
33. The method of aspect 32, wherein the method comprises detecting presence of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, or 135 SNPs, listed in Table 3.
34. The method of aspect 32 or 33, wherein the presence of the SNPs in the biological sample is detected by analyzing a nucleic acid of the patient in the biological sample.
35. The method of aspect 34, wherein analyzing the nucleic acid comprises sequencing at least a portion of DNA of the patient in the biological sample.
36. The method of aspect 34, wherein analyzing the nucleic acid comprises analyzing expression of the genes associated with the one or more SNPs.
37. The method of any one of aspects 32 to 36, wherein the biological sample comprises a blood sample, isolated peripheral blood mononuclear cells (PBMCs), or any derivative thereof.
38. The method of any one of aspects 32 to 37, wherein the method diagnoses lupus in the patient with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
39. The method of any one of aspects 32 to 38, wherein the method diagnoses lupus in the patient with a sensitivity of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
40. The method of any one of aspects 32 to 39, wherein the method diagnoses lupus in the patient with a specificity of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
41. The method of any one of aspects 32 to 40, wherein the method diagnoses lupus in the patient with a positive predictive value of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
42. The method of any one of aspects 32 to 41, wherein the method diagnoses lupus in the patient with a negative predictive value of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
43. The method of any one of aspects 32 to 42, further comprising administering a treatment to the patient based on the classified lupus disease state of the patient.
44. The method of aspect 43, wherein the treatment is configured to treat lupus.
45. The method of aspect 43, wherein the treatment is configured to reduce lupus severity.
46. The method of aspect 43, wherein the treatment is configured to reduce a risk of developing lupus.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-C: Biological characterization of the Primary Immunodeficiency Database. Breakdown of Primary Immunodeficiency Database contents by cell type/tissue of origin and biological function. FIG. 1A. Tissue and cell type enrichment shown as gene count for each I-SCOPE/T-SCOPE category. FIG. 1B. Biological functions represented within the database are displayed as percentage of total PID genes present in each BIG-C category. Native breakdown of all genes represented within the BIG-C tool is shown as percentage of total BIG-C genes present in each functional category. In each functional category the percent of PID genes is shown in the upper bar of each pair, and the percent of BIG-C genes is shown in the lower bar of each pair (e.g., in the category “unknown” the shorter upper bar shows PID genes, and the taller lower bar shows BIG-C genes). FIG. 1C. Interaction network of genes present within the PID gene database. Genes are colored according to mCODE cluster membership.

FIG. 2: GSVA enrichment analysis of BIG-C categories by PID gene mCODE cluster. Bubble Plots were generated using a custom R-script that simultaneously graphs enrichment odds ratios (circle size) and −log(p) values (circle shade). BIG-C categories (X axis) with larger circles and darker shades are the most enriched in the specified mCODE cluster (Y axis). “x” indicates no data.

FIGS. 3A-B: Monte Carlo analysis of overlap between SLE SNP-predicted PID genes and randomly selected protein-coding genes. Validation Monte Carlo analysis of the probability of producing the detected number of PID genes if using lists of randomly selected genes instead of SLE SNP-predicted genes. Simulations were performed using either all genes (FIG. 3A) or only protein-coding genes (FIG. 3B) as the potential pool for random gene selection as described in Methods.

FIGS. 4A-D: Protein-protein interaction network of SNP-predicted SLE risk genes. FIG. 4A. Interaction network of SNP-predicted SLE risk genes generated in Cytoscape and clustered via mCODE. Genes are annotated by type (filled circles without star, E-genes; white/empty circles, T-genes; diamonds, C-genes; filled circles with star, P-genes) and genes identified directly by SLE risk SNPs are labeled with SNP reference number. FIGS. 4B-C. Bubble plots showing cluster enrichment of BIG-C functional categories (FIG. 4B) and I-SCOPE cell category (FIG. 4C). Odds ratio is shown by bubble size and significance is shown by bubble color shading as −log(p). “x” indicates no data. FIG. 4D. Top pathways for each cluster by IPA canonical pathway analysis.

FIGS. 5A-D: PID genes are significantly differentially expressed in SLE patients. FIGS. 5A-B. Differential gene expression data from GSE49454 (FIG. 5A) and GSE45291 (FIG. 5B). Overexpressed genes are shown in lighter shade, underexpressed genes are shown in darker shade. Patient cohort (SLE or healthy control) is indicated at the bottom of each column. Results are shown following unsupervised hierarchical clustering. FIGS. 5C-D. Monte Carlo simulation results for random gene overlap with SLE patient DE (differentially expressed) genes. Simulations against random samples from the pool of all genes present on microarray were run 100,000 times each and resulting number of overlapping genes are shown as histograms. Lines indicate actual proportion of DE PID genes for each dataset.

FIGS. 6A-B: PID mCODE clusters show unique expression patterns among immune cell populations. FIG. 6A. Schematic of protein-protein interaction network of PID gene mCODE clusters. Node size correlates to number of genes in each cluster and node color maps to number of intracluster connections. Edge weight thickness represents number of intercluster connections and edge color is mapped to mCODE combined edge score. Each node is labeled with the most highly represented BIG-C category for its member genes. FIG. 6B. DE data from sorted cell datasets overlayed on PID mCODE network. Each node represents one gene, with overexpressed genes shown in squares with dark shade and underexpressed genes shown in squares with light shade. Genes that were not significantly DE are shown in grey circles. Datasets used for each panel include GSE39088 (whole blood), GSE50772 (PBMC), GSE4588 (CD19 B cells), and GSE51997 (CD4 T cells, classical CD14⁺CD16⁻ monocytes, and nonclassical CD14⁺CD16⁺ monocytes).

FIG. 7: GSVA enrichment of PID mCODE clusters within GSE88884 SLE patient dataset. Heatmap of GSVA enrichment of PID mCODE cluster gene lists within each patient in GSE88884, sorted by unsupervised hierarchical clustering. Column breaks in the heatmap are placed between the three largest groups produced by the hierarchical clustering dendrogram.

FIGS. 8A-E: mCODE-derived PID gene clusters can identify clinically meaningful patient groups. FIG. 8A. GSVA of SLE patient DE gene data (GSE88884) using PID mCODE clusters as input gene sets. Output is shown following directed hierarchical clustering set to k=3 (clustering groups are shown as colored and numbered bars between heatmap and dendrogram). FIG. 8B. Clinical data summary and statistics of the three groups resulting from directed hierarchical clustering. *, p<0.05; **, p<0.001; ***, p<0.0001. FIG. 8C. Total PID gene DE profile of patients within GSE88884, shown as log FC analysis for all patients combined, inactive (SLEDAI<6) patients only, or active (SLEDAI≥6) patients only. FIG. 8D. Variational autoencoder results displayed as DE values (row z-score) for each of the 5 autoencoder-derived groups, separated into Illuminate-1 and Illuminate-2 arms of trial data. FIG. 8E. Variational autoencoder results displayed as GSVA enrichment of PID mCODE clusters (row z-score) for each of the 5 autoencoder-derived groups per trial arm. −ve row z-score are denoted by white asterisk (*).

FIGS. 9A-C: PID gene clusters show utility as ML classifiers for SLE patient disease state. FIGS. 9A-B. ROC curves for 9 ML classifiers trained using PID mCODE clusters to correctly sort SLE patients from healthy controls (FIG. 9A) or active SLE patients from inactive SLE patients (FIG. 9B). FIG. 9C. Top feature clusters for ML identification of SLE vs control (left) or active SLE vs inactive SLE (right) across all classifiers. Overall feature importance data is mapped onto the PID mCODE schematic by node color, and clusters with positive feature importance values are annotated by defining BIG-C functional category.

FIG. 10: Individual machine learning classifier performance comparison. Receiver operator characteristic curves are shown separately for each of the nine machine learning classifiers tested in FIGS. 9A-C. Each classifier was run over a 6-fold testing protocol (individual folds shown as thin colored lines) and a mean ROC curve (thick blue line) was calculated for each to assess average expected performance. The confidence interval±1 standard deviation for each 6-fold validation is shown in grey for each panel. For ADB receiver operating characteristic curve ROC fold 0 AUC is 0.85, ROC fold 1 AUC is 0.84, ROC fold 2 AUC is 0.76, ROC fold 3 AUC is 0.84, ROC fold 4 AUC is 0.69, ROC fold 5 AUC is 0.69, ROC fold 6 AUC is 0.73, and Mean ROC AUC is 0.77±0.07. For DTREE receiver operating characteristic curve ROC fold 0 AUC is 0.66, ROC fold 1 AUC is 0.76, ROC fold 2 AUC is 0.69, ROC fold 3 AUC is 0.72, ROC fold 4 AUC is 0.60, ROC fold 5 AUC is 0.56, ROC fold 6 AUC is 0.75, and Mean ROC AUC is 0.68±0.07. For GB receiver operating characteristic curve ROC fold 0 AUC is 0.84, ROC fold 1 AUC is 0.89, ROC fold 2 AUC is 0.76, ROC fold 3 AUC is 0.86, ROC fold 4 AUC is 0.78, ROC fold 5 AUC is 0.71, ROC fold 6 AUC is 0.82, and Mean ROC AUC is 0.81±0.06. For KNN receiver operating characteristic curve ROC fold 0 AUC is 0.78, ROC fold 1 AUC is 0.89, ROC fold 2 AUC is 0.78, ROC fold 3 AUC is 0.87, ROC fold 4 AUC is 0.84, ROC fold 5 AUC is 0.75, ROC fold 6 AUC is 0.87, and Mean ROC AUC is 0.83±0.05. For LDA receiver operating characteristic curve ROC fold 0 AUC is 0.50, ROC fold 1 AUC is 0.55, ROC fold 2 AUC is 0.50, ROC fold 3 AUC is 0.54, ROC fold 4 AUC is 0.50, ROC fold 5 AUC is 0.59, ROC fold 6 AUC is 0.54, and Mean ROC AUC is 0.53±0.03. For NB receiver operating characteristic curve ROC fold 0 AUC is 0.78, ROC fold 1 AUC is 0.78, ROC fold 2 AUC is 0.74, ROC fold 3 AUC is 0.83, ROC fold 4 AUC is 0.76, ROC fold 5 AUC is 0.75, ROC fold 6 AUC is 0.74, and Mean ROC AUC is 0.77±0.03. For RF receiver operating characteristic curve ROC fold 0 AUC is 0.85, ROC fold 1 AUC is 0.85, ROC fold 2 AUC is 0.76, ROC fold 3 AUC is 0.89, ROC fold 4 AUC is 0.77, ROC fold 5 AUC is 0.75, ROC fold 6 AUC is 0.84, and Mean ROC AUC is 0.81±0.05. For SVM receiver operating characteristic curve ROC fold 0 AUC is 0.83, ROC fold 1 AUC is 0.91, ROC fold 2 AUC is 0.80, ROC fold 3 AUC is 0.90, ROC fold 4 AUC is 0.84, ROC fold 5 AUC is 0.80, ROC fold 6 AUC is 0.90, and Mean ROC AUC is 0.85±0.04. For LR receiver operating characteristic curve ROC fold 0 AUC is 0.50, ROC fold 1 AUC is 0.54, ROC fold 2 AUC is 0.50, ROC fold 3 AUC is 0.54, ROC fold 4 AUC is 0.49, ROC fold 5 AUC is 0.61, ROC fold 6 AUC is 0.51, and Mean ROC AUC is 0.53±0.04.

### DETAILED DESCRIPTION

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the term “Gini impurity” refers to a measure of how often a randomly chosen element from the set may be incorrectly labeled if it is randomly labeled according to the distribution of labels in the subset.

Many complex and multi-systematic diseases and conditions currently pose major diagnostic and therapeutic challenges. Despite the wealth of records from, for example, genetic, epigenetic, and gene expression data that has emerged in the past few years, physicians often still rely on clinical evaluation and laboratory tests, including measurement of autoantibodies and complement levels.

Successful relation of records (e.g., gene expression records) to a specific disease phenotype activity has been attempted, including efforts to identify individual genes that predicted subsequent flares, and through the determination of a discrete group of differentially expressed (DE) genes that may be found in a particular record. Despite these advances, however, no such approach is available with sufficient predictive value to utilize in evaluation and treatment.

As such, there is a need for a predictive tool for evaluating patient at both the chemical and cellular levels to advance personalized treatment. Data analytical techniques such as machine learning enable proper correlation between genetic records and phenotypes.

The machine learning models tested here provide the basis of personalized medicine. Integration of the methods herein with emerging high-throughput record sampling technologies may unlock the potential to develop a simple blood test to predict phenotypic activity. The disclosures herein may be generalized to predict other manifestations, such as organ involvement. A better understanding of the cellular processes that drive pathogenesis may eventually lead to customized therapeutic strategies based on records' unique patterns of cellular activation.

One aspect of the present disclosure is directed to a method for diagnosing lupus in a patient. The method comprises detecting presence of one or more single nucleotide polymorphisms (SNPs) selected from the SNPs listed in Table 3, in a biological sample from the patient. Detecting presence of the one or more SNPs, in a biological sample can include detecting whether or not the one or more SNPs are present in the biological sample. The patient is determined to have lupus, or is at risk of developing lupus when the one or more SNPs are present in the biological sample. Lupus can be any type of lupus including but not limited to systemic lupus erythematosus (SLE), cutaneous lupus erythematosus, drug-induced lupus, and neonatal lupus. In certain embodiments, the lupus is SLE.

In certain embodiments, the one or more SNPs comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, or 135 SNPs.

In certain embodiments, the one or more SNPs comprises 2 SNPs to 135 SNPs, e.g., the method includes detecting presence of the 2 to 135 SNPs selected from the SNPs listed in Table 3, in the biological sample from the patient, and the patient is determined to have lupus, or is at risk of developing lupus when the one or more SNPs are present in the biological sample. In certain embodiments, the one or more SNPs comprises 2 SNPs to 5 SNPs, 2 SNPs to 10 SNPs, 2 SNPs to 20 SNPs, 2 SNPs to 30 SNPs, 2 SNPs to 40 SNPs, 2 SNPs to 50 SNPs, 2 SNPs to 70 SNPs, 2 SNPs to 90 SNPs, 2 SNPs to 100 SNPs, 2 SNPs to 120 SNPs, 2 SNPs to 135 SNPs, 5 SNPs to 10 SNPs, 5 SNPs to 20 SNPs, 5 SNPs to 30 SNPs, 5 SNPs to 40 SNPs, 5 SNPs to 50 SNPs, 5 SNPs to 70 SNPs, 5 SNPs to 90 SNPs, 5 SNPs to 100 SNPs, 5 SNPs to 120 SNPs, 5 SNPs to 135 SNPs, 10 SNPs to 20 SNPs, 10 SNPs to 30 SNPs, 10 SNPs to 40 SNPs, 10 SNPs to 50 SNPs, 10 SNPs to 70 SNPs, 10 SNPs to 90 SNPs, 10 SNPs to 100 SNPs, 10 SNPs to 120 SNPs, 10 SNPs to 135 SNPs, 20 SNPs to 30 SNPs, 20 SNPs to 40 SNPs, 20 SNPs to 50 SNPs, 20 SNPs to 70 SNPs, 20 SNPs to 90 SNPs, 20 SNPs to 100 SNPs, 20 SNPs to 120 SNPs, 20 SNPs to 135 SNPs, 30 SNPs to 40 SNPs, 30 SNPs to 50 SNPs, 30 SNPs to 70 SNPs, 30 SNPs to 90 SNPs, 30 SNPs to 100 SNPs, 30 SNPs to 120 SNPs, 30 SNPs to 135 SNPs, 40 SNPs to 50 SNPs, 40 SNPs to 70 SNPs, 40 SNPs to 90 SNPs, 40 SNPs to 100 SNPs, 40 SNPs to 120 SNPs, 40 SNPs to 135 SNPs, 50 SNPs to 70 SNPs, 50 SNPs to 90 SNPs, 50 SNPs to 100 SNPs, 50 SNPs to 120 SNPs, 50 SNPs to 135 SNPs, 70 SNPs to 90 SNPs, 70 SNPs to 100 SNPs, 70 SNPs to 120 SNPs, 70 SNPs to 135 SNPs, 90 SNPs to 100 SNPs, 90 SNPs to 120 SNPs, 90 SNPs to 135 SNPs, 100 SNPs to 120 SNPs, 100 SNPs to 135 SNPs, or 120 SNPs to 135 SNPs. In certain embodiments, the one or more SNPs comprises 2 SNPs, 5 SNPs, 10 SNPs, 20 SNPs, 30 SNPs, 40 SNPs, 50 SNPs, 70 SNPs, 90 SNPs, 100 SNPs, 120 SNPs, or 135 SNPs. In certain embodiments, the one or more SNPs comprises at least 2 SNPs, 5 SNPs, 10 SNPs, 20 SNPs, 30 SNPs, 40 SNPs, 50 SNPs, 70 SNPs, 90 SNPs, 100 SNPs, or 120 SNPs.

The presence of the one or more SNPs in the biological sample can be detected by analyzing nucleic acid of the patient in the biological sample. The nucleic acid can be DNA, and/or RNA. In certain embodiments, analyzing the nucleic acid of the patient in the biological sample can include sequencing at least a portion of the DNA of the patient in the biological sample. The at least a portion of the DNA can include expected chromosomal location of the one or more SNPs. In certain embodiments, analyzing the nucleic acid of the patient in the biological sample can include sequencing the DNA of the patient in the biological sample. The DNA can be sequenced using any suitable method including but not limited to Sanger sequencing, next-generation sequencing, capillary electrophoresis, fragment analysis, or any combination thereof. In certain embodiments, analyzing the nucleic acid of the patient in the biological sample can include sequencing and quantification of at least a portion of the RNA of the patient in the biological sample. In certain embodiments, analyzing the nucleic acid of the patient in the biological sample can include sequencing and quantification of the RNA of the patient in the biological sample. RNA can be any RNA as desired to be analyzed by one of skill in the art e.g., total RNA, mRNA, poly A RNA, non-coding RNA, etc. In certain embodiments, analyzing the nucleic acid of the patient comprises analyzing expression of the genes associated with the one or more SNPs. RNA sequencing and quantification, and/or gene expression analysis can be performed using any suitable method including but not limited to RNA sequencing, microarray analysis, RNA-Seq, qPCR, northern blotting, fluorescent in situ hybridization, serial analysis of gene expression, tiling arrays or any combination thereof. A gene associated with a SNP can include a gene, expression of which in a biological sample may depend on presence or absence of the SNP in the biological sample. In Table 3, for a respective SNP, the associated genes are listed in the same row.

The biological sample can be obtained or derived from the patient. The biological sample can contain a blood sample, isolated peripheral blood mononuclear cells (PBMCs), tissue biopsy sample, nasal fluid, saliva, urine, stool, or any derivative thereof. In certain embodiments, the biological sample contains a blood sample or any derivative thereof. In certain embodiments, the biological sample contains PBMCs or any derivative thereof. In certain embodiments, the biological sample contains a tissue biopsy sample or any derivative thereof. In certain embodiments, the biological sample contains a nasal fluid sample or any derivative thereof. In certain embodiments, the biological sample contains a saliva sample or any derivative thereof. In certain embodiments, the biological sample contains a urine sample or any derivative thereof. In certain embodiments, the biological sample contains a stool sample or any derivative thereof.

In certain embodiments, the method can determine whether or not the patient has lupus, or is at risk of developing lupus with an accuracy of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In certain embodiments, the method can determine whether or not the patient has lupus, or is at risk of developing lupus with a sensitivity of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In certain embodiments, the method can determine whether or not the patient has lupus, or is at risk of developing lupus with a specificity of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In certain embodiments, the method can determine whether or not the patient has lupus, or is at risk of developing lupus with a positive predictive value of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In certain embodiments, the method can determine whether or not the patient has lupus, or is at risk of developing lupus with a negative predictive value of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In certain embodiments, the method further comprises administering a treatment to the patient. The treatment can be administered based on the determination that the patient has lupus, or is at risk of developing lupus. In certain embodiments, the treatment is configured to treat lupus. In certain embodiments, the treatment is configured to reduce severity of lupus. In certain embodiments, the treatment is configured to reduce a risk of having lupus. In certain embodiments, the treatment is configured to treat active lupus. In certain embodiments, the treatment is configured to reduce severity of active lupus. In certain embodiments, the treatment is configured to reduce a risk of having active lupus. In certain embodiments, the treatment is configured to treat inactive lupus. In certain embodiments, the treatment is configured to reduce severity of inactive lupus. In certain embodiments, the treatment is configured to reduce a risk of having inactive lupus.

In certain embodiments, the treatment for lupus comprises a neutrophil function inhibitor, a TNF inhibitor, an IL1 inhibitor, a Plasma cell inhibitor, a NK cell inhibitor, a B Cell Inhibitor, or any combination thereof. Non-limiting examples of an IFN inhibitor include Anifrolumab. Non-limiting examples of a Plasma cell inhibitor include Mycophenolate, Bortezomib, Carfilzomib, Ixazomib, Daratumumab, Isatuximab and Elotuzumab. Non-limiting examples of an IL1 inhibitor include Anakinra, and Canakinumab. Non-limiting examples of a TNF inhibitor include Adalimumab, Certolizumab pegol, Etanercept, Golimumab, and Infliximab. Non-limiting examples of a Neutrophil function inhibitor include Dasatinib, Apremilast, and Roflumilast. Non-limiting examples of a NK cell inhibitor include Azathioprine. Non-limiting examples of a B cell inhibitor include Belimumab, Rituximab, Obinutuzumab, and Inebilizumab. In certain embodiments, the treatment for lupus comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Inebilizumab, or any combination thereof.

The patient can be a human patient. In certain embodiments, the patient has lupus. In certain embodiments, the patient is asymptomatic of lupus. In certain embodiments, the patient is suspected of having lupus. In certain embodiments, the patient has active lupus. In certain embodiments, the patient is suspected of having active lupus. In certain embodiments, the patient has inactive lupus. In certain embodiments, the patient is suspected of having inactive lupus.

An aspect of the present disclosure is directed to a method for classifying the lupus disease state of a patient. The method can include analyzing a data set comprising or derived from gene expression measurements of at least 2 genes to classify the lupus disease state of the patient. The gene expression measurements can be obtained from a biological sample obtained or derived from the patient. In certain embodiments, classifying the lupus disease state of the patient includes classifying (e.g., determining) whether the patient has lupus. In certain embodiments, classifying the lupus disease state of the patient includes classifying (e.g., determining) whether or not the patient has lupus, and the data set is analyzed to classify whether or not the patient has lupus. In certain embodiments, classifying the lupus disease state of the patient includes classifying (e.g., determining) whether the patient has active lupus or inactive lupus. In certain embodiments, classifying the lupus disease state of the patient includes classifying (e.g., determining) whether the patient has active lupus or inactive lupus, and the data set is analyzed to classify whether the patient has active lupus or inactive lupus. In certain embodiments, classifying the lupus disease state of the patient includes classifying (e.g., determining) whether the patient has active lupus, inactive lupus, or does not have lupus. In certain embodiments, classifying the lupus disease state of the patient includes classifying (e.g., determining) whether the patient has active lupus, inactive lupus, or does not have lupus, and the data set is analyzed to classify whether the patient has active lupus, inactive lupus, or does not have lupus.

Lupus can be any type of lupus including but not limited to systemic lupus erythematosus (SLE), cutaneous lupus erythematosus, drug-induced lupus, and neonatal lupus. In certain embodiments, the lupus is SLE.

In certain embodiments, the at least 2 genes of the data set are selected from genes listed in Table 3, and Tables 5-1 to 5-20, i.e., the data set comprises or is derived from gene expression measurements of at least 2 genes selected from the genes listed in Table 3, and Tables 5-1 to 5-20, from the biological sample from the patient. In certain embodiments, the at least 2 genes of the data set are selected from the genes listed in Table 3. In certain embodiments, the at least 2 genes of the data set are selected from genes listed in Tables 5-1 to 5-20. In certain embodiments, the at least 2 genes of the data set are selected from genes listed in Tables 5-1 to 5-4, 5-6 to 5-10, and 5-12 to 5-20. Genes listed in Tables 5-1, to 5-20, include all the genes, e.g., the 453 genes listed in Tables 5-1 to 5-20. As a non-limiting example, “genes listed in Table X and Y” includes x+y genes, where Table X contains x genes and Table Y contains y genes, considering no overlap (e.g., the genes are different) exists between x and y genes. In the event of overlap, duplicate copies can be excluded from analysis. The at least 2 genes may or may not include any gene that is not listed in Table 3, and Tables 5-1 to 5-20. In certain embodiments, the at least 2 genes do not include any gene that is not listed in Table 3, and Tables 5-1 to 5-20. In certain embodiments, the at least 2 genes do not include any gene that is not listed in Tables 5-1 to 5-20. In certain embodiments, the at least 2 genes do not include any gene that is not listed in Tables 5-1 to 5-4, 5-6 to 5-10, and 5-12 to 5-20.

In certain embodiments, the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, or 453 genes, selected from genes listed in Table 3, and Tables 5-1 to 5-20, from the biological sample from the patient. In certain embodiments, the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, or all genes, selected from genes listed in Table 3, from the biological sample from the patient. In certain embodiments, the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, or 453 genes, selected from genes listed in Tables 5-1 to 5-20, from the biological sample from the patient. In certain embodiments, the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 445, or all genes, selected from genes listed in Tables 5-1 to 5-4, 5-6 to 5-10, and 5-12 to 5-20, from the biological sample from the patient. In certain embodiments, the data set comprises or is derived from gene expression measurements of the genes listed in Tables 5-1 to 5-4, 5-6 to 5-10, and 5-12 to 5-20, from the biological sample from the patient.

In certain embodiments, the data set comprises or is derived from gene expression measurements of 1 to all genes, selected from genes listed in Tables 5-1 to 5-4, 5-6 to 5-10, and 5-12 to 5-20, from the biological sample from the patient. In certain embodiments, the data set comprises or is derived from gene expression measurements of 1 to 5, 1 to 10, 1 to 50, 1 to 100, 1 to 150, 1 to 200, 1 to 250, 1 to 300, 1 to 400, 1 to 445, 1 to all, 5 to 10, 5 to 50, 5 to 100, 5 to 150, 5 to 200, 5 to 250, 5 to 300, 5 to 400, 5 to 445, 5 to all, 10 to 50, 10 to 100, 10 to 150, 10 to 200, 10 to 250, 10 to 300, 10 to 400, 10 to 445, 10 to all, 50 to 100, 50 to 150, 50 to 200, 50 to 250, 50 to 300, 50 to 400, 50 to 445, 50 to all, 100 to 150, 100 to 200, 100 to 250, 100 to 300, 100 to 400, 100 to 445, 100 to all, 150 to 200, 150 to 250, 150 to 300, 150 to 400, 150 to 445, 150 to all, 200 to 250, 200 to 300, 200 to 400, 200 to 445, 200 to all, 250 to 300, 250 to 400, 250 to 445, 250 to all, 300 to 400, 300 to 445, 300 to all, 400 to 445, 400 to all, or 445 to all, genes selected from genes listed in Tables 5-1 to 5-4, 5-6 to 5-10, and 5-12 to 5-20, from the biological sample from the patient. In certain embodiments, the data set comprises or is derived from gene expression measurements of 1, 5, 10, 50, 100, 150, 200, 250, 300, 400, 445, or all, genes selected from genes listed in Tables 5-1 to 5-4, 5-6 to 5-10, and 5-12 to 5-20, from the biological sample from the patient. In certain embodiments, the data set comprises or is derived from gene expression measurements of at least 1, 5, 10, 50, 100, 150, 200, 250, 300, 400, or 445, genes selected from genes listed in Tables 5-1 to 5-4, 5-6 to 5-10, and 5-12 to 5-20, from the biological sample from the patient.

In certain embodiments, the data set comprises or is derived from gene expression measurements of at least 2 genes selected from the genes listed in each of one or more Tables selected from Tables 5-1 to 5-20, from the biological sample from the patient. In a non-limiting example, Table 5-16, Table 5-15, Table 5-18, and Table 5-10, are selected, i.e., the data set comprises or is derived from gene expression measurements of at least 2 genes selected from genes listed in each of the selected tables (Table 5-16, Table 5-15, Table 5-18, and Table 5-10), i.e., the data set comprises or is derived from gene expression measurements of at least 2 genes selected from the genes listed in Table 5-16, at least 2 genes selected from the genes listed in Table 5-15, at least 2 genes selected from the genes listed in Table 5-18, and at least 2 genes selected from the genes listed in Table 5-10. In certain embodiments, the data set comprises or is derived from gene expression measurements of at least 2 genes selected from the genes listed in each of one or more Tables selected from Tables 5-1 to 5-4, 5-6 to 5-10, and 5-12 to 5-20, from the biological sample from the patient. In certain embodiments, the data set comprises or is derived from gene expression measurements of at least 2 genes selected from the genes listed in each of 2, 3, 4, 5, 6, 7, 8, 9, 10, 11, 12, 13, 14, 15, 16, 17, 18, 19, 20, or 2 to 20, or 5 to 20, or 10 to 20, or 15 to 20 or any range there between, Tables selected from Tables 5-1 to 5-20, from the biological sample from the patient. In certain embodiments, the data set comprises or is derived from gene expression measurements of at least 2 genes selected from the genes listed in each of 2, 3, 4, 5, 6, 7, 8, 9, 10, 11, 12, 13, 14, 15, 16, 17, or 18, or 2 to 18, or 5 to 18, or 10 to 18, or 15 to 18 or any range there between, Tables selected from Tables 5-1 to 5-4, 5-6 to 5-10, and 5-12 to 5-20, from the biological sample from the patient. In certain embodiments, Tables 5-1 to 5-4, 5-6 to 5-10, and 5-12 to 5-20 are selected. In certain embodiments, the data set comprises or is derived from gene expression measurements of at least 2 genes selected from the genes listed in each Tables selected from Tables 5-1 to 5-4, 5-6 to 5-10, and 5-12 to 5-20, from the biological sample from the patient, i.e, the data set comprises or is derived from gene expression measurements of, at least 2 genes selected from the genes listed in Table 5-1; at least 2 genes selected from the genes listed in Table 5-2; at least 2 genes selected from the genes listed in Table 5-3; at least 2 genes selected from the genes listed in Table 5-4; at least 2 genes selected from the genes listed in Table 5-6; at least 2 genes selected from the genes listed in Table 5-7; at least 2 genes selected from the genes listed in Table 5-8; at least 2 genes selected from the genes listed in Table 5-9; at least 2 genes selected from the genes listed in Table 5-10; at least 2 genes selected from the genes listed in Table 5-12; at least 2 genes selected from the genes listed in Table 5-13; at least 2 genes selected from the genes listed in Table 5-14; at least 2 genes selected from the genes listed in Table 5-15; at least 2 genes selected from the genes listed in Table 5-16; at least 2 genes selected from the genes listed in Table 5-17; at least 2 genes selected from the genes listed in Table 5-18; at least 2 genes selected from the genes listed in Table 5-19; and at least 2 genes selected from the genes listed in Table 5-20, from the biological sample from the patient, wherein the number of genes selected from different selected Tables can be the same or different.

In certain embodiments, for each selected Table, the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, or 135, or all, or any range there between, genes selected from the genes listed in the selected Table, from the biological sample from the patient, wherein the number of genes selected from different selected Tables can be the same or different. In certain embodiments, for each selected Table, the data set comprises or is derived from gene expression measurements of an effective number of genes selected from the genes listed in the selected Table, from the biological sample from the patient, wherein the number of genes selected from different selected Tables, and/or the effective number of genes selected from different selected Tables can be the same or different. In certain embodiments, the data set comprises or is derived from gene expression measurements of an effective number of genes selected from the genes listed in each of 2, 3, 4, 5, 6, 7, 8, 9, 10, 11, 12, 13, 14, 15, 16, 17, or 18, or 2 to 18, or 5 to 18, or 10 to 18, or 15 to 18 or any range there between, Tables selected from Tables 5-1 to 5-4, 5-6 to 5-10, and 5-12 to 5-20, from the biological sample from the patient, wherein the number of genes selected from different selected Tables can be same or different. In certain embodiments, the data set comprises or is derived from gene expression measurements of an effective number of genes selected from the genes listed in each Tables selected from Tables 5-1 to 5-4, 5-6 to 5-10, and 5-12 to 5-20, from the biological sample from the patient, i.e., the data set comprises or is derived from gene expression measurements of, an effective number of genes selected from the genes listed in Table 5-1; an effective number of genes selected from the genes listed in Table 5-2; an effective number of genes selected from the genes listed in Table 5-3; an effective number of genes selected from the genes listed in Table 5-4; an effective number of genes selected from the genes listed in Table 5-6; an effective number of genes selected from the genes listed in Table 5-7; an effective number of genes selected from the genes listed in Table 5-8; an effective number of genes selected from the genes listed in Table 5-9; an effective number of genes selected from the genes listed in Table 5-10; an effective number of genes selected from the genes listed in Table 5-12; an effective number of genes selected from the genes listed in Table 5-13; an effective number of genes selected from the genes listed in Table 5-14 an effective number of genes selected from the genes listed in Table 5-15; an effective number of genes selected from the genes listed in Table 5-16; an effective number of genes selected from the genes listed in Table 5-17; an effective number of genes selected from the genes listed in Table 5-18; an effective number of genes selected from the genes listed in Table 5-19; and an effective number of genes selected from the genes listed in Table 5-20, from the biological sample from the patient, wherein the number of genes selected from different selected Tables can be the same or different. In certain embodiments, the data set comprises or is derived from gene expression measurements of the genes listed in each of 2, 3, 4, 5, 6, 7, 8, 9, 10, 11, 12, 13, 14, 15, 16, 17, or 18, or 2 to 18, or 5 to 18, or 10 to 18, or 15 to 18 or any range there between, Tables selected from Tables 5-1 to 5-4, 5-6 to 5-10, and 5-12 to 5-20, from the biological sample from the patient. The selected genes of the data set, i.e., expression measurements of which the dataset is comprised of or derived from, may or may not include any gene that is not listed within the genes listed in Table 3, and Tables 5-1 to 5-20. In certain embodiments, the selected genes of the data set do not include any gene that is not listed within the genes listed in Tables 5-1 to 5-20. In certain embodiments, the selected genes of the data set do not include any gene that is not listed within the genes listed in Tables 5-1 to 5-4, 5-6 to 5-10, and 5-12 to 5-20. Selecting effective number of genes from a Table can include selecting at least minimum number of genes from the table to obtain desired accuracy, sensitivity, specificity, positive predictive value, and/or negative predictive value in classification of the lupus disease state of the patient. Desired accuracy, sensitivity, specificity, positive predictive value, and/or negative predictive value, can be an accuracy, sensitivity, specificity, positive predictive value, and/or negative predictive value described herein. In certain embodiments, effective number of genes for a module/Table can be determined using adjusted rand index (ARI) method. The ARI method can include performing k-Means clustering on randomly selected gene subsets by standard interval based on the total number of genes of each module/Table. Similarity between two clustering can be measured by adjusted rand index (ARI). As a non-limiting example, the adjusted rand index (ARI) can be calculated between k-Means cluster memberships from the randomly selected gene subsets to the cluster memberships obtained using total number of genes of each module/Table. The higher the ARI, the similar the cluster memberships and lower the ARI the weaker the cluster memberships, suggesting more genes may be required. The ARI can be calculated to determine the appropriate number of genes for each module. In certain embodiments, selecting effective number of genes from a Table (e.g., one of Tables 5-1 to 5-20) can include selecting at least 60%, 70%, 80%, 90%, or all genes from the Table. In certain embodiments, selecting effective number of genes from a Table (e.g., one of Tables 5-1 to 5-20) can include selecting at least 60% of the genes from the Table In certain embodiments, selecting effective number of genes from a Table (e.g., one of Tables 5-1 to 5-20) can include selecting at least 70% of the genes from the Table. In certain embodiments, selecting effective number of genes from a Table (e.g., one of Tables 5-1 to 5-20) can include selecting all genes from the Table.

The data set can be generated from the biological sample obtained or derived from the patient. For example, nucleic acid molecules of the patient in the biological sample can be assessed to obtain the data set. In certain embodiments, the gene expression measurements of the at least 2 genes (e.g., gene expression measurements of which the dataset is comprised of or derived from) in the biological sample can be performed using any suitable method known to those of skill in the art including but not limited to DNA sequencing, RNA sequencing, microarray, RNA-Seq, qPCR, northern blotting, fluorescent in situ hybridization, serial analysis of gene expression, tiling arrays or any combination thereof, to obtain the data set. In certain embodiments, the gene expression measurements can be performed using RNA-Seq. In certain embodiments, the gene expression measurements can be performed using microarray analysis. In certain embodiments, the gene expression measurements of the at least 2 genes in the biological sample can be performed using RNA-Seq, to obtain the data set. In certain embodiments, the gene expression measurements of the at least 2 genes in the biological sample can be performed using microarray analysis, to obtain the data set. In certain embodiments, the data set is derived from the gene expression measurements from the biological sample, wherein the gene expression measurements is analyzed using a suitable data analysis tool including but not limited to BIG-C™ big data analysis tool, an I-Scope™ big data analysis tool, a T-Scope™ big data analysis tool, a CellScan big data analysis tool, an MS (Molecular Signature) Scoring™ analysis tool, gene set variation analysis (GSVA), gene set enrichment analysis (GSEA), enrichment algorithm, Z score, multiscale embedded gene co-expression network analysis (MEGENA), weighted gene co-expression network analysis (WGCNA), differential expression analysis, log 2 expression analysis, or any combination thereof, to obtain the dataset. In certain embodiments, the gene expression measurements is analyzed using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof, to obtain the data set. In certain embodiments, the data set is derived from the gene expression measurements using GSVA. In certain embodiments, the method includes performing gene expression measurements of the at least 2 genes from the biological sample to obtain the dataset. In certain embodiments, the method includes analyzing the gene expression measurements of the at least 2 genes using a suitable data analysis tool to obtain the dataset. In certain embodiments, the method includes performing gene expression measurements of the at least 2 genes, and analyzing the gene expression measurements of the at least 2 genes using a suitable data analysis tool to obtain the dataset.

In certain embodiments, the data set is derived from the gene expression measurements (e.g., of the at least 2 genes) using GSVA. In certain embodiments, the data set is derived from the gene expression measurements using GSVA, and the data set comprises one or more GSVA scores of the patient, and the one or more GSVA scores of the patient is analyzed to classify the lupus disease state of the patient. The one or more GSVA scores can form an enrichment score of the patient. The one or more GSVA scores of the patient can be generated based on one or more Tables selected from Tables 5-1 to 5-20, wherein for each selected Table, at least one GSVA score of the patient is generated based on enrichment of expression of at least 2 genes selected from the genes listed in the selected Table, in the biological sample. In certain embodiments, the one or more GSVA scores of the patient can be generated based on one or more Tables selected from Tables 5-1 to 5-4, 5-6 to 5-10, and 5-12 to 5-20, wherein for each selected Table, at least one GSVA score of the patient is generated based on enrichment of expression of at least 2 genes selected from the genes listed in the selected Table, in the biological sample. In certain embodiments, for each selected Table, the at least one GSVA score of the patient based on the selected Table is generated based on enrichment of expression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 203, or all, any range or value there between genes selected from the genes listed in the respective Table, in the biological sample, wherein the number of gene selected from different selected Tables may be the same or different. In certain embodiments, for each selected Table, the at least one GSVA score of the patient based on the selected Table is generated based on enrichment of expression of an effective number of genes selected from the genes listed in the respective Table, in the biological sample, wherein the number of genes selected from different selected Tables may be the same or different. In certain embodiments, for each selected Table, the at least one GSVA score of the patient based on the selected Table is generated based on enrichment of expression of the genes listed in the respective Table, in the biological sample. The one or more GSVA scores can contain the at least one GSVA score generated from each of the selected Table, as a non-limiting example 4 Tables, such as Table 5-16, Table 5-15, Table 5-18, and Table 5-10 are selected, the one or more GSVA scores comprise, at least 1 score based on each selected Tables, i.e., at least 4 GSVA scores, at least 1 GSVA score generated based on Table 5-16, at least 1 GSVA score generated based on Table 5-15, at least 1 GSVA score generated based on Table 5-18, and at least 1 GSVA score generated based on Table 5-10, wherein the at least 1 GSVA score based on Table 5-16 is generated based on enrichment of expression of the genes selected (e.g. the at least 2 genes, effective number of genes, or all genes) from Table 5-16, in the biological sample; the at least 1 GSVA score based on Table 5-15 is generated based on enrichment of expression of the genes selected from Table 5-15, in the biological sample; the at least 1 GSVA score based on Table 5-18 is generated based on enrichment of expression of the genes selected from Table 5-18, in the biological sample; and the at least 1 GSVA score based on Table 5-10 is generated based on enrichment of expression of the genes selected from Table 5-10, in the biological sample. The gene selected (e.g. the at least 2 genes, effective number of genes, or all genes) from a respective selected Table, can form the input gene set for generating the at least one GSVA score of the patient based on the respective selected Table, using GSVA. In certain embodiments, one GSVA score is generated based on each of the selected Table, as a non-limiting example 4 Tables, such as Table 5-16, Table 5-15, Table 5-18, and Table 5-10 are selected, the one or more GSVA scores comprise, 1 score based on each selected Tables, i.e., 4 GSVA scores, 1 GSVA score generated based on Table 5-16, 1 GSVA score generated based on Table 5-15, 1 GSVA score generated based on Table 5-18, and 1 GSVA score generated based on Table 5-10, wherein the 1 GSVA score based on Table 5-16 is generated based on enrichment of expression of the genes selected from Table 5-16, in the biological sample; the 1 GSVA score based on Table 5-15 is generated based on enrichment of expression of the genes selected from Table 5-15, in the biological sample; the 1 GSVA score based on Table 5-18 is generated based on enrichment of expression of the genes selected from Table 5-18, in the biological sample; and the 1 GSVA score based on Table 5-10 is generated based on enrichment of expression of the genes selected from Table 5-10, in the biological sample. In certain embodiments, 1, 2, 3, 4, 5, 6, 7, 8, 9, 10, 11, 12, 13, 14, 15, 16, 17, 18, 19 or 20, or any range there between tables are selected from Tables 5-1 to 5-20. In certain embodiments, 1 to 20, tables are selected from Tables 5-1 to 5-20. In certain embodiments, 2 to 20, tables are selected from Tables 5-1 to 5-20. In certain embodiments, 2 to 4, 2 to 6, 2 to 8, 2 to 10, 2 to 12, 2 to 14, 2 to 15, 2 to 16, 2 to 18, 2 to 19, 2 to 20, 4 to 6, 4 to 8, 4 to 10, 4 to 12, 4 to 14, 4 to 15, 4 to 16, 4 to 18, 4 to 19, 4 to 20, 6 to 8, 6 to 10, 6 to 12, 6 to 14, 6 to 15, 6 to 16, 6 to 18, 6 to 19, 6 to 20, 8 to 10, 8 to 12, 8 to 14, 8 to 15, 8 to 16, 8 to 18, 8 to 19, 8 to 20, 10 to 12, 10 to 14, 10 to 15, 10 to 16, 10 to 18, 10 to 19, 10 to 20, 12 to 14, 12 to 15, 12 to 16, 12 to 18, 12 to 19, 12 to 20, 14 to 15, 14 to 16, 14 to 18, 14 to 19, 14 to 20, 15 to 16, 15 to 18, 15 to 19, 15 to 20, 16 to 18, 16 to 19, 16 to 20, 18 to 19, 18 to 20, or 19 to 20, tables are selected from Tables 5-1 to 5-20. In certain embodiments, at least 2, 4, 6, 8, 10, 12, 14, 15, 16, 18, or 19, tables are selected from Tables 5-1 to 5-20. In certain embodiments, 1, 2, 3, 4, 5, 6, 7, 8, 9, 10, 11, 12, 13, 14, 15, 16, 17, or 18, or any range there between tables are selected from Tables 5-1 to 5-4, 5-6 to 5-10, and 5-12 to 5-20. In certain embodiments, 1 to 18, tables are selected from Tables 5-1 to 5-4, 5-6 to 5-10, and 5-12 to 5-20. In certain embodiments, 2 to 18 tables are selected from Tables 5-1 to 5-4, 5-6 to 5-10, and 5-12 to 5-20. In certain embodiments, 2 to 4, 2 to 6, 2 to 8, 2 to 10, 2 to 12, 2 to 14, 2 to 15, 2 to 16, 2 to 18, 4 to 6, 4 to 8, 4 to 10, 4 to 12, 4 to 14, 4 to 15, 4 to 16, 4 to 18, 6 to 8, 6 to 10, 6 to 12, 6 to 14, 6 to 15, 6 to 16, 6 to 18, 8 to 10, 8 to 12, 8 to 14, 8 to 15, 8 to 16, 8 to 18, 10 to 12, 10 to 14, 10 to 15, 10 to 16, 10 to 18, 12 to 14, 12 to 15, 12 to 16, 12 to 18, 14 to 15, 14 to 16, 14 to 18, 15 to 16, 15 to 18, 16 to 18, or 17 to 18 tables are selected from Tables 5-1 to 5-4, 5-6 to 5-10, and 5-12 to 5-20. In certain embodiments, at least 2, 4, 6, 8, 10, 12, 14, 15, 16, or 17, tables are selected Tables 5-1 to 5-4, 5-6 to 5-10, and 5-12 to 5-20. The one or more GSVA scores of the patient can be generated based on comparing the gene expression measurements from the biological sample with a reference dataset. The reference dataset can be a reference dataset as described herein. The one or more GSVA scores of the patient can be generated using the input gene sets using a method described in the Examples, and/or as understood by a person of ordinary skill in the art.

In certain embodiments, the analyzing the data set comprises providing the dataset as an input to a machine learning model. The machine learning model can generate an inference indicative of the lupus disease state of the patient, based on the data set. The method can classify the lupus disease state of the patient based on the inference. In certain embodiments, the machine learning model generate the inference based on the one or more GSVA scores of the patient. In certain embodiments, the inference is whether the data set is indicative of the patient having lupus. In certain embodiments, the inference is whether the data set is indicative of the patient having active lupus, or inactive lupus. In certain embodiments, the inference is whether the data set is indicative of the patient having active lupus, inactive lupus, or not having lupus. In certain embodiments, the inference is whether the one or more GSVA scores of the patient, is indicative of the patient having lupus. In certain embodiments, the inference is whether the one or more GSVA scores of the patient, is indicative of the patient having active lupus, or inactive lupus. In certain embodiments, the inference is whether the one or more GSVA scores of the patient, is indicative of the patient having active lupus, inactive lupus, or not having lupus. The machine-learning model can be trained to generate the inference. In certain embodiments, the machine-learning model is (e.g., has been) trained to generate the inference of whether the data set is indicative of the patient having lupus. In certain embodiments, the machine-learning model is trained to generate the inference of whether the data set is indicative of the patient having active lupus, or inactive lupus. In certain embodiments, the machine-learning model is trained to generate the inference of whether the data set is indicative of the patient having active lupus, inactive lupus, or not having lupus. In certain embodiments, the machine-learning model is trained to generate the inference of whether the one or more GSVA scores of the patient, is indicative of the patient having lupus. In certain embodiments, the machine-learning model is trained to generate the inference of whether the one or more GSVA scores of the patient, is indicative of the patient having active lupus, or inactive lupus. In certain embodiments, the machine-learning model is trained to generate the inference of whether the one or more GSVA scores of the patient, is indicative of the patient having active lupus, inactive lupus, or not having lupus. In certain embodiments, the inference is that the data set is indicative of the patient having lupus, and the method classifies that the patient has lupus. In certain embodiments, the inference is that the data set is indicative of the patient does not have lupus, and the method classifies that the patient does not have lupus. In certain embodiments, the inference is that the data set is indicative of the patient having active lupus, and the method classifies that the patient has active lupus. In certain embodiments, the inference is that the data set is indicative of the patient having inactive lupus, and the method classifies that the patient has inactive lupus.

In certain embodiments, the method further comprises receiving, as an output of the machine-learning model, the inference; and/or electronically outputting a report indicative of the lupus disease state of the patient based on the inference.

The machine-learning model, can generate the inference, by comparing the data set to a reference data set. The reference data set can comprise and/or be derived from gene expression measurements from a plurality of reference biological samples. The plurality of reference biological samples can be obtained or derived from a plurality of reference subjects. In certain embodiments, a portion of the plurality of reference subjects do not have lupus. In certain embodiments, the plurality reference biological samples comprise a first plurality of reference biological samples obtained or derived from reference subjects not having lupus, and/or a second plurality of reference biological samples obtained or derived from reference subjects having lupus. In certain embodiments, the plurality reference biological samples comprise a first plurality of reference biological samples obtained or derived from reference subjects having active lupus, and/or a second plurality of reference biological samples obtained or derived from reference subjects having inactive lupus. In certain embodiments, the plurality reference biological samples comprise a first plurality of reference biological samples obtained or derived from reference subjects not having lupus, a second plurality of reference biological samples obtained or derived from reference subjects having active lupus, and/or a third plurality of reference biological samples obtained or derived from reference subjects having inactive lupus. In certain embodiments, the reference data set comprise and/or is derived from gene expression measurements from the plurality of reference biological samples of at least 2 genes selected from the genes listed in Table 3 and Tables 5-1 to 5-20. In certain embodiments, the reference data set comprise and/or is derived from gene expression measurements from the plurality of reference biological samples of at least 2 genes selected from the genes listed in Tables 5-1 to 5-20. In certain embodiments, the reference data set comprise and/or is derived from gene expression measurements from the plurality of reference biological samples of at least 2 genes selected from the genes listed in Tables 5-1 to 5-4, 5-6 to 5-10, and 5-11 to 5-20. In certain embodiments, the reference data set comprise a plurality of individual reference data sets, wherein a respective individual reference data set of the plurality of individual reference data sets, comprise and/or is derived from gene expression measurements of the at least 2 genes (e.g. the selected genes of the reference data set) from a reference biological sample of the plurality of reference biological samples. In certain embodiments, the reference data set comprise a plurality of individual reference data sets, wherein each individual reference data set of the plurality of individual reference data sets, comprise and/or is derived from gene expression measurements of the at least 2 genes (e.g. the selected genes of the reference data set) from a reference biological sample of the plurality of reference biological samples. Different individual reference data sets can be obtained from different reference biological samples. The selected genes of the dataset (e.g., gene expression measurements of which the dataset is comprised of or derived from), and the selected genes of the reference data set (e.g., gene expression measurements of which the reference dataset is comprised of or derived from) can at least partially overlap (e.g., one or more of the selected genes can be the same). In certain embodiments, the selected genes of the dataset, and the selected genes of the reference data are same. In certain embodiments, the selected genes of the dataset, and the selected genes of the reference data are same, and can be any selected genes set, e.g., of the data set, as described herein. In certain embodiments, the reference data set can be derived from the gene expression measurement data (e.g., of the selected genes of the reference data set) from the plurality of reference biological samples, wherein the gene expression measurement data is analyzed using a suitable data analysis tool including but not limited to a BIG-C™ big data analysis tool, an I-Scope™ big data analysis tool, a T-Scope™ big data analysis tool, a CellScan big data analysis tool, an MS (Molecular Signature) Scoring™ analysis tool, gene set variation analysis (GSVA), gene set enrichment analysis (GSEA), enrichment algorithm, Z score, multiscale embedded gene co-expression network analysis (MEGENA), weighted gene co-expression network analysis (WGCNA), differential expression analysis, log 2 expression analysis, or any combination thereof, to obtain the reference data set. In certain embodiments, the gene expression measurement data from the plurality of reference biological samples is analyzed using GSVA, to obtain the reference data set. In certain embodiments, the reference data set is obtained using GSVA, wherein the reference data set comprises one or more GSVA scores of the reference biological samples, wherein for a respective reference biological sample the one or more GSVA scores of the respective reference biological sample are generated based on one or more of the Tables selected from Tables 5-1 to 5-20, wherein for each selected Table, at least one GSVA score of the respective reference biological sample (e.g., of the reference subject from which the respective reference biological sample is derived from) is generated based on enrichment of expression of at least 2 genes listed in the respective selected Table, in the respective reference biological sample. The at least 2 genes from a respective selected Table, can form the input gene set for generating the at least one GSVA score based on the respective selected Table, using GSVA. In certain embodiments, one or more GSVA scores of each reference biological samples (and/or of the each of the reference subjects) are generated, wherein the one or more GSVA scores of different reference biological samples can be same or different. The enrichment of expression of the at least 2 genes in a respective reference biological sample, can be measured by comparing gene expression measurements data of the respective reference biological sample, with the gene expression measurements data of the reference biological samples (e.g., cohort). The one or more GSVA scores of the patient can be generated based on comparing the gene expression measurements from the biological sample from the patient with the gene expression measurements from the reference dataset. In certain embodiments, the one or more Tables are selected from Tables 5-1 to 5-4, 5-6 to 5-10, and 5-12 to 5-20.

The machine learning model can be trained (e.g., can be obtained by training) with the reference data set. In certain embodiments, the reference data set comprises the plurality of individual reference data sets. The plurality of individual reference data sets, can be obtained from the plurality of reference subjects. Different individual reference data sets can be obtained from different reference subjects. A respective individual reference data set can comprise or is derived from gene expression measurements (e.g., of the selected genes of the reference data set), from a respective reference biological sample obtained or derived from a respective reference subject. In certain embodiments, each individual reference data set can comprise or is derived from gene expression measurements (e.g., of the selected genes of the reference data set), from a reference biological sample obtained or derived from a reference subject, wherein different individual reference data sets are obtained from different reference subjects. In certain embodiments, oversampling or undersampling correction is made during training of the machine learning model. For example, if a reference data set includes a greater number of samples identified as having lupus and a relatively fewer number of samples identified as healthy control, the healthy controls may be oversampled to produce a data set that has equal number of lupus samples and control samples. The machine learning model can be trained to infer the lupus disease state of a reference subject based on the individual reference data set from the reference subject. The machine learning model can be trained using a suitable method, and a suitable reference data set such that the machine learning model (e.g., obtained by training) can generate the inference indicative of the lupus disease state of the patient based on the data set, with a desired accuracy, sensitivity, specificity, positive predictive value, and/or negative predictive value. The desired accuracy, sensitivity, specificity, positive predictive value, and/or negative predictive value, can be respectively an accuracy, sensitivity, specificity, positive predictive value, and/or negative predictive value described herein. The individual reference data set can be an individual reference data set as described herein. The suitable method can be a training method as described in the Example, and/or the suitable reference dataset can be dataset as described in the Example. In certain embodiments, a first portion of the reference data set can be used as training data set, and a second portion of the reference data set can be used as validation data set, for training the machine learning model. In certain embodiments, 0 to 25 fold, such as 0, 1, 2, 3, 4, 5, 6, 7, 8, 9, 10, 11, 12, 13, 14, 15, 16, 17, 18, 19, 20, 21, 22, 23, 24 or 25 fold validation is used. In certain embodiments, 6 fold validation is used. In certain embodiments, 10 fold validation is used. In the certain embodiments, the machine-learning model generate the inference based on the one or more GSVA scores of the patient, and the machine-learning model is trained with a reference dataset comprising one or more GSVA scores from the plurality of reference biological samples. The one or more GSVA scores of the patient can be generated based on comparing the data set with a reference data set as described herein. In certain embodiments, the one or more GSVA scores of the patient are generated based on comparing the data set with the reference data set, and the enrichment of expression of genes, (e.g., for calculating the one or more GSVA scores of the patient) in the biological sample from the patient can be measured by comparing gene expression measurements from the biological sample from the patient, with the gene expression measurements from the plurality of reference biological samples of the reference data set. The reference data set used for generating the one or more GSVA scores of the patient, and the reference data set used for training the machine learning model can be the same or different.

The machine-learning model can be trained (e.g., obtained by training) using linear regression, logistic regression (LOG), Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, a Random Forest (RF), deep learning algorithm, linear discriminant analysis (LDA), decision tree learning (DTREE), adaptive boosting (ADB), Classification and Regression Tree (CART), hierarchical clustering, or any combination thereof. The algorithm of the machine learning model can be the machine learning classifiers, e.g., mentioned in this paragraph. The machine learning classifiers (e.g., linear regression, LOG, Ridge regression, Lasso regression, EN regression, SVM, GBM, kNN, GLM, NB classifier, neural network, a RF, deep learning algorithm, LDA, DTREE, ADB, CART, and/or hierarchical clustering) can be trained to obtain the machine learning model. The machine learning classifier can be a supervised machine learning algorithm or an unsupervised machine learning algorithm. In certain embodiments, the machine learning model is trained using linear regression. In certain embodiments, the machine learning model is trained using LOG. In certain embodiments, the machine learning model is trained using Ridge regression. In certain embodiments, the machine learning model is trained using Lasso regression. In certain embodiments, the machine learning model is trained using EN. In certain embodiments, the machine learning model is trained using SVM. In certain embodiments, the machine learning model is trained using GBM. In certain embodiments, the machine learning model is trained using KNN. In certain embodiments, the machine learning model is trained using GLM. In certain embodiments, the machine learning model is trained using NB. In certain embodiments, the machine learning model is trained using RF. In certain embodiments, the machine learning model is trained using deep learning algorithm. In certain embodiments, the machine learning model is trained using LDA. In certain embodiments, the machine learning model is trained using DTREE. In certain embodiments, the machine learning model is trained using ADB. In certain embodiments, the machine learning model is trained using CART. In some embodiments, the machine learning model, is trained using a supervised machine learning algorithm. In some embodiments, the machine learning model, is trained using an unsupervised machine learning algorithm.

The reference biological sample can contain a blood sample, isolated peripheral blood mononuclear cells (PBMCs), tissue biopsy sample, nasal fluid, saliva, urine, stool, or any derivative thereof. In certain embodiments, the reference biological sample contains a blood sample or any derivative thereof. In certain embodiments, the reference biological sample contains PBMCs or any derivative thereof. In certain embodiments, the reference biological sample contains a tissue biopsy sample or any derivative thereof. The reference subjects can be humans.

In certain embodiments, analyzing the data set comprises developing a risk score for the patient based at least on the data set, and classifying the lupus disease state of the patient based at least on the risk score of the patient. In certain embodiments, the risk score for the patient is developed based on the enrichment score, such as one or more GSVA scores, of the patient.

In certain embodiments, the method classify the lupus disease state of the patient with an accuracy of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In certain embodiments, the method classify the lupus disease state of the patient with an accuracy of about 65% to about 100%. In certain embodiments, the method classify the lupus disease state of the patient with an accuracy of about 65% to about 70%, about 65% to about 75%, about 65% to about 80%, about 65% to about 85%, about 65% to about 90%, about 65% to about 93%, about 65% to about 95%, about 65% to about 97%, about 65% to about 98%, about 65% to about 99%, about 65% to about 100%, about 70% to about 75%, about 70% to about 80%, about 70% to about 85%, about 70% to about 90%, about 70% to about 93%, about 70% to about 95%, about 70% to about 97%, about 70% to about 98%, about 70% to about 99%, about 70% to about 100%, about 75% to about 80%, about 75% to about 85%, about 75% to about 90%, about 75% to about 93%, about 75% to about 95%, about 75% to about 97%, about 75% to about 98%, about 75% to about 99%, about 75% to about 100%, about 80% to about 85%, about 80% to about 90%, about 80% to about 93%, about 80% to about 95%, about 80% to about 97%, about 80% to about 98%, about 80% to about 99%, about 80% to about 100%, about 85% to about 90%, about 85% to about 93%, about 85% to about 95%, about 85% to about 97%, about 85% to about 98%, about 85% to about 99%, about 85% to about 100%, about 90% to about 93%, about 90% to about 95%, about 90% to about 97%, about 90% to about 98%, about 90% to about 99%, about 90% to about 100%, about 93% to about 95%, about 93% to about 97%, about 93% to about 98%, about 93% to about 99%, about 93% to about 100%, about 95% to about 97%, about 95% to about 98%, about 95% to about 99%, about 95% to about 100%, about 97% to about 98%, about 97% to about 99%, about 97% to about 100%, about 98% to about 99%, about 98% to about 100%, or about 99% to about 100%. In certain embodiments, the method classify the lupus disease state of the patient with an accuracy of about 65%, about 70%, about 75%, about 80%, about 85%, about 90%, about 93%, about 95%, about 97%, about 98%, about 99%, or about 100%. In certain embodiments, the method classify the lupus disease state of the patient with an accuracy of at least about 65%, about 70%, about 75%, about 80%, about 85%, about 90%, about 93%, about 95%, about 97%, about 98%, or about 99%.

In certain embodiments, the method classify the lupus disease state of the patient with a sensitivity of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In certain embodiments, the method classify the lupus disease state of the patient with a sensitivity of about 65% to about 100%. In certain embodiments, the method classify the lupus disease state of the patient with a sensitivity of about 65% to about 70%, about 65% to about 75%, about 65% to about 80%, about 65% to about 85%, about 65% to about 90%, about 65% to about 93%, about 65% to about 95%, about 65% to about 97%, about 65% to about 98%, about 65% to about 99%, about 65% to about 100%, about 70% to about 75%, about 70% to about 80%, about 70% to about 85%, about 70% to about 90%, about 70% to about 93%, about 70% to about 95%, about 70% to about 97%, about 70% to about 98%, about 70% to about 99%, about 70% to about 100%, about 75% to about 80%, about 75% to about 85%, about 75% to about 90%, about 75% to about 93%, about 75% to about 95%, about 75% to about 97%, about 75% to about 98%, about 75% to about 99%, about 75% to about 100%, about 80% to about 85%, about 80% to about 90%, about 80% to about 93%, about 80% to about 95%, about 80% to about 97%, about 80% to about 98%, about 80% to about 99%, about 80% to about 100%, about 85% to about 90%, about 85% to about 93%, about 85% to about 95%, about 85% to about 97%, about 85% to about 98%, about 85% to about 99%, about 85% to about 100%, about 90% to about 93%, about 90% to about 95%, about 90% to about 97%, about 90% to about 98%, about 90% to about 99%, about 90% to about 100%, about 93% to about 95%, about 93% to about 97%, about 93% to about 98%, about 93% to about 99%, about 93% to about 100%, about 95% to about 97%, about 95% to about 98%, about 95% to about 99%, about 95% to about 100%, about 97% to about 98%, about 97% to about 99%, about 97% to about 100%, about 98% to about 99%, about 98% to about 100%, or about 99% to about 100%. In certain embodiments, the method classify the lupus disease state of the patient with a sensitivity of about 65%, about 70%, about 75%, about 80%, about 85%, about 90%, about 93%, about 95%, about 97%, about 98%, about 99%, or about 100%. In certain embodiments, the method classify the lupus disease state of the patient with a sensitivity of at least about 65%, about 70%, about 75%, about 80%, about 85%, about 90%, about 93%, about 95%, about 97%, about 98%, or about 99%.

In certain embodiments, the method classify the lupus disease state of the patient with a specificity of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In certain embodiments, the method classify the lupus disease state of the patient with a specificity of about 65% to about 100%. In certain embodiments, the method classify the lupus disease state of the patient with a specificity of about 65% to about 70%, about 65% to about 75%, about 65% to about 80%, about 65% to about 85%, about 65% to about 90%, about 65% to about 93%, about 65% to about 95%, about 65% to about 97%, about 65% to about 98%, about 65% to about 99%, about 65% to about 100%, about 70% to about 75%, about 70% to about 80%, about 70% to about 85%, about 70% to about 90%, about 70% to about 93%, about 70% to about 95%, about 70% to about 97%, about 70% to about 98%, about 70% to about 99%, about 70% to about 100%, about 75% to about 80%, about 75% to about 85%, about 75% to about 90%, about 75% to about 93%, about 75% to about 95%, about 75% to about 97%, about 75% to about 98%, about 75% to about 99%, about 75% to about 100%, about 80% to about 85%, about 80% to about 90%, about 80% to about 93%, about 80% to about 95%, about 80% to about 97%, about 80% to about 98%, about 80% to about 99%, about 80% to about 100%, about 85% to about 90%, about 85% to about 93%, about 85% to about 95%, about 85% to about 97%, about 85% to about 98%, about 85% to about 99%, about 85% to about 100%, about 90% to about 93%, about 90% to about 95%, about 90% to about 97%, about 90% to about 98%, about 90% to about 99%, about 90% to about 100%, about 93% to about 95%, about 93% to about 97%, about 93% to about 98%, about 93% to about 99%, about 93% to about 100%, about 95% to about 97%, about 95% to about 98%, about 95% to about 99%, about 95% to about 100%, about 97% to about 98%, about 97% to about 99%, about 97% to about 100%, about 98% to about 99%, about 98% to about 100%, or about 99% to about 100%. In certain embodiments, the method classify the lupus disease state of the patient with a specificity of about 65%, about 70%, about 75%, about 80%, about 85%, about 90%, about 93%, about 95%, about 97%, about 98%, about 99%, or about 100%. In certain embodiments, the method classify the lupus disease state of the patient with a specificity of at least about 65%, about 70%, about 75%, about 80%, about 85%, about 90%, about 93%, about 95%, about 97%, about 98%, or about 99%.

In certain embodiments, the method classify the lupus disease state of the patient with a positive predictive value of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In certain embodiments, the method classify the lupus disease state of the patient with a positive predictive value of about 65% to about 100%. In certain embodiments, the method classify the lupus disease state of the patient with a positive predictive value of about 65% to about 70%, about 65% to about 75%, about 65% to about 80%, about 65% to about 85%, about 65% to about 90%, about 65% to about 93%, about 65% to about 95%, about 65% to about 97%, about 65% to about 98%, about 65% to about 99%, about 65% to about 100%, about 70% to about 75%, about 70% to about 80%, about 70% to about 85%, about 70% to about 90%, about 70% to about 93%, about 70% to about 95%, about 70% to about 97%, about 70% to about 98%, about 70% to about 99%, about 70% to about 100%, about 75% to about 80%, about 75% to about 85%, about 75% to about 90%, about 75% to about 93%, about 75% to about 95%, about 75% to about 97%, about 75% to about 98%, about 75% to about 99%, about 75% to about 100%, about 80% to about 85%, about 80% to about 90%, about 80% to about 93%, about 80% to about 95%, about 80% to about 97%, about 80% to about 98%, about 80% to about 99%, about 80% to about 100%, about 85% to about 90%, about 85% to about 93%, about 85% to about 95%, about 85% to about 97%, about 85% to about 98%, about 85% to about 99%, about 85% to about 100%, about 90% to about 93%, about 90% to about 95%, about 90% to about 97%, about 90% to about 98%, about 90% to about 99%, about 90% to about 100%, about 93% to about 95%, about 93% to about 97%, about 93% to about 98%, about 93% to about 99%, about 93% to about 100%, about 95% to about 97%, about 95% to about 98%, about 95% to about 99%, about 95% to about 100%, about 97% to about 98%, about 97% to about 99%, about 97% to about 100%, about 98% to about 99%, about 98% to about 100%, or about 99% to about 100%. In certain embodiments, the method classify the lupus disease state of the patient with a positive predictive value of about 65%, about 70%, about 75%, about 80%, about 85%, about 90%, about 93%, about 95%, about 97%, about 98%, about 99%, or about 100%. In certain embodiments, the method classify the lupus disease state of the patient with a positive predictive value of at least about 65%, about 70%, about 75%, about 80%, about 85%, about 90%, about 93%, about 95%, about 97%, about 98%, or about 99%.

In certain embodiments, the method classify the lupus disease state of the patient with a negative predictive value of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In certain embodiments, the method classify the lupus disease state of the patient with a negative predictive value of about 65% to about 100%. In certain embodiments, the method classify the lupus disease state of the patient with a negative predictive value of about 65% to about 70%, about 65% to about 75%, about 65% to about 80%, about 65% to about 85%, about 65% to about 90%, about 65% to about 93%, about 65% to about 95%, about 65% to about 97%, about 65% to about 98%, about 65% to about 99%, about 65% to about 100%, about 70% to about 75%, about 70% to about 80%, about 70% to about 85%, about 70% to about 90%, about 70% to about 93%, about 70% to about 95%, about 70% to about 97%, about 70% to about 98%, about 70% to about 99%, about 70% to about 100%, about 75% to about 80%, about 75% to about 85%, about 75% to about 90%, about 75% to about 93%, about 75% to about 95%, about 75% to about 97%, about 75% to about 98%, about 75% to about 99%, about 75% to about 100%, about 80% to about 85%, about 80% to about 90%, about 80% to about 93%, about 80% to about 95%, about 80% to about 97%, about 80% to about 98%, about 80% to about 99%, about 80% to about 100%, about 85% to about 90%, about 85% to about 93%, about 85% to about 95%, about 85% to about 97%, about 85% to about 98%, about 85% to about 99%, about 85% to about 100%, about 90% to about 93%, about 90% to about 95%, about 90% to about 97%, about 90% to about 98%, about 90% to about 99%, about 90% to about 100%, about 93% to about 95%, about 93% to about 97%, about 93% to about 98%, about 93% to about 99%, about 93% to about 100%, about 95% to about 97%, about 95% to about 98%, about 95% to about 99%, about 95% to about 100%, about 97% to about 98%, about 97% to about 99%, about 97% to about 100%, about 98% to about 99%, about 98% to about 100%, or about 99% to about 100%. In certain embodiments, the method classify the lupus disease state of the patient with a negative predictive value of about 65%, about 70%, about 75%, about 80%, about 85%, about 90%, about 93%, about 95%, about 97%, about 98%, about 99%, or about 100%. In certain embodiments, the method classify the lupus disease state of the patient with a negative predictive value of at least about 65%, about 70%, about 75%, about 80%, about 85%, about 90%, about 93%, about 95%, about 97%, about 98%, or about 99%.

The machine-learning model can have the accuracy, sensitivity, specificity, positive predictive value, and/or negative predictive value, as described above, and the accuracy, sensitivity, specificity, positive predictive value, and negative predictive value, value of the method can be based on the classification parameters of the machine-learning model, as described herein and/or as understood by one of skill in the art.

In certain embodiments, the machine learning model has a receiver operating characteristic (ROC) curve with an Area-Under-Curve (AUC) of at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. In certain embodiments, the AUC of the ROC is about 0.65 to about 1. In certain embodiments, the AUC of the ROC is about 0.65 to about 0.7, about 0.65 to about 0.75, about 0.65 to about 0.8, about 0.65 to about 0.85, about 0.65 to about 0.9, about 0.65 to about 0.93, about 0.65 to about 0.95, about 0.65 to about 0.97, about 0.65 to about 0.98, about 0.65 to about 0.99, about 0.65 to about 1, about 0.7 to about 0.75, about 0.7 to about 0.8, about 0.7 to about 0.85, about 0.7 to about 0.9, about 0.7 to about 0.93, about 0.7 to about 0.95, about 0.7 to about 0.97, about 0.7 to about 0.98, about 0.7 to about 0.99, about 0.7 to about 1, about 0.75 to about 0.8, about 0.75 to about 0.85, about 0.75 to about 0.9, about 0.75 to about 0.93, about 0.75 to about 0.95, about 0.75 to about 0.97, about 0.75 to about 0.98, about 0.75 to about 0.99, about 0.75 to about 1, about 0.8 to about 0.85, about 0.8 to about 0.9, about 0.8 to about 0.93, about 0.8 to about 0.95, about 0.8 to about 0.97, about 0.8 to about 0.98, about 0.8 to about 0.99, about 0.8 to about 1, about 0.85 to about 0.9, about 0.85 to about 0.93, about 0.85 to about 0.95, about 0.85 to about 0.97, about 0.85 to about 0.98, about 0.85 to about 0.99, about 0.85 to about 1, about 0.9 to about 0.93, about 0.9 to about 0.95, about 0.9 to about 0.97, about 0.9 to about 0.98, about 0.9 to about 0.99, about 0.9 to about 1, about 0.93 to about 0.95, about 0.93 to about 0.97, about 0.93 to about 0.98, about 0.93 to about 0.99, about 0.93 to about 1, about 0.95 to about 0.97, about 0.95 to about 0.98, about 0.95 to about 0.99, about 0.95 to about 1, about 0.97 to about 0.98, about 0.97 to about 0.99, about 0.97 to about 1, about 0.98 to about 0.99, about 0.98 to about 1, or about 0.99 to about 1. In certain embodiments, the AUC of the ROC is about 0.65, about 0.7, about 0.75, about 0.8, about 0.85, about 0.9, about 0.93, about 0.95, about 0.97, about 0.98, about 0.99, or about 1. In certain embodiments, the AUC of the ROC is at least about 0.65, about 0.7, about 0.75, about 0.8, about 0.85, about 0.9, about 0.93, about 0.95, about 0.97, about 0.98, or about 0.99.

The inference can have a confidence value between 0 and 1. In certain embodiments, the confidence value of the inference is between 0 and 1, such as, 0, 0.1, 0.2, 0.3, 0.4, 0.5, 0.6, 0.7, 0.8, 0.9 or 1, or any value or ranges there between, that the patient has lupus. In certain embodiments, the confidence value of the inference is between 0 and 1, such as, 0, 0.1, 0.2, 0.3, 0.4, 0.5, 0.6, 0.7, 0.8, 0.9 or 1, or any value or ranges there between, that the patient has active lupus. In certain embodiments, the confidence value of the inference is between 0 and 1, such as, 0, 0.1, 0.2, 0.3, 0.4, 0.5, 0.6, 0.7, 0.8, 0.9 or 1, or any value or ranges there between, that the patient has inactive lupus.

The biological sample can be obtained or derived from the patient. The biological sample can contain a blood sample, isolated peripheral blood mononuclear cells (PBMCs), tissue biopsy sample, nasal fluid, saliva, urine, stool, or any derivative thereof. In certain embodiments, the biological sample contains a blood sample or any derivative thereof. In certain embodiments, the biological sample contains PBMCs or any derivative thereof. In certain embodiments, the biological sample contains a tissue biopsy sample or any derivative thereof. In certain embodiments, the biological sample contains a nasal fluid sample or any derivative thereof. In certain embodiments, the biological sample contains a saliva sample or any derivative thereof. In certain embodiments, the biological sample contains a urine sample or any derivative thereof. In certain embodiments, the biological sample contains a stool sample or any derivative thereof. The patient can be a human patient.

In certain embodiments, the method further comprises monitoring the lupus disease state of the patient, wherein the monitoring comprises assessing (e.g., classifying) the lupus disease state of the patient at a plurality of different time points. A difference in the assessment of the lupus disease state of the patient among the plurality of time points can be indicative of one or more clinical indications selected from the group consisting of: (i) a diagnosis of the lupus disease state of the patient, (ii) a prognosis of the lupus disease state of the patient, and (iii) an efficacy or non-efficacy of a course of treatment for treating the lupus disease state of the patient. In certain embodiments, the patient has been administered a treatment, and the method can assess an efficacy or non-efficacy of the treatment, for treating the lupus disease state of the patient.

In certain embodiments, the data set comprises or is derived from gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 140, 150, or all, or any range or value there between genes selected from genes listed in Table 5-16, Table 5-15, Table 5-18, and Table 5-10, from the biological sample from the patient, and the dataset is analyzed to classify whether the patient has lupus. In certain embodiments, the data set comprises or is derived from gene expression measurements of the genes listed in Table 5-16, Table 5-15, Table 5-18, and Table 5-10, from the biological sample from the patient, and the dataset is analyzed to classify whether the patient has lupus. In certain embodiments, the data set comprises or is derived from gene expression measurements of at least 2 genes selected from each of 1, 2, 3, or 4, or 1 to 4, or 2 to 4, or 3 or 4 or any range there between, Tables selected from Table 5-16, Table 5-15, Table 5-18, and Table 5-10, from the biological sample from the patient, and the dataset is analyzed to classify whether the patient has lupus. In certain embodiments, the data set comprises or is derived from gene expression measurements of at least 2 genes selected from each of Table 5-16, Table 5-15, Table 5-18, and Table 5-10, from the biological sample from the patient, and the dataset is analyzed to classify whether the patient has lupus. In certain embodiments, for each selected Table (e.g., from Table 5-16, Table 5-15, Table 5-18, and Table 5-10), the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, or 135, or all genes selected from the genes listed in the selected Table, from the biological sample from the patient, and the dataset is analyzed to classify whether the patient has lupus, wherein the number of genes selected from different selected tables are the same or different. In certain embodiments, for each selected Table (e.g., from Table 5-16, Table 5-15, Table 5-18, and Table 5-10), the data set comprises or is derived from gene expression measurements of an effective number of genes selected from the genes listed in the selected Table, from the biological sample from the patient, wherein the number of genes selected from different selected Tables are the same or different and wherein the dataset is analyzed to classify whether the patient has lupus. In certain embodiments, the data set comprises or is derived from gene expression measurements of an effective number of genes selected from genes listed in each of 1, 2, 3, or 4, or 1 to 4, or 2 to 4, or 3 or 4 or any range there between, Tables selected from Table 5-16, Table 5-15, Table 5-18, and Table 5-10, from the biological sample from the patient, wherein the number of genes selected from different selected Tables are the same or different, and wherein the dataset is analyzed to classify whether the patient has lupus. In certain embodiments, the data set comprises or is derived from gene expression measurements of an effective number of genes selected from genes listed in each Tables selected from Table 5-16, Table 5-15, Table 5-18, and Table 5-10, from the biological sample from the patient, (i.e., the data set comprises or is derived from gene expression measurements of an effective number of genes selected from the genes listed in Table 5-16; an effective number of genes selected from the genes listed in Table 5-15; an effective number of genes selected from the genes listed in Table 5-18; and an effective number of genes selected from the genes listed in Table 5-10; from the biological sample from the patient), wherein the number of genes selected from different selected Tables are the same or different, and wherein the dataset is analyzed to classify whether the patient has lupus. In certain embodiments, the data set comprises or is derived from gene expression measurements of the genes listed in each of 1, 2, 3, or 4, or 1 to 4, or 2 to 4, or 3 or 4 or any range there between, Tables selected from Table 5-16, Table 5-15, Table 5-18, and Table 5-10, from the biological sample from the patient, and wherein the dataset is analyzed to classify whether the patient has lupus. In certain embodiments, the one or more GSVA scores of the patient are generated based on 1, 2, 3 or 4 Tables selected from Table 5-16, Table 5-15, Table 5-18, and Table 5-10, and the one or more GSVA scores are analyzed to classify whether the patient has lupus. In certain embodiments, Table 5-16, Table 5-15, Table 5-18, and Table 5-10 are selected, and the one or more GSVA scores of the patient comprises at least 4 GSVA scores (e.g., at least 1 GSVA score based on Table 5-16, at least 1 GSVA score based on Table 5-15, at least 1 GSVA score based on Table 5-18, and at least 1 GSVA score based on Table 5-10), and the one or more GSVA scores are analyzed to classify whether the patient has lupus. In certain embodiments, the one or more GSVA scores of the patient are generated based on 1, 2, 3 or 4 Tables selected from Table 5-16, Table 5-15, Table 5-18, and Table 5-10, wherein for each selected Table, one GSVA score is generated, and the one or more GSVA scores are analyzed to classify whether the patient has lupus. In certain embodiments, Table 5-16, Table 5-15, Table 5-18, and Table 5-10 are selected, and for each selected Table one GSVA score is generated, and the one or more GSVA scores of the patient comprises 4 GSVA scores (e.g., 1 GSVA score based on Table 5-16, 1 GSVA score based on Table 5-15, 1 GSVA score based on Table 5-18, and 1 GSVA score based on Table 5-10), and the one or more GSVA scores are analyzed to classify whether the patient has lupus. For a selected Table the GSVA score(s) based on the selected Table can be generated using an input gene set as described herein. In certain embodiments, the inference of the machine learning model is, whether the data set (e.g., a data set mentioned in this paragraph) is indicative of the patient having lupus. In certain embodiments, the confidence value of the inference of the machine learning model is between 0 and 1, such as, 0, 0.1, 0.2, 0.3, 0.4, 0.5, 0.6, 0.7, 0.8, 0.9 or 1, or any value or ranges there between, that the patient has lupus.

In certain embodiments, the data set comprises or is derived from gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 140, 150, or all, or any range or value there between genes selected from genes listed in Table 5-20, Table 5-19, Table 5-4, and Table 5-17, from the biological sample from the patient, and the dataset is analyzed to classify whether the patient has active lupus, or inactive lupus. In certain embodiments, the data set comprises or is derived from gene expression measurements of the genes listed in Table 5-20, Table 5-19, Table 5-4, and Table 5-17, from the biological sample from the patient, and the dataset is analyzed to classify whether the patient has active lupus, or inactive lupus. In certain embodiments, the data set comprises or is derived from gene expression measurements of at least 2 genes selected from each of 1, 2, 3, or 4, or 1 to 4, or 2 to 4, or 3 or 4 or any range there between, Tables selected from Table 5-20, Table 5-19, Table 5-4, and Table 5-17, from the biological sample from the patient, and the dataset is analyzed to classify whether the patient has active lupus, or inactive lupus. In certain embodiments, the data set comprises or is derived from gene expression measurements of at least 2 genes selected from each of Table 5-20, Table 5-19, Table 5-4, and Table 5-17, from the biological sample from the patient, and the dataset is analyzed to classify whether the patient has active lupus, or inactive lupus. In certain embodiments, for each selected Table (e.g., from Table 5-20, Table 5-19, Table 5-4, and Table 5-17), the data set comprises or is derived from gene expression measurements of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, or 135, or all genes selected from the genes listed in the selected Table, from the biological sample from the patient, and the dataset is analyzed to classify whether the patient has active lupus, or inactive lupus, wherein the number of genes selected from different selected tables are the same or different. In certain embodiments, for each selected Table (e.g., from Table 5-20, Table 5-19, Table 5-4, and Table 5-17), the data set comprises or is derived from gene expression measurements of an effective number of genes selected from the genes listed in the selected Table, from the biological sample from the patient, wherein the number of genes selected from different selected Tables are the same or different and wherein the dataset is analyzed to classify whether the patient has active lupus, or inactive lupus. In certain embodiments, the data set comprises or is derived from gene expression measurements of an effective number of genes selected from genes listed in each of 1, 2, 3, or 4, or 1 to 4, or 2 to 4, or 3 or 4 or any range there between, Tables selected from Table 5-20, Table 5-19, Table 5-4, and Table 5-17, from the biological sample from the patient, wherein the number of genes selected from different selected Tables are the same or different, and wherein the dataset is analyzed to classify whether the patient has active lupus, or inactive lupus. In certain embodiments, the data set comprises or is derived from gene expression measurements of an effective number of genes selected from genes listed in each Tables selected from Table 5-20, Table 5-19, Table 5-4, and Table 5-17, from the biological sample from the patient, (i.e., the data set comprises or is derived from gene expression measurements of an effective number of genes selected from the genes listed in Table 5-20; an effective number of genes selected from the genes listed in Table 5-19; an effective number of genes selected from the genes listed in Table 5-4; and an effective number of genes selected from the genes listed in Table 5-17; from the biological sample from the patient), wherein the number of genes selected from different selected Tables are the same or different, and wherein the dataset is analyzed to classify whether the patient has active lupus, or inactive lupus. In certain embodiments, the data set comprises or is derived from gene expression measurements of the genes listed in each of 1, 2, 3, or 4, or 1 to 4, or 2 to 4, or 3 or 4 or any range there between, Tables selected from Table 5-20, Table 5-19, Table 5-4, and Table 5-17, from the biological sample from the patient, and wherein the dataset is analyzed to classify whether the patient has active lupus, or inactive lupus. In certain embodiments, the one or more GSVA scores of the patient are generated based on 1, 2, 3 or 4 Tables selected from Table 5-20, Table 5-19, Table 5-4, and Table 5-17, and the one or more GSVA scores are analyzed to classify whether the patient has active lupus, or inactive lupus. In certain embodiments, Table 5-20, Table 5-19, Table 5-4, and Table 5-17 are selected, and the one or more GSVA scores of the patient comprises at least 4 GSVA scores (e.g., at least 1 GSVA score based on Table 5-20, at least 1 GSVA score based on Table 5-19, at least 1 GSVA score based on Table 5-4, and at least 1 GSVA score based on Table 5-17), and the one or more GSVA scores are analyzed to classify whether the patient has active lupus, or inactive lupus. In certain embodiments, the one or more GSVA scores of the patient are generated based on 1, 2, 3 or 4 Tables selected from Table 5-20, Table 5-19, Table 5-4, and Table 5-17, wherein for each selected Table, one GSVA score is generated, and the one or more GSVA scores are analyzed to classify whether the patient has active lupus, or inactive lupus. In certain embodiments, Table 5-20, Table 5-19, Table 5-4, and Table 5-17 are selected, and for each selected Table one GSVA score is generated, and the one or more GSVA scores of the patient comprises 4 GSVA scores (e.g., 1 GSVA score based on Table 5-20, 1 GSVA score based on Table 5-19, 1 GSVA score based on Table 5-4, and 1 GSVA score based on Table 5-17), and the one or more GSVA scores are analyzed to classify whether the patient has active lupus, or inactive lupus. For a selected Table the GSVA score(s) based on the selected Table can be generated using an input gene set as described herein. In certain embodiments, the inference of the machine learning model is, whether the data set (e.g., a data set mentioned in this paragraph) is indicative of the patient having active lupus, or inactive lupus. In certain embodiments, the confidence value of the inference of the machine learning model is between 0 and 1, such as, 0, 0.1, 0.2, 0.3, 0.4, 0.5, 0.6, 0.7, 0.8, 0.9 or 1, or any value or ranges there between, that the patient has active lupus. In certain embodiments, the confidence value of the inference of the machine learning model is between 0 and 1, such as, 0, 0.1, 0.2, 0.3, 0.4, 0.5, 0.6, 0.7, 0.8, 0.9 or 1, or any value or ranges there between, that the patient has inactive lupus.

The patient can be a human patient. In certain embodiments, the patient has lupus. In certain embodiments, the patient is asymptomatic of lupus. In certain embodiments, the patient is suspected of having lupus. In certain embodiments, the patient has active lupus. In certain embodiments, the patient is suspected of having active lupus. In certain embodiments, the patient has inactive lupus. In certain embodiments, the patient is suspected of having inactive lupus.

In certain embodiments, the method further comprises administering a treatment to the patient. In certain embodiments, the treatment is administered based on the determination that the patient has lupus. In certain embodiments, the treatment is administered based on the determination that the patient has active lupus. In certain embodiments, the treatment is configured to treat lupus. In certain embodiments, the treatment is configured to reduce severity of lupus. In certain embodiments, the treatment is configured to reduce a risk of having lupus. In certain embodiments, the treatment is configured to treat active lupus. In certain embodiments, the treatment is configured to reduce severity of active lupus. In certain embodiments, the treatment is configured to reduce a risk of having active lupus. In certain embodiments, the treatment is configured to treat inactive lupus. In certain embodiments, the treatment is configured to reduce severity of inactive lupus. In certain embodiments, the treatment is configured to reduce a risk of having inactive lupus.

In certain embodiments, the treatment for lupus, and/or active lupus comprises a neutrophil function inhibitor, a TNF inhibitor, an IL1 inhibitor, a Plasma cell inhibitor, a NK cell inhibitor, a B Cell Inhibitor, or any combination thereof. Non-limiting examples of an IFN inhibitor include Anifrolumab. Non-limiting examples of a Plasma cell inhibitor include Mycophenolate, Bortezomib, Carfilzomib, Ixazomib, Daratumumab, Isatuximab and Elotuzumab. Non-limiting examples of an IL1 inhibitor include Anakinra, and Canakinumab. Non-limiting examples of a TNF inhibitor include Adalimumab, Certolizumab pegol, Etanercept, Golimumab, and Infliximab. Non-limiting examples of a Neutrophil function inhibitor include Dasatinib, Apremilast, and Roflumilast. Non-limiting examples of a NK cell inhibitor include Azathioprine. Non-limiting examples of a B cell inhibitor include Belimumab, Rituximab, Obinutuzumab, and Inebilizumab. In certain embodiments, the treatment for lupus, and/or active lupus comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Inebilizumab, or any combination thereof.

Certain aspects, are directed to a biomarker assay developed according to a method described herein. Certain aspects, are directed to a kit comprising the biomarker assay developed according to a method described herein, and/or a biomarker assay of described herein.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for identifying one or more records having a specific phenotype. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Biological Data Analysis

Certain embodiments, of the present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof.

In an aspect, the present disclosure provides a computer-implemented method for assessing a condition of a subject, comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject.

In some embodiments, the dataset comprises mRNA gene expression or transcriptome data, DNA genomic data, proteomic data, metabolomic data, or a combination thereof. In some embodiments, the biological sample is selected from the group consisting of: a whole blood (WB) sample, a PBMC sample, a tissue sample, and a cell sample. In some embodiments, assessing the condition of the subject comprises identifying a disease or disorder of the subject.

In some embodiments, the method further comprises identifying a disease or disorder of the subject at a sensitivity or specificity of at least about 70%. In some embodiments, the method further comprises determining a likelihood of the identification of the disease or disorder of the subject. In some embodiments, the method further comprises providing a therapeutic intervention for the disease or disorder of the subject. In some embodiments, the method further comprises monitoring the disease or disorder of the subject, wherein the monitoring comprises assessing the disease or disorder of the subject at a plurality of time points, wherein the assessing is based at least on the disease or disorder identified at each of the plurality of time points.

In some embodiments, selecting the one or more data analysis tools comprises receiving a user selection of the one or more data analysis tools. In some embodiments, selecting the one or more data analysis tools is automatically performed by the computer without receiving a user selection of the one or more data analysis tools.

In another aspect, the present disclosure provides a computer system for assessing a condition of a subject, comprising: a database that is configured to store a dataset of a biological sample of the subject; and one or more computer processors operatively coupled to the database, wherein the one or more computer processors are individually or collectively programmed to: (i) select one or more data analysis tools comprising: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, a Target Scoring analysis tool, or a combination thereof; (ii) process the dataset using the one or more data analysis tools to generate a data signature of the biological sample of the subject; and (iii) based at least in part on the data signature generated in (ii), assess the condition of the subject.

In another aspect, the present disclosure provides a non-transitory computer readable medium comprising machine-executable code that, upon execution by one or more computer processors, implements a method for assessing a condition of a subject, the method comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject. In any embodiment described herein, the one or more data analysis tools may be a plurality of data analysis tools each independently selected from a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

To obtain a blood sample, various techniques may be used, e.g., a syringe or other vacuum suction device. A blood sample may be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount may vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample may be taken at a first time point and assayed, and then another sample may be taken at a subsequent time point and assayed. Such methods may be used, for example, for longitudinal monitoring purposes to track the development or progression of a disease. In some embodiments, the progression of a disease may be tracked before treatment, after treatment, or during the course of treatment, to determine the treatment's effectiveness.

For example, a method as described herein may be performed on a subject prior to, and after, treatment with a first, second, and/or third disease condition therapy to measure the disease's progression or regression in response to the first, second, and/or third disease condition therapy. The first, second, and/or third disease can be as described above.

After obtaining a sample from the subject, the sample may be processed to generate datasets indicative of a disease or disorder of the subject. For example, a presence, absence, or quantitative assessment of nucleic acid molecules of the sample from a panel of condition-associated genomic loci or nucleotide polymorphism may be indicative of first, second, and/or third disease condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of condition-associated genomic loci. The probes may be nucleic acid primers. The probes may have sequence complementarity with nucleic acid sequences from one or more of the panel of condition-associated genomic loci. The panel of condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more condition-associated genomic loci.

The probes may be nucleic acid molecules (e.g., RNA or DNA) having sequence complementarity with nucleic acid sequences (e.g., RNA or DNA) of one or more genomic loci (e.g., condition-associated genomic loci). These nucleic acid molecules may be primers or enrichment sequences. The assaying of the sample using probes that are selective for the one or more genomic loci (e.g., condition-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., condition-associated genomic loci) to generate the data indicative of the disease or disorder. For example, quantification of array hybridization or polymerase chain reaction (PCR) corresponding to a plurality of genomic loci (e.g., condition-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

The BIG-C (Biologically Informed Gene Clustering) tool may be configured to sort large groups of genes into a set of functional groups (e.g., 53 functional groups). The functional groups are created utilizing publicly available information from online tools and databases including UniProtKB/Swiss-Prot, GO Terms, KEGG pathways, NCBI PubMed, and the Interactome. The functional groups may include one or more of: Active RNA, Anti-apoptosis, anti-proliferation, autophagy, chromatin remodeling, cytoplasm and biochemistry, cytoskeleton, DNA repair, endocytosis, endoplasmic reticulum, endosome and vesicles, fatty acid biosynthesis, cell surface, transcription, glycolysis and gluconeogenesis, golgi, immune cell surface, immune secreted, immune signaling, integrin pathway, interferon stimulated genes, intracellular signaling, lysosome, melanosome, MHC class I, MHC class II, microRNA processing, microRNA, mitochondrial transcription, mitochondria, mitochondria oxidative phosphorylation, mitochondrial TCA cycle, mRNA processing, mRNA splicing, non-coding RNA, nuclear receptor, nucleus and nucleolus, palmitoylation, pattern recognition receptors, peroxisomes, pro-apoptosis, pro-cell cycle, proteasome, pseudogenes, RAS superfamily, reactive oxygen species protection, secreted and extracellular matrix, transcription factors, transporters, transposon control, ubiquitylation and sumoylation, unfolded protein and stress, and unknown. Enrichment scores for each group are calculated based on an overlap p value to determine the functional groups over or under-expressed in the gene expression dataset. The BIG-C may be configured such that each gene is sorted into only one of the 53 functional groups, allowing for a quick and relatively simple understanding of types of genes enriched and co-expressed in a big dataset.

The I-Scope™ tool may be configured to identify immune infiltrates. Hematopoietic cells are unique in that they move throughout the body patrolling for threats to the host, and may infiltrate tissue sites not normally home to immune cells. I-Scope™ may be configured to identify hematopoietic cells through an iterative search of more than 17,000 genes identified in more than 50 microarray datasets. From this search, 1226 candidate genes are identified and researched for restriction in hematopoietic cells as determined by the HPA, GTEx and FANTOM5 datasets (e.g., available at proteinatlas.org). 926 genes meet the criteria for being mainly restricted to hematopoietic lineages (brain, reproductive organ exclusions were permitted). These genes are researched for immune cell specific expression in 27 hematopoietic sub-categories: alpha beta T cell, T cell, regulatory T Cell, activated T cell, anergic T cell, gamma delta T cells, CD8 T, NK/NKT cell, NK cell, T & B cells, B cells, germinal center B cells, B cell and plasmacytoid dendritic cell, T &B & myeloid, B & myeloid, T & myeloid, MHC Class II expressing cell, monocyte, dendritic cell, plasmacytoid dendritic cells, myeloid cell, plasma cell, erythrocyte, neutrophil, low density granulocyte, granulocyte, and platelet. Transcripts are entered into I-Scope™ and the number of transcripts in each category determined. Odd's ratios are calculated with confidence intervals using the Fisher's exact test in R.

The T-Scope™ tool may be configured to help identify types of non-hematopoietic cells in gene expression datasets. T-Scope™ may be configured by downloading approximately 10,000 tissue enriched and 8,000 cell line enriched genes from the human protein atlas along with their tissue or cell line designation (e.g., available at proteinatlas.org). Genes found in more than four tissues are eliminated. Housekeeping genes described in the gene expression study by She et al. are also removed (e.g., as described by She et al., “Definition, conservation and epigenetics of housekeeping and tissue-enriched genes,” BMC Genomics 2009, 10:269, which is incorporated herein by reference in its entirety). This list is further curated by removing genes differentially expressed in 34 hematopoietic cell gene expression datasets and adding kidney specific genes from datasets downloaded from the GEO repository and processed by Ampel BioSolutions. The resulting categories of genes represent genes enriched in the following 42 tissue/cell specific categories: adrenal gland, breast, cartilage, cerebral cortex, uterine cervix, chondrocyte, colon, duodenum, endometrium, epididymis, esophagus fallopian tube, esophagus, fibroblast, heart muscle, keratinocyte, kidney, liver, lung, melanocyte, ovary pancreas, parathyroid gland, placenta, podocyte, prostrate, rectum, salivary gland, seminal vesicle, skeletal muscle, skin, small intestine, smooth muscle, stomach, synoviocyte, testis, kidney loop of henle, kidney proximal tubule, kidney distal tubule, and kidney collecting duct.

The CellScan tool may be a combination of I-Scope™ and T-Scope™, and may be configured to analyse tissues with suspected immune infiltrations that may also have tissue specific genes. CellScan may potentially be more stringent than either I-Scope™ or T-Scope™ because it may be used to distinguish resident tissue cells from non-resident hematopoietic cells.

The MS (Molecular Signature) Scoring tool may be configured to assess specific pathways in a disease state. Information on genes that encode for proteins that participate in a specific signaling pathway, and whether the gene product promotes or inhibits the pathway, are compiled and curated through literature mining. Curated pathways presented by the company include CD40-CD40ligand, IL-6, IL-12/23, TNF, IL-17, IL-21, SIP1, IL-13 and PDE4, but this method may be used for any known signaling pathway with available data. To determine if a signaling pathway is over or under-expressed in a microarray dataset, the gene list for each signaling pathway may be queried against the limma differentially expressed genes from a disease state compared to healthy controls, and the differentially expressed genes in the signaling pathway may be identified for each set. The fold changes for genes that promoted the pathway may be added together and the fold changes for genes that inhibited the pathway may be subtracted from the score. This total score may be normalized based on the number of genes that may be detected on the specific microarray platform used for the experiment. Activation scores of −100 to +100 may be determined using this method with negative scores indicating an inhibition of the specific pathway in the disease state and positive scores indicating an up-regulation of a specific pathway in the disease state. The Fischer's exact test may be performed to determine if there was sufficient overlap of genes between the experimental differentially expressed genes and the genes in the signaling pathway.

Gene Set Variation Analysis (GSVA) may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety). The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

The CoLTs®, or Combined Lupus Treatment Scoring, may be configured to rank identified drugs or therapies by a number of essential characteristics, including scientific rationale, experience in lupus mice/human cells (preclinical), previous clinical experience in autoimmunity, drug properties, and safety profile, including adverse events. Face and test validities may be established by scoring SOC medications and confirming the scores with a panel of lupus clinicians. The final result may be the CoLTs® score. A CoLTs® algorithm may also be configured for drugs in development (DID), which typically do not have drug metabolism and adverse event information available.

The target scoring algorithm may be configured to prioritize a specific gene or protein that is potentially a good choice to target with a drug in first, second and/or third disease patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

### BIG-C™ Big Data Analysis Tool

BIG-CR is a fast and efficient cloud-based tool to functionally categorize gene products. With coverage of over 80% of the genome, BIG-C® leverages publicly available databases such as UniProtKB/Swiss-Prot, GO terms, KEGG pathways, NCBI PubMed and Interactome to place genes into 53 functional categories. The sorting into only one of 53 functional groups allows for a quick and relatively simple understanding of types of genes enriched and co-expressed in a big dataset. This assists in deriving further insights from genes expressed for a given disease state in human or pre-clinical mouse models.

BIG-C® may be used to functionally categorize immunological genes that are not covered in cancer databases such as GO and KEGG (e.g., as described by Grammer et al. 2016, “Drug repositioning in SLE: crowd-sourcing, literature-mining and Big Data analysis,” Lupus, 25 (10), 1150-1170, which is incorporated herein by reference in its entirety). Using a knowledge base of over 5000 patients with systemic lupus erythematosus (SLE), over 16432 genes are each placed into one of 53 BIG-C® functional categories, and statistical analysis is performed to identify enriched categories. BIG-CR categories are cross-examined with the GO and KEGG terms to obtain additional information and insights.

A sample BIG-CR workflow may comprise the following steps. First, SLE genomic datasets are derived from whole blood, peripheral blood mononuclear cells, affected tissues, and purified immune cells. Second, datasets are analyzed using DE analysis (as shown by a differential expression heatmap) or Weighted Gene Coexpression Network Analysis (WGCNA) (as shown by a gene coexpression plot). Third, expressed genes are annotated using publicly available databases (e.g., UniProtKB/Swiss-Prot database, Human Immunodeficiencies database, Mouse MGI database, Entrez Molecular Sequence database, PubMed, and the Human Tissue Atlas). Fourth, signatures are cross-referenced with purified single-cell microarray datasets and RNAseq experiments. Fifth, BIG-CR is leveraged to separate the individual annotated genes into one of 53 functional categories (e.g., as described by Labonte et al. 2018, “Identification of alterations in macrophage activation associated with disease activity in systemic lupus erythematosus,” PloS one, 13 (12), e0208132, which is incorporated herein by reference in its entirety). Sixth, chi-squared analysis is used to determine enriched categories of interest from overlap p-values. Seventh, enriched categories are cross-examined with GO and KEGG terms to derive key insights for further analysis.

### I-Scope™ Big Data Analysis Tool

I-Scope™ may be a tool configured for cross-examining the presence and activity of varying types of immune cell infiltrates with observed gene expression patterns. It may take annotated gene expression data and analyze it for hematopoietic cell lineage. I-Scope™ may be used downstream of the BIG-C® (Biologically Informed Gene-Clustering) tool in that it helps to provide even more insight into the nature of the genes being expressed after categorization.

I-Scope™ addresses the need to understand the involvement of specific cells for a given disease state. While it is helpful to understand the relative up-regulation and down-regulation at the gene expression level, it is even more informative to understand specifically in which cells this is occurring. I-Scope™ may be configured to identify hematopoietic cells through an iterative search of more than 17,000 genes identified in more than 50 microarray datasets (e.g., as described by Hubbard et al., “Analysis of Lupus Synovitis Gene Expression Reveals Dysregulation of Pathogenic Pathways Activated within Infiltrating Immune Cells,” Arthritis Rheumatol, 2018; 70 (suppl 10), which is incorporated herein by reference in its entirety). I-Scope™ may function by restricting the analysis to genes of hematopoietic cell heritage and allow for cross-checking against purified single-cell experiments or datasets. The cross-check confirms and categorizes specific transcript signatures to the 28 hematopoietic cell sub-categories, ultimately allowing for cellular activity analysis across multiple samples and disease states. When combined with BIG-CR categories, the cellular activity may be correlated to specific functions within a given cell type.

A sample I-Scope™ workflow may comprise the following steps. First, candidate genes are identified from SLE (systemic lupus erythematosus) datasets potentially associated with immune cell expression. Second, using HPA, GTEx, and FANTOM5 datasets, expression signatures associated with hematopoietic cell lineage are identified. Third, signatures are cross-referenced with purified single-cell microarray datasets and RNAseq experiments. Fourth, transcripts are categorized into 28 hematopoietic cell sub-categories and assess cellular expression across different samples and disease states. Odd's ratios are calculated with confidence intervals using the Fisher's exact test in R. An I-Scope™ signature analysis for a given sample may lead to the I-Scope™ signature analysis across multiple samples and disease states.

### T-Scope™ Big Data Analysis Tool

The T-Scope™ tool may be configured for cross-examining gene expression signatures of a given sample with a database of non-hematopoietic cell types (e.g., as described by Hubbard et al., “Analysis of Gene Expression from Systemic Lupus Erythematosus Synovium Reveals Unique Pathogenic Mechanisms [Abstract], Annual Meeting of the American College of Rheumatology; June 2019; Chicago, IL, which is incorporated herein by reference in its entirety). T-Scope™ may comprise a database of 704 transcripts allocated to 45 independent categories. Transcripts detected in the sample are matched to one of the cellular categories within the T-Scope™ tool to derive further insights on tissue cell activity. T-Scope™ may be used downstream of the BIG-CR (Biologically Informed Gene-Clustering) tool to understand which tissue cell types are present. In conjunction with I-Scope™ (which provides information related to immune cells), T-Scope™ may be performed to provide a complete view of all possible cell activity in a given sample.

T-Scope™ addresses the need to understand the involvement of specific tissue cells for a given disease state. While it is helpful to understand the relative up-regulation and down-regulation at the gene expression level, it is even more informative to understand specifically in which cells this is occurring. T-Scope™ may be configured by downloading a set of approximately 10,000 tissue enriched and 8,000 cell line enriched genes from the Human Protein Atlas along with their tissue or cell line designation. Genes differentially expressed in hematopoietic cell datasets are removed and kidney specific genes are added from the GEO repository. T-Scope™ may function by restricting the analysis to genes of known tissue cell heritage and allow for cross-checking against purified single-cell experiments or datasets. The cross-check confirms and categorizes specific transcript signatures to the 45 tissue cell sub-categories, ultimately allowing for cellular activity analysis across multiple samples and disease states. When combined with BIG-CR categories, the cellular activity may be correlated to specific functions within a given tissue cell type.

A sample T-Scope™ workflow may comprise the following steps. First, candidate genes are identified from SLE (systemic lupus erythematosus) differential expression datasets potentially associated with tissue cell expression. Second, using publicly available databases, expression signatures associated with potential tissue cell activity are identified. Third, signatures are cross-referenced with microarray, scRNAseq or RNAseq experiments. Fourth, transcripts are categorized into 45 tissue cell sub-categories and cellular expression is assessed across different samples and disease states. Results may be obtained using T-Scope™ in combination with I-Scope™ for identification of cells post-DE-analysis.

### CellScan Big Data Analysis Tool

A cloud-based genomic platform may be configured to provide users with access to CellScan™, which comprises a suite of tools for the identification, analysis, and prioritization of targets for drug development and/or repositioning. This platform is powered by a database containing the genomic information gathered from 5000+ autoimmune patients. The cloud-based genomic platform may leverage results from RNAseq and microarray experiments in conjunction with clinical information, such as medication and lab tests, to provide undiscovered insights.

CellScan™ may go beyond typical ‘omics analysis by performing one or more of the following: functionally categorizing genes and their products (e.g., using BIG-C®); deconvolving gene expression data to identify unique immunological cell types from blood or biopsy samples (e.g., using I-Scope™); identifying tissue specific cell from biopsy samples (e.g., using T-Scope™); identifying receptor-ligand interactions and subsequent signaling pathways (e.g., using MS-Scoring™); ranking genes and their products for targeting by drugs and miRNA mimetics (e.g., using Target-Scoring™); and prioritizing FDA-approved drugs and drugs-in-development for treatment in patients or pre-clinical models (e.g., using CoLTs®).

CellScan™ applications may include one or more of: Biomarker Discovery, Disease Mechanisms, Drug Mechanism of Action, Drug Mechanism of Toxicity, and Target Identification and Validation. Experimental approaches supported by CellScan™ may include one or more of: lncRNA, Metabolomics, MicroArray, miRNA, mRNA, qPCR, Proteomics, and RNAseq.

Data analysis and interpretation with CellScan™ may build on comprehensive, manually curated content of a knowledge base. Powerful, quick, and efficient tools may be used to perform deep analysis of NGS and miRNA data to identify gene function, immunological and tissue cell type, pathways, and target/drug appropriate for a specific disease state.

CellScan™ features may be configured to optimize or maximize the impact of information that surfaces in an analysis so that interpretation of a dataset is comprehensive and elucidates actionable insights. These features may include one or more of: NGS RNAseq data analysis, biomarker scoring, and prioritizing targets and drugs for human clinical trials and/or pre-clinical models. The NGS RNAseq data analysis may comprise interrogating RNA and miRNA data for function, cell-type (immunological or tissue) and pathways. The biomarker scoring may comprise using a knowledge base and gene expression data to assess and prioritize biomarkers associated with a target disease or phenotype. The target/drug prioritization may comprise leveraging objective scoring of targets and drugs based on parameters such as scientific rationale, evidence in mouse/human cells, prior clinical data, overall drug properties, and the risk of adverse events.

The knowledge base may be a repository created from millions of individual pieces of information gathered about genes, cells, tissues, drugs, and diseases, and manually reviewed for accuracy and includes rich contextual details and links to original publications. The knowledge base may enable access to relevant and substantiated knowledge from primary literature as well as public and private databases for comprehensive interpretation of NGS/RNAseq data elucidating function/pathways and prioritize targets/drugs for given disease states. An example list of reference databases for the content in CellScan™, with both human and mouse species-specific identifiers supported.

### MS (Molecular Signature) Scoring™ Analysis Tool

MS-Scoring™ may be configured to identify receptor-ligand interactions and predict ongoing signaling pathways. In addition, MS-Scoring™ may be used to validate molecular pathways as potential targets for new or repurposed drug therapies. The specificity of next-generation drug therapies requires a way to understand the potential of a given therapy to act on the intended biochemical target. Moreover, a potential application of this is the repositioning of drug therapies that may have the correct biochemical targeting to address multiple clinical needs beyond the initial intended therapeutic value.

MS-Scoring™ may be specifically developed to address gaps in the QIAGEN IPA® (Ingenuity Pathway Analysis) tool that does not contain many immunologically relevant pathways. Similar to IPA®, MS-Scoring™ 1 may use log-fold change information to score the target and its signaling pathway to verify the viability of the targets. If the fold-change of the genes of a signaling pathway appears to be upregulated or inhibitors appear to be downregulated, MS-Scoring™ 1 may provide a score of +1. Conversely if the genes of a signaling pathway appear downregulated or the inhibitors upregulated, MS-Scoring™ 1 may provide a score of −1. A score of zero may be provided if no fold-change is observed. The scores may then be summed and normalized across the entire pathway to yield a final % score between-100 (inhibition) and +100 (up-regulation). Higher absolute magnitude scores, scores that are close to −100 or +100, may indicate a high potential for therapeutic targeting. The Fischer's exact test may be performed to determine if there is sufficient overlap of genes between the experimental differentially expressed genes and the genes in the signaling pathway.

A sample MS-Scoring™ 1 workflow may comprise the following steps. First, potential drugs and pathways are identified by LINCS (Library of Integrated Network-Based Cellular Signatures) as candidates for therapeutic intervention. Second, MS-Scoring™ 1 is used to evaluate individual transcript elements of the target pathway. Third, signatures are cross-referenced with purified single-cell microarray datasets and RNAseq experiments. Fourth, scores are compiled and normalized to provide an overall % score for the pathway and higher absolute magnitude scores indicate a higher potential for therapeutic targeting.

MS-Scoring™ 1 may be performed of IL-12 and IL-23 related pathways for targeting using ustekinumab for SLE (systemic lupus erythematosus) drug repositioning (e.g., as described by Grammer et al., 2016, “Drug repositioning in SLE: crowd-sourcing, literature-mining and Big Data analysis,” Lupus, 25 (10), 1150-1170, which is incorporated herein by reference in its entirety).

MS-Scoring™ 2 may utilize custom-defined gene modules that represent a signaling pathway or process and is particularly useful for gene expression datasets from microarray or RNAseq. The MS-Scoring™ 2 tool may be configured to take a deeper look at signaling pathways analyzed using the MS-Scoring™ 1. The tool may analyze raw gene expression data and assess enrichment by the Gene Set Variation Analysis (as described herein), which assigns an indexed score to the individual co-expressed pathways between-1 and +1 indicating levels of down-regulation and up-regulation respectively.

A sample MS-Scoring™ 2 workflow may comprise the following steps. First, a signaling pathway of interest is selected from the MS-Scoring™ 2 menu. Second, a raw gene expression data is inputted into the MS-Scoring™ 2 tool. Third, enrichment of signaling pathway(s) is assessed on a patient by patient basis. Fourth, the data may then be used to drive insight for the target signaling pathways in individual patient samples.

Results from GSVA Analysis on SLE (systemic lupus erythematosus) signaling pathways may be, e.g., as described by Hänzelmann et al., “GSVA: Gene Set Variation Analysis for Microarray and RNA-Seq Data,” BMC Bioinformatics, vol. 14, no. 1, 2013, p. 7., which is incorporated herein by reference in its entirety.

### CoLTs® (Combined Lupus Treatment Scoring) Analysis Tool

A scoring method called CoLTs®, or Combined Lupus Treatment Scoring, may be configured to assessing and prioritizing the repositioning potential of drug therapies. COLTs® may rank identified drugs/therapies by a number of essential characteristics, including scientific rationale, experience in lupus mice/human cells (preclinical), previous clinical experience in autoimmunity, drug properties, and safety profile, including adverse events. Face and test validities may be established by scoring standard of care (SOC) medications and confirming the scores with a panel of lupus clinicians. The final result may be the CoLTs® score. A CoLTs® algorithm may also be configured for drugs in development (DID) since they typically do not have drug metabolism and adverse event information available.

COLTs® may be configured to perform objective scoring of drug molecules based on a hypothesis-based literature search of publicly available databases. The tool has the ability to rank drug molecules from both FDA-approved and non-approved classes and ranked based upon parameters such as scientific rationale, evidence in mouse/human cells, prior clinical data, overall drug properties, and the risk of adverse events. The parameters are used within five independent drug therapy categories: small molecules, biologics, complementary and alternative therapies, and drugs in development.

CoLTs® may address the need for a systematic and objective way to evaluate the potential of drug therapies to be repositioned for treatment of autoimmune diseases, initially within SLE (systemic lupus erythematosus). The composite score may embody all the accessible information in literature databases, inclusive of efficacy and adverse reactions, to be able to assist in the prioritization of drug development. While the composite score takes into account many aspects of a drug, it may heavily weigh the risk of adverse events and ranges from −16 to +11. CoLT Scoring® may be validated through repeated scoring of 215 potential therapies using a total of over 5000 reference data points as well as by clinicians specializing in the field of rheumatology. Specifically, CoLTs®' prediction of Stelara/Ustekinumab to be a top priority biologic for lupus drug repositioning is validated by a successful Phase 2 clinical trial (e.g., as described by Vollenhoven et al., “Efficacy and Safety of Ustekinumab, an IL-12 and IL-23 Inhibitor, in Patients with Active Systemic Lupus Erythematosus: Results of a Multicentre, Double-Blind, Phase 2, Randomised, Controlled Study.” The Lancet, vol. 392, no. 10155, 2018, pp. 1330-1339, which is incorporated herein by reference in its entirety). CoLTs® may be calibrated on SoC (Standard of Care) therapies for the individual autoimmune disease being assessed.

Within the ten major categories, rationale ranges from 0 to +3, mouse/human in vitro experience ranges from −1 to +1, clinical properties are on a scale of −3 to +3, the adverse effect of inducing lupus ranges from −1 to 0, metabolic properties range from −2 to 0, and finally adverse events (such as toxicity, infection, carcinogenic, etc.) were given a score of −5 to 0 (e.g., as described by Grammer et al., 2016, “Drug repositioning in SLE: crowd-sourcing, literature-mining and Big Data analysis,” Lupus, 25 (10), 1150-1170, which is incorporated herein by reference in its entirety). For example, CoLT Scoring® of SOC Therapies in Lupus (Belimumab, HCQ, and Rituximab) may be performed.

### Target Scoring Analysis Tool

The Target scoring algorithm may be configured to prioritize a specific gene or protein that would potentially be a good choice to target with a drug in lupus patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

Target-Scoring™ may be configured to assessing and prioritizing the potential of molecular targets for further development of drug therapies. The Target-Scoring™ tool is very similar to CoLTs® except it approaches the need for new SLE therapies from a different angle. Target Scoring may be configured to perform an objective assessment of molecular targets for the development of new or repurposed drug therapies. Like CoLTs®, it also derives data from a hypothesis-based literature search and generates a composite score based on the publicly available information. Leveraging the composite score, researchers may better prioritize the development of novel drug therapies addressing the assessed targets of interest.

Target-Scoring™ may utilize 19 different scoring categories to derive a composite score that ranges from −13 to +27 for the suitability of a gene target for SLE therapy development. Target-Scoring™ may be validated through repeated scoring of potential therapies as well as by clinicians (e.g., clinicians specializing in the field of immunology).

### Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module may comprise computer systems that connect laboratory hardware or instrumentation with computer systems that process laboratory data. In one embodiment, the data pre-processing module may comprise hardware systems or computer software that performs operations on the data in preparation for analysis. Examples of operations that may be applied to the data in the pre-processing module include affine transformations, denoising operations, data cleaning, reformatting, or subsampling. A data analysis module, which may be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module may use analysis methods, for example, drawn from statistics, mathematics, or biology, to support understanding of the relation between the identified abnormal patterns and health conditions, functional states, prognoses, or risks. A data visualization module may use methods of mathematical modeling, computer graphics, or rendering to create visual representations of data that may facilitate the understanding or interpretation of results.

Feature sets may be generated from datasets obtained using one or more assays of a biological sample obtained or derived from a subject, and a trained algorithm may be used to process one or more of the feature sets to identify or assess a condition (e.g., a disease or disorder, such as first, second, and/or third disease condition) of a subject. For example, the trained algorithm may be used to apply a machine learning classifier to a plurality of condition-associated genomic loci that are associated with two or more classes of individuals inputted into a machine learning model, in order to classify a subject into one of the two or more classes of individuals. For example, the trained algorithm may be used to apply a machine learning classifier to a plurality of condition-associated that are associated with individuals with known conditions (e.g., a disease or disorder, such as first, second, and/or third disease condition) and individuals not having the condition (e.g., healthy individuals, or individuals who do not have first, second, and/or third disease condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as first, second, and/or third disease condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., condition-associated genomic loci) and to produce or output one or more output values based on the plurality of input variables or features (e.g., condition-associated genomic loci). The plurality of input variables or features may comprise one or more datasets indicative of the presence (e.g., positive test result) or absence (e.g., negative test result) of one or more conditions (e.g., a disease or disorder, such as first, second, and/or third disease condition). For example, an input variable or feature may comprise a number of sequences corresponding to or aligning to each of the plurality of condition-associated genomic loci.

The plurality of input variables or features may also include clinical information of a subject, such as health data. For example, the health data of a subject may comprise one or more of: a diagnosis of one or more conditions (e.g., a disease or disorder, such as first, second, and/or third disease condition), a prognosis of one or more conditions (e.g., a disease or disorder, such as first, second, and/or third disease condition), a risk of having one or more conditions (e.g., a disease or disorder, such as first, second, and/or third disease condition), a treatment history of one or more conditions (e.g., a disease or disorder, such as first, second, and/or third disease condition), a history of previous treatment for one or more conditions (e.g., a disease or disorder, such as first, second, and/or third disease condition), a history of prescribed medications, a history of prescribed medical devices, age, height, weight, sex, smoking status, and one or more symptoms of the subject.

For example, the disease or disorder may comprise one or more of: lupus, coronary artery disease (CAD), myocardial infraction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, or glomerulonephritis. As another example, the symptoms may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. As another example, the prescribed medications or drugs may include one or more of: antimalarials, corticosteroids, immunosuppressants, and nonsteroidal anti-inflammatory drugs (NSAIDs).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as first, second, and/or third disease condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as first, second, and/or third disease condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as first, second, and/or third disease condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as first, second, and/or third disease condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as first, second, and/or third disease condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as first, second, and/or third disease condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as first, second, and/or third disease condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as first, second, and/or third disease condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as first, second, and/or third disease condition). In some embodiments, the sample is independent of samples used to train the trained algorithm.

The trained algorithm may be trained with a first number of independent training samples associated with a presence of the condition (e.g., a disease or disorder, such as first, second, and/or third disease condition) and a second number of independent training samples associated with an absence of the condition (e.g., a disease or disorder, such as first, second, and/or third disease condition). The first number of independent training samples associated with presence of the condition (e.g., a disease or disorder, such as first, second, and/or third disease condition) may be no more than the second number of independent training samples associated with absence of the condition (e.g., a disease or disorder, such as first, second, and/or third disease condition). The first number of independent training samples associated with a presence of the condition (e.g., a disease or disorder) may be equal to the second number of independent training samples associated with an absence of the condition (e.g., a disease or disorder, such as first, second, and/or third disease condition). The first number of independent training samples associated with a presence of the condition (e.g., a disease or disorder, such as first, second, and/or third disease condition) may be greater than the second number of independent training samples associated with an absence of the condition (e.g., a disease or disorder, such as first, second, and/or third disease condition).

The trained algorithm may comprise a classifier configured to identify the presence (e.g., positive test result) or absence (e.g., negative test result) of one or more conditions (e.g., a disease or disorder, such as first, second, and/or third disease condition) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as first, second, and/or third disease condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as first, second, and/or third disease condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as first, second, and/or third disease condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as first, second, and/or third disease condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as first, second, and/or third disease condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of condition-associated genomic loci may be identified as most influential or most important to be included for making high-quality classifications or identifications of conditions (or sub-types of conditions). The panel of condition-associated genomic loci, or a subset thereof, may be ranked based on classification metrics indicative of each influence or importance of each individual condition-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of condition-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as first, second, and/or third disease condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of condition-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of condition-associated genomic loci) determined at each of the two or more time points. The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. The assessing may be based at least on the presence, absence, or severity of one or more symptoms, such as alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

In some embodiments, a difference in the feature sets (e.g., quantitative measures of a panel of condition-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of condition-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a prognosis of the condition of the subject.

In some embodiments, a difference in the feature sets (e.g., quantitative measures of a panel of condition-associated genomic loci) determined between the two or more time points may be indicative of the subject having an increased risk of the condition. For example, if the condition was detected in the subject both at an earlier time point and at a later time point, and if the difference is a negative difference (e.g., the quantitative measures of a panel of condition-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of the subject having a decreased risk of the condition. For example, if the condition was detected in the subject both at an earlier time point and at a later time point, and if the difference is a positive difference (e.g., the quantitative measures of a panel of condition-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of condition-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples.

### Kits

The present disclosure provides kits for identifying or monitoring a disease or disorder (e.g., first, second, and/or third disease condition) of a subject. A kit may comprise probes for identifying a quantitative measure (e.g., indicative of a presence, absence, or relative amount) of sequences at each of a panel of condition-associated genomic loci in a sample of the subject. A quantitative measure (e.g., indicative of a presence, absence, or relative amount) of sequences at each of a panel of condition-associated genomic loci in the sample may be indicative of the disease or disorder (e.g., first, second, and/or third disease condition) of the subject. The probes may be selective for the sequences at the panel of condition-associated genomic loci in the sample. A kit may comprise instructions for using the probes to process the sample to generate datasets indicative of a quantitative measure (e.g., indicative of a presence, absence, or relative amount) of sequences at each of the panel of condition-associated genomic loci in a sample of the subject.

The probes in the kit may be selective for the sequences at the panel of condition-associated genomic loci in the sample. The probes in the kit may be configured to selectively enrich nucleic acid (e.g., RNA or DNA) molecules corresponding to the panel of condition-associated genomic loci. The probes in the kit may be nucleic acid primers. The probes in the kit may have sequence complementarity with nucleic acid sequences from one or more of the panel of condition-associated genomic loci. The panel of condition-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct condition-associated genomic loci.

The instructions in the kit may comprise instructions to assay the sample using the probes that are selective for the sequences at the panel of condition-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of condition-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of condition-associated genomic loci in the sample. A quantitative measure (e.g., indicative of a presence, absence, or relative amount) of sequences at each of a panel of condition-associated genomic loci in the sample may be indicative of a disease or disorder (e.g., first, second, and/or third disease condition).

The instructions in the kit may comprise instructions to measure and interpret assay readouts, which may be quantified at one or more of the panel of condition-associated genomic loci to generate the datasets indicative of a quantitative measure (e.g., indicative of a presence, absence, or relative amount) of sequences at each of the panel of condition-associated genomic loci in the sample. For example, quantification of array hybridization or polymerase chain reaction (PCR) corresponding to the panel of condition-associated genomic loci may generate the datasets indicative of a quantitative measure (e.g., indicative of a presence, absence, or relative amount) of sequences at each of the panel of condition-associated genomic loci in the sample. Assay readouts may comprise quantitative PCR (qPCR) values, digital PCR (dPCR) values, digital droplet PCR (ddPCR) values, fluorescence values, etc., or normalized values thereof.

In some embodiments, the dataset comprises RNA gene expression or transcriptome data, DNA genomic data, or a combination thereof. In some embodiments, the biological sample is selected from the group consisting of: a whole blood (WB) sample, a PBMC sample, a tissue sample, and a cell sample. In some embodiments, assessing the SLE condition of the subject comprises determining a diagnosis of the SLE condition, a prognosis of the SLE condition, a susceptibility of the SLE condition, a treatment for the SLE condition, or an efficacy or non-efficacy of a treatment for the SLE condition.

In some embodiments, the method further comprises determining a diagnosis of the SLE condition with a sensitivity of at least about 70%. In some embodiments, the method further comprises determining a diagnosis of the SLE condition with a specificity of at least about 70%. In some embodiments, the method further comprises determining a diagnosis of the SLE condition with a positive predictive value of at least about 70%. In some embodiments, the method further comprises determining a diagnosis of the SLE condition with a negative predictive value of at least about 70%. In some embodiments, the method further comprises determining a diagnosis of the SLE condition with an Area Under Curve (AUC) of at least about 70%. In some embodiments, the method further comprises determining a likelihood of the diagnosis of the SLE condition of the subject.

In some embodiments, the method further comprises generating a plurality of drug candidates for the SLE condition of the subject. In some embodiments, the method further comprises evaluating or predicting a relative efficacy of the plurality of drug candidates for the SLE condition of the subject. In some embodiments, the method further comprises providing a therapeutic intervention comprising one or more of the plurality of drug candidates for the SLE condition of the subject.

In some embodiments, the method further comprises monitoring the SLE condition of the subject, wherein the monitoring comprises assessing the SLE condition of the subject at each of a plurality of time points, and processing the plurality of assessments of the SLE condition of the subject at each of the plurality of time points.

### EXAMPLES

The following illustrative examples are representative of embodiments of the software applications, systems, and methods described herein and are not meant to be limiting in any way.

### Example 1: Genes Causative of Primary Immunodeficiency are Risk Factors for and Over-Expressed in Systemic Lupus Erythematosus

SLE is a chronic, female-biased autoimmune disease defined by the production of high affinity autoantibodies that cause inflammation in many organs, including the skin, kidneys, lungs, central nervous system and hematopoietic system. There is extensive evidence that genetics plays a role in both SLE susceptibility and severity. SLE is characterized by complex genetic inheritance. Genome-wide association studies (GWAS) have identified more than 50 risk loci with ap value<5×10⁻⁸, and these loci identify mechanisms and pathways that may contribute to and/or coincide with disease pathogenesis (1-4). However, these loci are thought to represent less than 30% of disease heritability (5).

Although SLE is commonly thought of as a polygenic disease with modulatory epigenetic features, monogenic lupus has also been reported in a small subset of patients, usually presenting at a very young age (less than 5 years). More than 30 single gene variants have been identified to cause monogenic lupus or a lupus-like phenotype, and these have been important in defining potential pathogenic mechanisms that might contribute to polygenic SLE. Genes underlying monogenic lupus include TREX1, SAMHD1, RAG2, FAS, FASL and various complement components (C1q, C2, C4) (6). Additional rare variants might also contribute to monogenic lupus (7).

An alternative way to identify the genetic basis of a disease such as SLE is the candidate gene approach, in which a specific molecular pathway likely to be associated with the disease is identified from animal models or literature mining and the genes of non-redundant regulators tested for the relationship to disease manifestations. Results from the candidate gene approach have been helpful in identifying specific SLE related genes (8, 9). This approach has been expanded to include networks of genes, such as the so-called immunome (10). Here, this approach was expanded to examine a large series of candidate genes, namely those that have been shown to be causative in human primary immunodeficiency (PID). These genes have been found to play critical roles in development and function of the innate and adaptive immune system, and decreased function results in dramatically increased susceptibility to infection (11, 12). Since the basis of SLE resides in hyperactive immune function, PID genes can be over-represented and also over-expressed in SLE and that varying expression patterns may implicate specific immune pathways involved in lupus pathogenesis.

PID is a series of diseases linked by the genetic predisposition for increased susceptibility to infection with one or more classes of causative infectious organisms. Specific PIDs are characterized by developmental defects or functional inactivation of the adaptive and/or innate immune system, in which the causal genes encode nonredundant steps in controlling infection (13). PIDs are rare, with an incidence of approximately 1 in 1200 births in the United States, but the genes involved clearly indicate a necessary step in human host defense (14). It is noteworthy that a link between PID and autoimmunity has been suggested, since one or more autoimmune or inflammatory conditions was observed in approximately 25% of patients with PID over their lifetime (15). PIDs with coupled autoimmune disorders include B-cell immunodeficiency (XLA, CVID, and Selective IgA deficiency), common immunodeficiency (Wiskott-Aldrich syndrome) and deficiency in early and late complement pathway components. A link between PID and SLE has also been suggested, since complement deficiencies (C1q, C1r, C2, C4, C5, C6, C7, C8, and C9 mutations) frequently present with autoimmune disease and especially SLE (14). In addition, CGD (chronic granulomatous diseases), idiopathic CD4+ lymphocytopenia, and ALPS (autoimmune lymphoproliferative syndrome) present with clinical manifestations of SLE (16).

In the current study, a database of 453 genes found to be causative of PID, was compiled. Notably, a subset of these genes, including STAT4, IRF7, ITGAM, IRAK 4 and TYK2, were predicted to be lupus susceptibility genes from GWAS (17). Here, it was found that 33% of the PID genes are risk genes for SLE. Networks of genes predicted from protein-protein interaction mapping indicated that PID genes largely clustered into categories defining adaptive and innate immune functions. Of the 453 PID genes, as many as 335 (74%) were differentially expressed in lupus samples and of the differentially expressed genes, 280 (61.8%) were over-expressed. PID genes were predominantly overexpressed in patients with active lupus. Using PID defined gene modules as features, machine learning (ML) could successfully classify SLE from healthy controls and active from inactive SLE, although the most important features differed in each classification. The data suggest that many PID genes are risk factors for SLE and that most PID genes are overexpressed in SLE, highlighting the common but opposing nature of the immune and inflammatory pathways essential for host defense on the one hand and immunopathogenesis of SLE on the other.

### Results

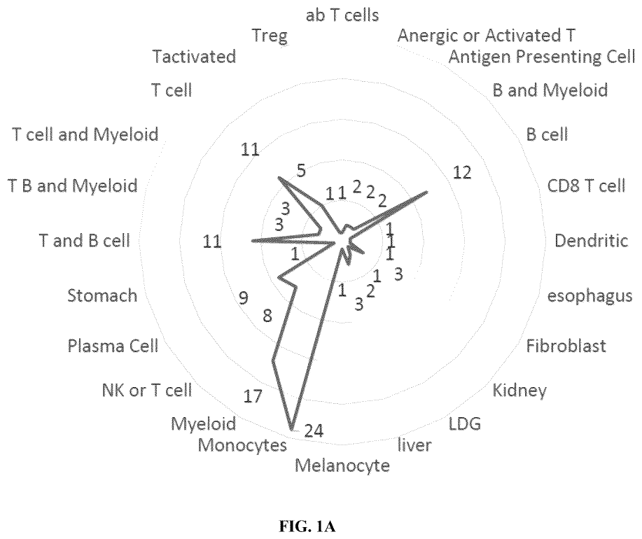

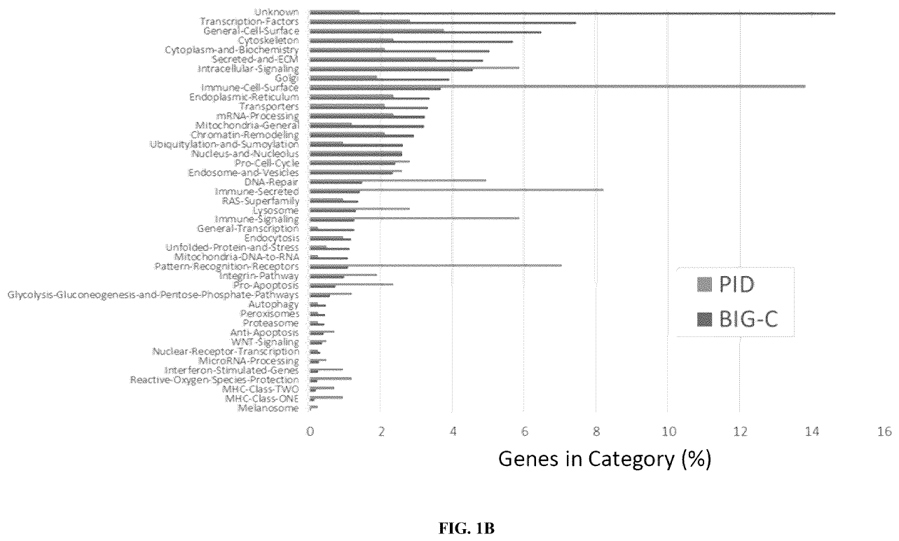

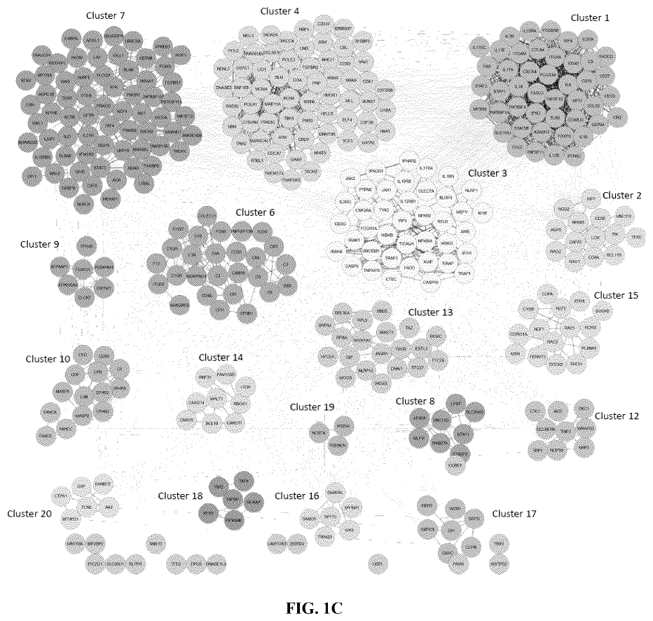

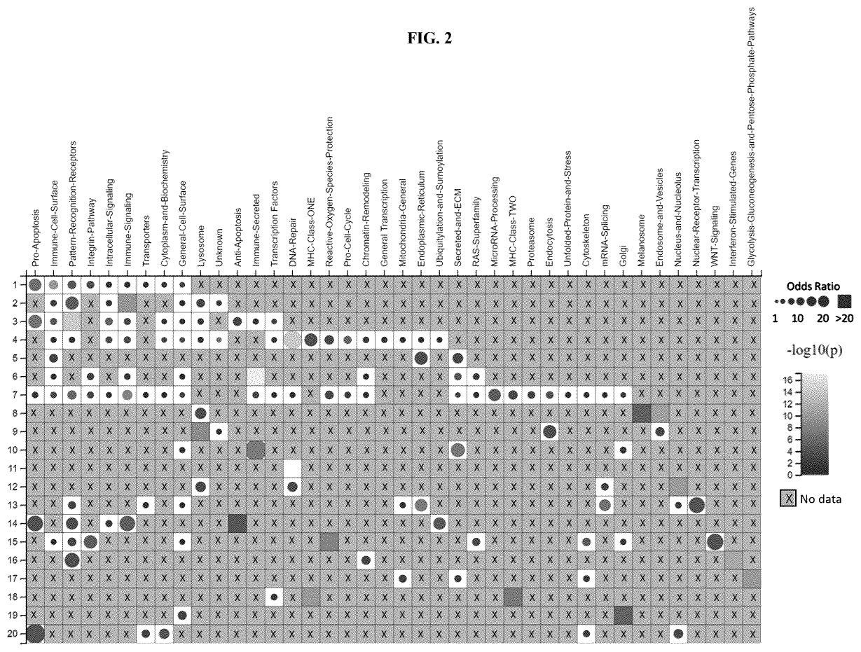

A total of 453 PID genes were compiled into a new database and used for the current analysis (Table 1). To establish the likely cells affected by PID genes, all 453 genes were aggregated and assessed to determine the cells most likely to express them (18). Cell types predicted from the aggregated PID gene database were biased toward immune effector cell types with 125 genes specific for 25 hematopoietic immune cell categories, especially monocytes, myeloid cells, B cell, and T cell lineages (FIG. 1A). Far fewer genes were assigned to tissue-specific categories, with only 1 kidney gene, 3 liver genes, and 1 skin (melanocyte) specific gene. The remaining genes, while not cell-specific enough to signify distinct cell lineages, were nearly all identified as members of immune-related functional categories (Table 2). Analysis of clinical phenotypes associated with each PID gene in the database revealed that of PID genes whose effects could be traced to specific immune cell populations, 64.71% matched the population implicated in this molecular analysis. When limited to only PIDs with identified cellular phenotypes specifically affecting B, T, and PC populations, an 80% overlap with the molecular-based results was observed (data not shown). The most likely functions of the PID genes were also analyzed by assessing their membership in biologic pathways using the Big-C tools. Importantly, the enrichment of PID genes in these specific categories was distinctly different than the categorical distribution of all BIG-C genes themselves, indicating a marked skewing of PID genes toward immune functions (FIG. 1B).

To identify patterns and potential signaling pathways represented by PID genes in greater detail, protein-protein PID interaction networks were generated. The 453 PID genes grouped into 18 mCODE-derived clusters with varying sizes and degrees of interconnectivity (FIG. 1C). The clusters are shown in Table 5, where genes in cluster 1 (FIG. 1C) is listed in Table 5-1, genes in cluster 2 is listed in Table 5-2, genes in cluster 3 is listed in Table 5-3, and the like. Cytoscape produced 18 clusters with the mCODE algorithm even though it annotated 20, but it leaves off clusters that are below a threshold of intra-cluster connectivity or connectivity to the rest of the gene clusters. so even though it assigned numbers to clusters 5 and 11, the mCODE algorithm decided they did not meet the necessary threshold to display, and were not counted in any further analyses.

Functional gene category enrichment indicated that five of the largest clusters (1, 2, 3, 6, and 7) were all dominated by functional molecular categories common to immune cell lineage signatures, including immune cell surface, immune signaling, secreted immune and pattern recognition receptors (PRRs) (FIG. 1C). In addition to immune function, large cluster 7 was enriched in a number of categories representative of general cell function (RAS superfamily, proteasome, cytoskeleton, endocytosis and golgi) as well as processes related to antigen processing (MHC class II) and degradation (proteasome, unfolded protein and stress).

Strikingly, antigen processing was also characteristic of clusters 4 (MHC class I) and 18 (MHC class I and II) highlighting the importance of these pathways for normal immune function. Other notable immune-related clusters included cluster 16, which was strongly enriched specifically for IFN-stimulated genes, and cluster 10, enriched in secreted complement factors (secreted and ECM). The remaining non-immune PID clusters were dominated by a wide range of functions, indicative of metabolism (cluster 17), degradation (clusters 9 and 14) and the cytoskeleton (cluster 15), amongst others. Thus, numerous molecular pathways involved in both immune and general cell functions implicated by the set of PID genes contribute to the diversity of phenotypes observed in PID patients.

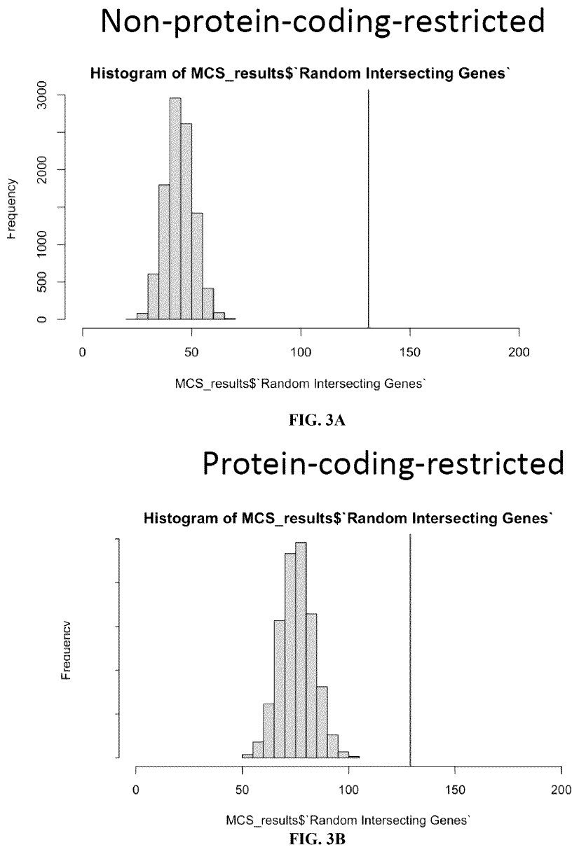

To determine whether PID genes were among the causal genes predicted from SLE associated SNPs, they were matched with a database of individual genes predicted from risk loci identified by multiple large-scale SLE GWAS (Table 3) (17). Out of a total of 453 PID genes, 137 (30%) were SNP-predicted SLE risk genes, including 9 SLE genes (CCL22, CR2, GIF, IFIH1, IRAK1, ITGAM, TNFAIP3, TNFRSF13B and TYK2) in which the SLE SNP resulted in a nonsynonymous amino acid change and 36 SLE genes in which the nucleic acid change occurred in a regulatory region (Table 3). Of the remaining unique SNPs, 85 were noncoding and 7 were long intergenic non-coding RNA (lincRNA) (Table 3). In contrast to the 30% of PID genes overlapping with SNP-predicted SLE genes, only 9.98% of randomly selected genes and 17.8% of randomly selected protein-coding genes overlapped with SNP-predicted SLE risk genes (p<0.0001, FIG. 3A, FIG. 3B).

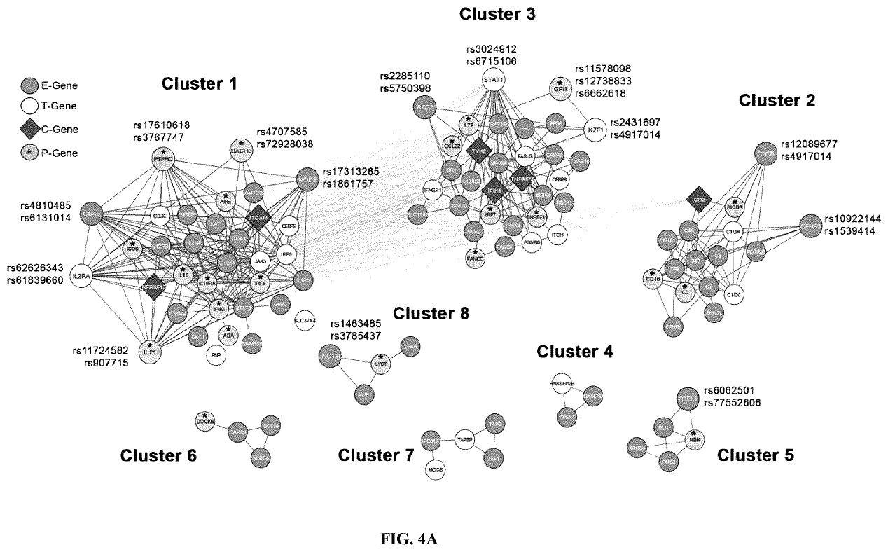

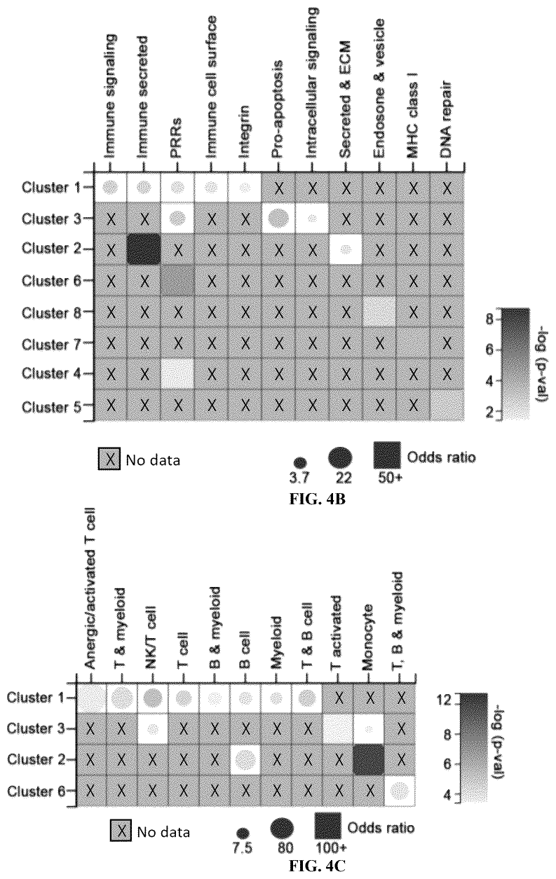

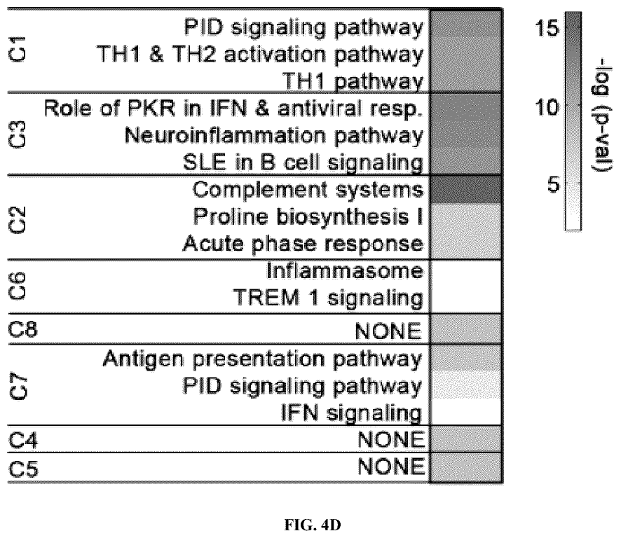

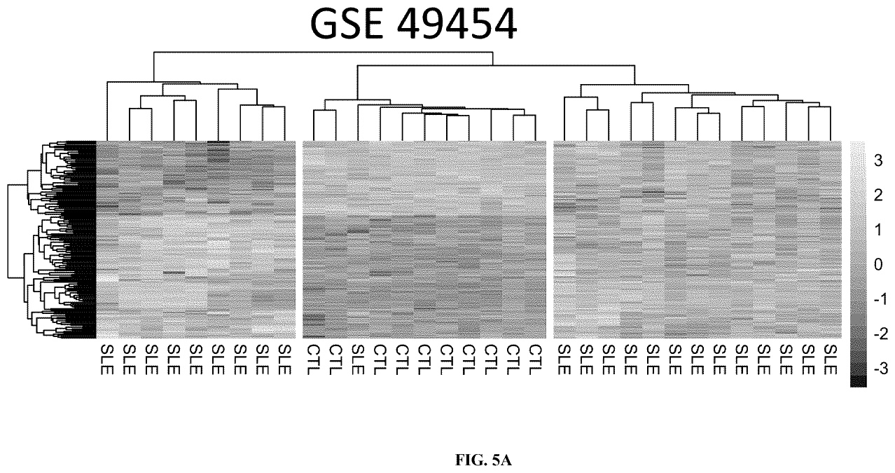

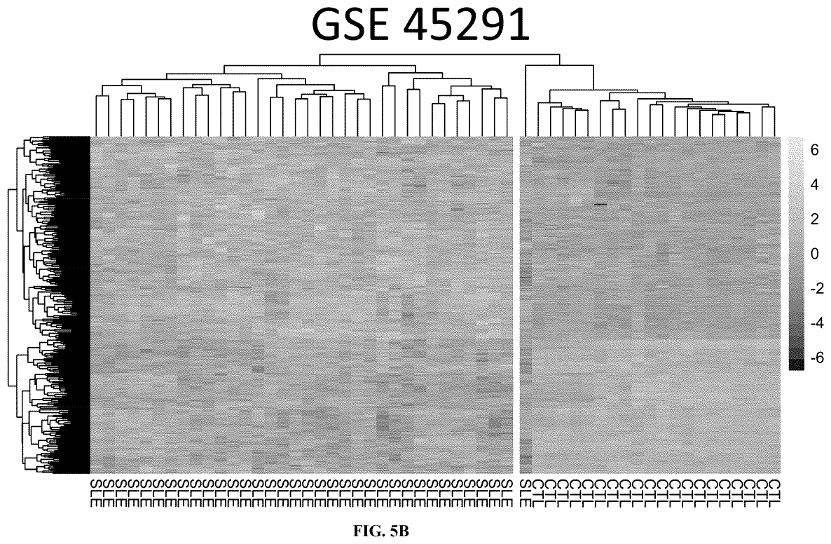

To learn more about the biological relationships among the subset of PID genes that overlapped with SNP-predicted SLE risk genes, overlapping genes were integrated into a protein-protein interaction network and grouped into clusters with the mCODE plugin (FIG. 4A). This method resulted in three larger and five small clusters composed primarily of expression (E-) and proximal (P-) genes. Interestingly, many genes, including CD40, STAT1 and C1QB are implicated by multiple SLE SNPs. Of the three large clusters, cluster 1 contained a high proportion of surface-expressed immune markers and secreted immune factors as well as several pattern recognition receptor genes; cluster 2 contained multiple complement genes; and cluster 3 contained several key intracellular signaling and IFN response genes (FIG. 4A, B). Cell type enrichment analysis showed that cluster 1 was broadly enriched for genes from nearly all immune cell types, whereas cluster 2 was very specifically enriched for monocytes and B cells, and cluster 3 was enriched for NK cells and activated T cells (and to a lesser extent monocytes) (FIG. 4C). IPA canonical pathway analysis confirmed that the key pathways represented in the SLE SNP-predicted gene overlapping with PID genes in clusters 1, 2, and 3 were TH1/TH2 activation pathway, Complement systems, and IFN signaling, respectively (FIG. 4D).

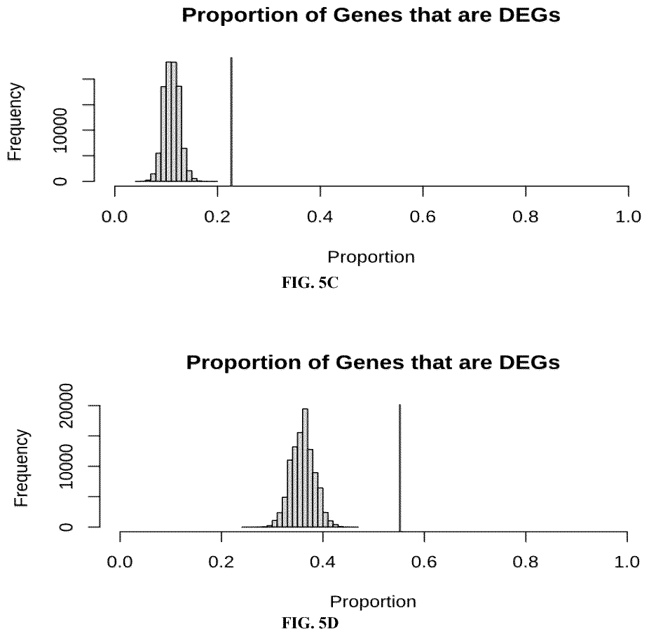

In addition to the striking overlap between PID genes and SLE risk genes predicted by the SNP data, PID genes were also differentially expressed in lupus patients from two independent data sets when compared to healthy controls. By hierarchical clustering based on expression of PID genes, SLE samples clearly were separated from normal in both datasets. In GSE29454, SLE patients divided into two groups, with one cluster exhibiting approximately 60% upregulated and 40% downregulated PID genes and the second cluster of SLE patients exhibiting a more varied picture, but both clearly separated from normal (FIG. 5A). In GSE45291, only one group of SLE samples was noted, which was clearly distinguished from normal, again with about 60% of PID genes overexpressed (FIG. 5B). Overall, the majority of PID genes were consistently overexpressed in the SLE cohorts (FIG. 5A, B) and this was significantly (p<0.0001) greater than expected from random chance as determined by Monte Carlo simulations. (FIG. 5C, D).

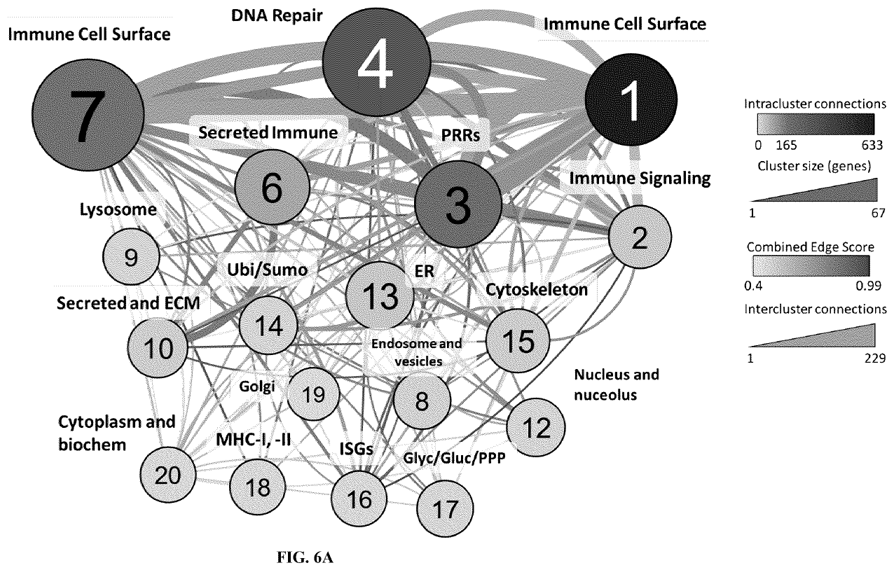

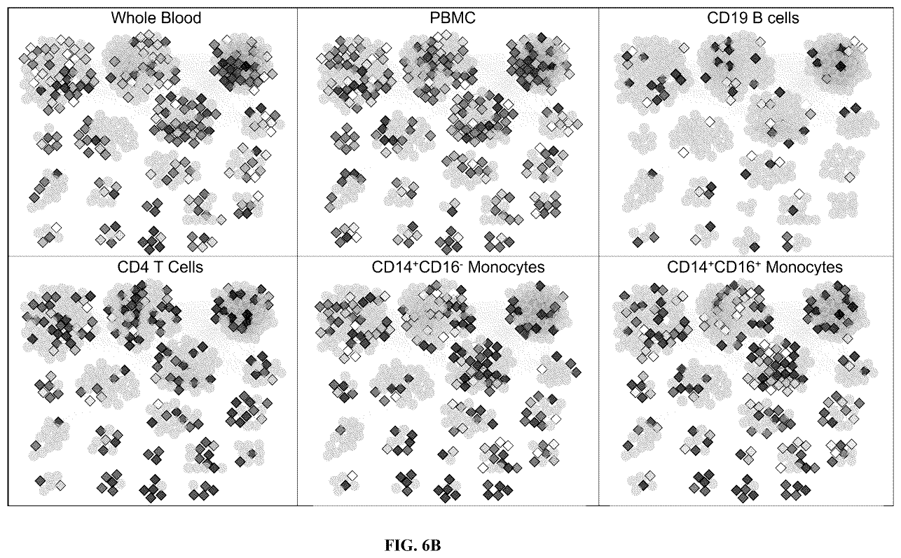

Gene expression data from cell-specific datasets obtained from SLE patients with the PPI network defined by the initial PID gene clusters described in FIG. 1C was combined to determine whether PID genes were differentially expressed between cell populations. Differential expression data from six immune cell datasets were first plotted onto the metastructure of the PPI networks derived from PID gene clusters described in FIG. 1C. (FIG. 6A). As expected, large clusters 1, 2, 3, 7 and 6 were dominated by immune-based processes, whereas many of the remaining smaller clusters were enriched in categories related to general cell function. Next, differentially expressed PID genes were overlayed onto individual gene connectivity maps for each of the six immune cell datasets. The expression patterns between the WB and PBMC datasets seemed broadly conserved, including mixed but generally downregulated PID gene expression in cluster 7, moderate gene upregulation in clusters 1 and 3, and moderate upregulation in several small clusters including 8, 9, 10, 16, and 17 (FIG. 6B, panels 1 (Top left) and 2 (top middle)). Of the two, PBMC displayed slightly enhanced PID upregulation compared to WB in clusters 3, 6, 9, 10, 17, and 20 (FIG. 6B, panel 2). Comparing PID expression patterns of classical (CD14+CD16″) and nonclassical (CD14+CD16+) monocytes revealed that both subpopulations strongly upregulate PID genes across almost all clusters, and nearly identical cluster-based expression patterns suggesting that their expression of PID genes are highly similar (FIG. 6B, panels 5 (bottom middle), 6 (bottom right)). T cells also exhibited a highly positive PID gene expression profile across all clusters (FIG. 6B, panel 4 (bottom left)). Interestingly, cluster 3 was most highly enriched in myeloid cell derived DE genes, whereas cluster 4 was enriched in genes from lymphoid cells (FIG. 6B, panels 3-6).

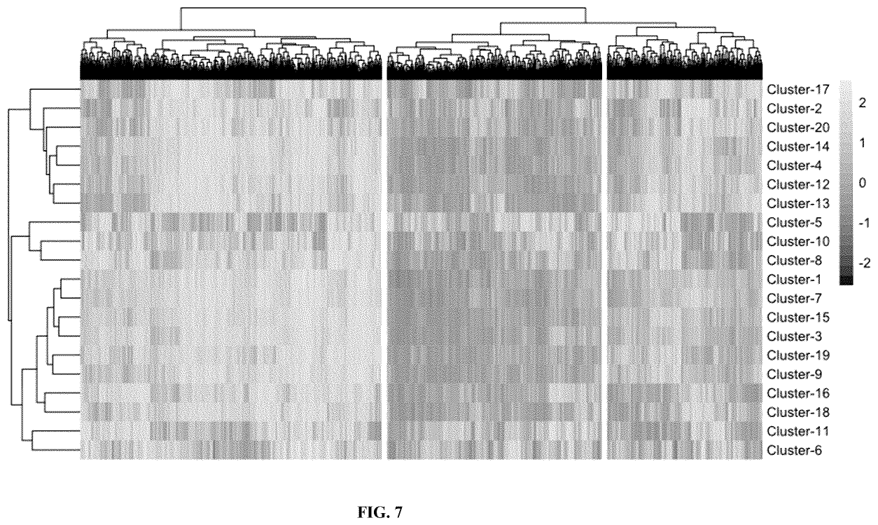

To explore the enrichment of PID genes in lupus patients in greater detail, GSE88884 was interrogated, which contained gene expression data from 1,620 SLE patients. Initially, GSVA was employed using the PID mCODE protein-protein interaction gene modules as the gene test sets. Hierarchical sorting of enrichment values produced three major clades of SLE patients, one with generally high module enrichment, a second with modest enrichment and a third with generally low module enrichment (FIG. 7).

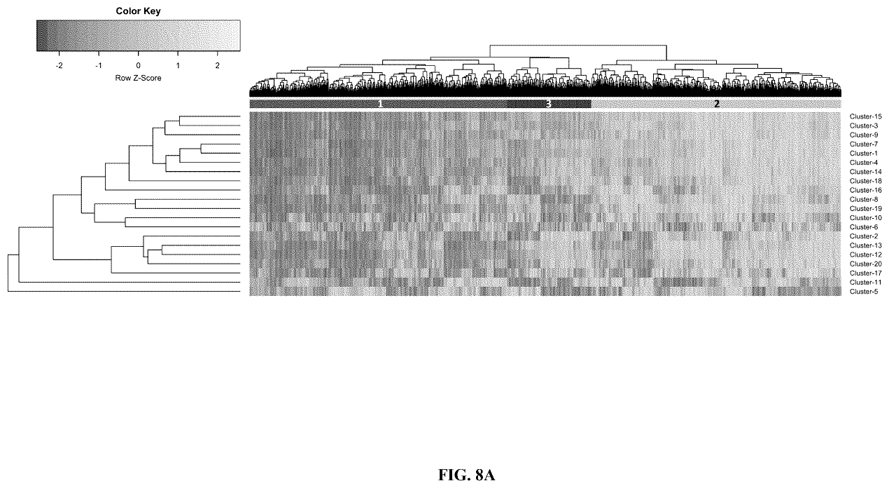

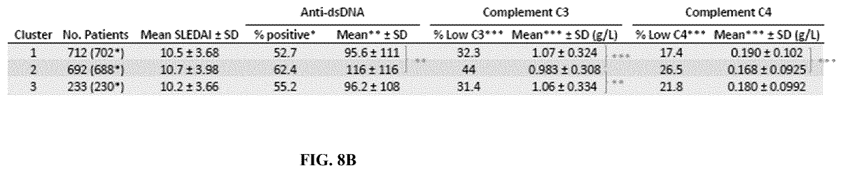

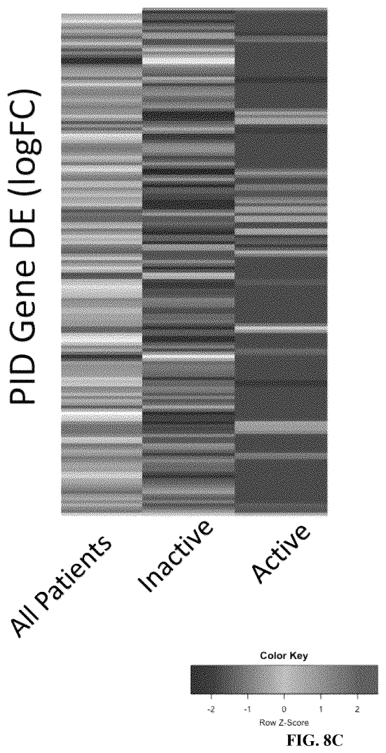

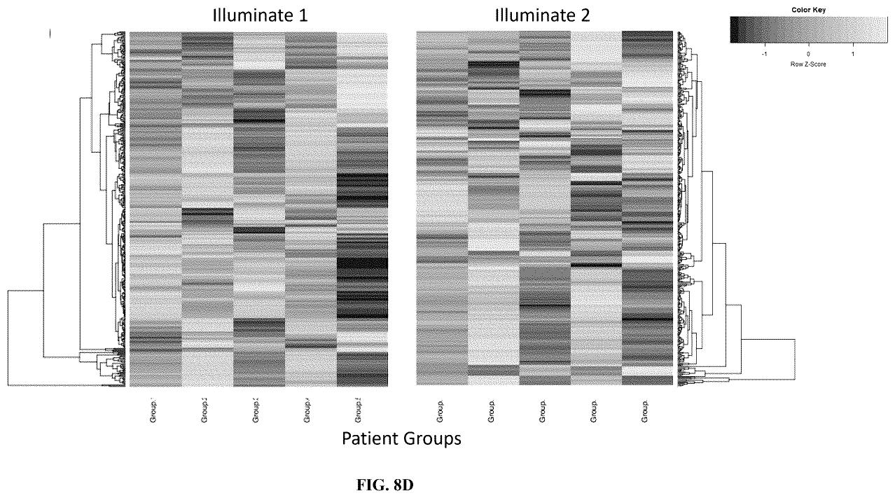

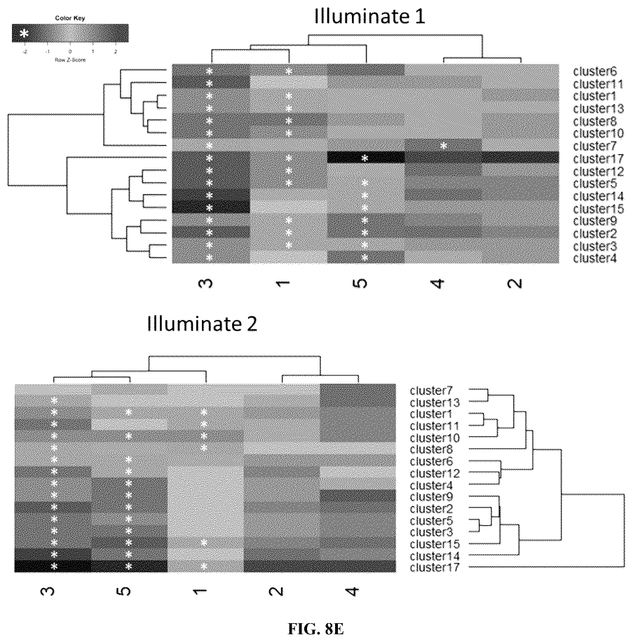

The observation of three apparent PID gene expression groups within the GSE88884 dataset suggested that PID gene expression patterns might be related to disease activity. To test this, the GSE88884 gene expression dataset was clustered based on PID gene module expression, and clinical activity in the SLE patient clusters assessed. (FIG. 8A). Patients grouped into the cohort with high mCODE gene module enrichment (cluster 2) displayed significantly more active disease as indicated by increased anti-dsDNA titers, decreased circulating C3, and decreased circulating C4 (FIG. 8B).

A similar result was obtained from a second dataset (GSE45291) when patients were grouped based upon SLE disease active index (SLEDAI) and then active and inactive patients examined for DE of PID genes (FIG. 8C). To confirm the relationship between SLE disease activity and enrichment of PID gene modules, we grouped patients from GSE88884 based on clinical features using a GMM-VAE and identified 5 phenotypic subsets of patients. As shown in FIG. 8D, differential expression of PID genes was greatest in the most active patient groups (4, 2) compared with the others (1, 2, 3). Similarly, as shown in FIG. 8E, enrichment of PID gene modules determined by GSVA was greatest in Group 2, which exhibited the highest mean SLEDAI (12), the highest incidence of anti-dsDNA (98%), and the highest incidence of low complement (94%). In contrast, Group 3, which contained the least active patients, showed no PID module enrichment, whereas as the other patient clusters showed intermediate PID gene module enrichment (FIG. 8E).

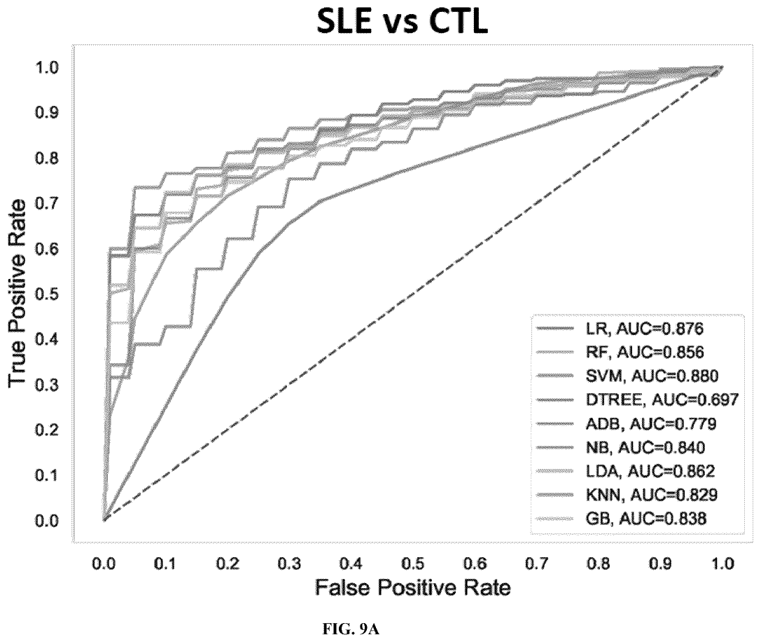

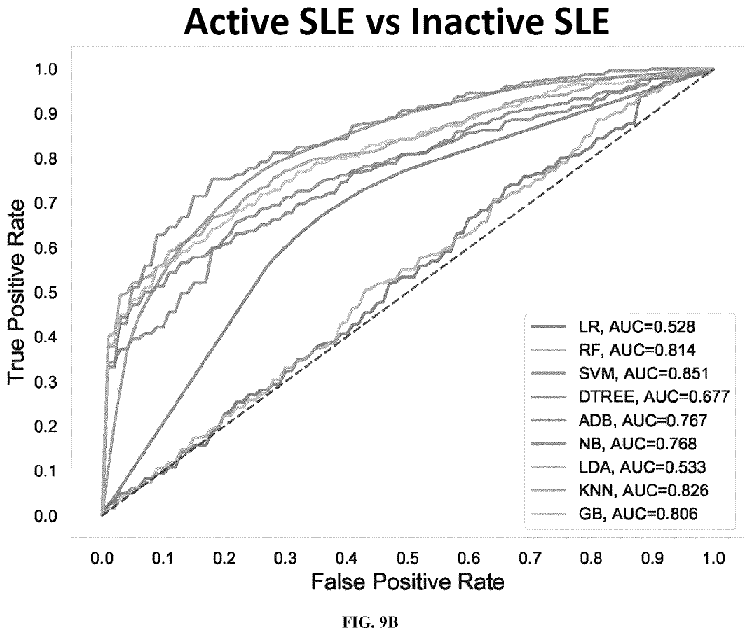

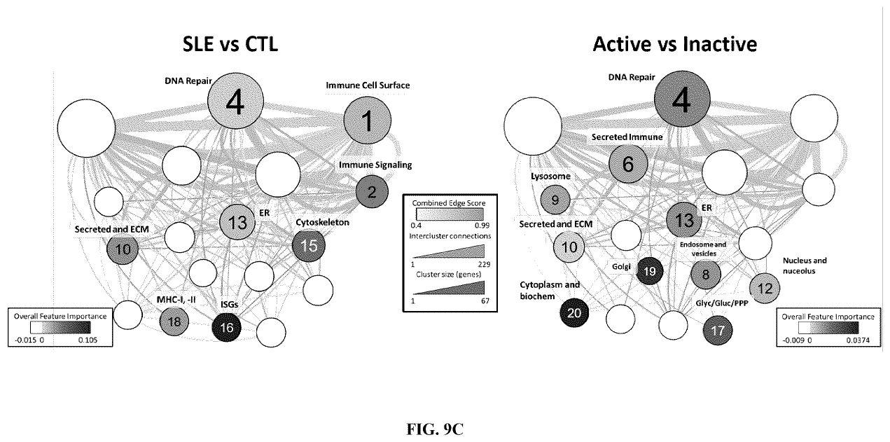

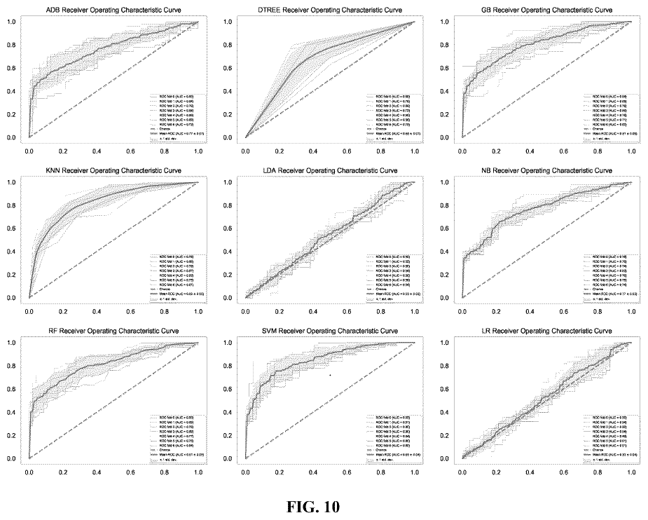

To determine whether enrichment of PID gene expression could be used to classify patients with SLE, combined GSVA scores for enrichment of each of the 18 PID PPI clusters were calculated for five WB datasets and used to train nine machine learning (ML) classifiers (FIG. 9, and FIG. 10). Each ML algorithm attempted to classify subjects based on SLE or control (FIG. 9A) or active or inactive SLE (FIG. 9B) as labels. The ML algorithms effectively classified SLE patients, with the support vector machine (SVM) algorithm achieving the highest accuracy (0.7995) in classifying the SLE cohort from healthy controls (FIG. 9A) and random forest (RF) being the most effective at classifying active versus inactive SLE patients (accuracy of 0.741) (FIG. 9B). Individual classifier performance statistics for the ROC curve of FIG. 9A is presented in Table 4A, and individual classifier performance statistics for the ROC curve of FIG. 9B is presented in Table 4B.

Notably feature importance calculation for each comparison revealed that different combinations of mCODE clusters were the most important for accurate classification in each specific comparison; whereas clusters 16 (ISGs), 15 (cytoskeleton), 18 (MHC-I, -II), and 10 (secreted and ECM) had the highest feature importance in the SLE vs healthy control comparison, prediction of active versus inactive SLE employed clusters 20 (cytoplasm and biochemistry), 19 (Golgi), 4 (DNA repair), and 17 (glycolysis/glucogenesis/pentose phosphate pathway) as the most important (FIG. 9C). Taken together these results demonstrate how distinct combinations of PID gene clusters can be used to distinguish between SLE and normal samples and active versus inactive lupus, respectively.

### DISCUSSION AND CONCLUSION

A novel database of 453 causal PID genes was employed to interrogate patients with SLE and found that these immune response check point genes are disproportionately represented among genes known to increase risk of SLE and genes that are significantly differentially expressed or enriched in SLE. Although some PID conditions have been associated with features of autoimmunity, these results firmly establish that the family of genes whose loss of function is non-redundantly causative of increased susceptibility to infectious agents positively contribute to SLE pathogenesis. Many of these genes overlap with SNP-predicted causative genes of SLE. A majority are overexpressed in SLE compared to healthy donors and even more over-expressed in active SLE. Finally, the association of PID genes with SLE pathogenesis is sufficiently robust that they can be used to classify SLE from healthy controls or active from inactive SLE by ML. Together, these data strongly indicate that the genes identified as being causative of altered host defense to microbial pathogens are primarily involved in the enhanced immune responsiveness underlying SLE.

It was notable that there was a significant overlap between PID genes and risk genes for SLE. eQTL analysis combined with predictions from intergenic enhancer mutations, coding-region variants, and SNP-gene proximity orthogonally identified a group predicted SLE risk genes (4), and a disproportionate percentage of the PID genes were found to overlap with these SNP-predicted SLE risk genes. These findings indicate that genes identified as providing a non-redundant risk for susceptibility to microbial pathogens contribute to the risk of developing SLE. Although the contribution of each of these genes to SLE pathogenesis is small in contrast to their capacity to cause a specific defect in host defense, the results clearly establish a role for these genes in the genetic underpinning of lupus immunopathogenesis.

It is also notable that PID genes are also more likely to be differentially expressed in peripheral blood of SLE patients when compared to random gene cohorts, and this highly significant enrichment holds at multiple levels of filter stringency and across multiple thresholds of Monte Carlo simulation repetition, confirming the hypothesis that PID genes are clearly overexpressed in SLE. Furthermore, SLE-specific expression of PID genes is related to disease activity although enrichment of PID gene expression is also observed in SLE patients with low disease activity. Notably, enrichment of the expression of PID genes in both active and inactive lupus is sufficiently robust to serve as the ML features to classify both SLE from normal and active SLE from inactive disease. The specific patterns of gene expression, however, that serve as the strongest features to discern SLE from control differ from those that classify active versus inactive SLE. PID genes in immune signaling pathways, MHC-related pathways, cytoskeleton pathways, and especially the IGS are uniquely identified as top factors that identify SLE from control. In contrast, mCODE clusters rich in lysosomal, endosomal, metabolic, Golgi-related, and cytoplasm/biochemistry-related PID genes are uniquely defining features between active and inactive SLE. These findings demonstrate that while PID genes are broadly overexpressed in SLE, the nuanced expression patterns they display can be used to track disease progression and activity.

As shown in this work, generation and application of functional gene clusters not only accomplishes the dimensionality reduction required to shrink a large signature into a sufficiently small enough number of deterministically significant genes to be reasonable for diagnostic test development, but also can also identify subgroups of patients that do not appear in bulk differential expression analysis and can better separate patients on the basis of actual clinical features. Whereas a bulk of disease impact is related to the disease activity of the patient, SLEDAI scoring may in fact obscure differences in clinical features/symptoms or exacerbation (19, 20). This hypothesis was tested by applying a variational autoencoder to the 1620 patients from the GSE88884 dataset to evaluate the degree to which disease activity (and the clinical variables from which it is derived) was related to expression of PID genes. The autoencoder sorted SLE patients into five groups based on the presence and/or absence of defined clinical parameters. Notably, PID gene expression appeared to also track with these groups: 89% of PID genes were significantly differentially expressed among the five patient groups, with overexpression of genes in more active patients, decreased expression in somewhat inactive patients, and variable expression in one group potentially related to the presence of lymphopenia. Enrichment of the mCODE gene modules within these patient groups showed similar distributions, i.e., active patient groups assemble together and inactive patient groups assemble together when subjected to hierarchical clustering. This finding reinforces the conclusion that specific, unique combinations of PID genes are directly related to patient outcomes and disease severity.

SLE is a polygenic disease with each non-MHC risk allele contributing a small increase in the chance of developing SLE. It is, therefore, notable that the PID genes convey sufficient risk of developing SLE that they can be used as features in ML to classify the disease from normal and also active from inactive SLE. Although it is known that the confluence of SLE risk alleles can increase the likelihood of developing SLE (21), the contribution of PID genes to SLE risk seems out of proportion to that contributed by random genes or even an aggregate of SLE risk alleles. This is consistent with the conclusion that PID genes encode a unique set of immune check point molecules that disproportionately contribute to SLE risk. Not only does this emphasize the immune nature of SLE risk, but also identifies unique targets that could be employed as novel ways to intervene in this autoimmune disease. Taken together, these results show that PID gene-derived signatures can be used to identify incidence and activity of SLE with very high accuracy. Quantifying PID gene expression and PID gene cluster enrichment may therefore be the basis of focused and directed testing to stratify SLE patients and track disease progression and severity with detail that surpasses current methodology.

Table 1 shows the 453 PID genes.

TABLE 1
453 PID genes listed by Gene Symbol | Entrez ID.

ACD | 65057; ACP5 | 54; ACTB | 60; ADA | 100; ADA2 | 51816; ADAM17 | 6868; ADAR |
103; AICDA | 57379; AIRE | 326; AK2 | 204; AP1S3 | 130340; AP3B1 | 8546; AP3D1 | 8943;
APOL1 | 8542; ARPC1B | 10095; ATM | 472; ATP6AP1 | 537; ATP6V0A2 | 23545; B2M |
567; BACH2 | 60468; BCL10 | 8915; BCL11B | 64919; BLM | 641; BLNK | 29760;
BLOC1S6 | 26258; BTK | 695; C1QA | 712; C1QB | 713; C1QC | 714; CIR | 715; C1S | 716;
C2 | 717; C3 | 718; C4A | 720; C4B | 721; C4BPA | 722; C4BPB | 725; C5 | 727; C6 | 729; C7
| 730; C8A | 731; C8B | 732; C8G | 733; C9 | 735; CARD11 | 84433; CARD14 | 79092;
CARD9 | 64170; CARMIL2 | 146206; CASP10 | 843; CASP8 | 841; CBL | 867; CCBE1 |
147372; CCDC40 | 55036; CCL2 | 6347; CCL22 | 6367; CD19 | 930; CD247 | 919; CD27 |
939; CD3D | 915; CD3E | 916; CD3G | 917; CD40 | 958; CD40LG | 959; CD46 | 4179; CD55
| 1604; CD59 | 966; CD70 | 970; CD79A | 973; CD79B | 974; CD81 | 975; CD8A | 925;
CDCA7 | 83879; CDK4 | 1019; CDK6 | 1021; CEBPE | 1053; CFB | 629; CFD | 1675; CFH |
3075; CFHR1 | 3078; CFHR2 | 3080; CFHR3 | 10878; CFHR4 | 10877; CFHR5 | 81494; CFI |
3426; CFP | 5199; CFTR | 1080; CHD7 | 55636; CIB1 | 10519; CIITA | 4261; CLCN7 | 1186;
CLEC7A | 64581; CLPB | 81570; COLEC11 | 78989; COPA | 1314; CORO1A | 11151; CR2 |
1380; CSF2RA | 1438; CSF2RB | 1439; CSF3R | 1441; CTC1 | 80169; CTLA4 | 1493; CTPS1
| 1503; CTSC | 1075; CXCR4 | 7852; CYBA | 1535; CYBB | 1536; DCLRE1B | 64858;
DCLRE1C | 64421; DKC1 | 1736; DNA2 | 1763; DNAI1 | 27019; DNAJC21 | 134218;
DNASE1L3 | 1776; DNASE2 | 1777; DNMT3B | 1789; DOCK2 | 1794; DOCK8 | 81704;
DSP | 1832; ELANE | 1991; ELF4 | 2000; EPG5 | 57724; ERBIN | 55914; ERCC6L2 |
375748; EXTL3 | 2137; F12 | 2161; FAAP24 | 91442; FADD | 8772; FANCA | 2175; FANCC
| 2176; FANCE | 2178; FAS | 355; FASLG | 356; FAT4 | 79633; FBN1 | 2200; FCGR1A |
2209; FCGR3A | 2214; FCGR3B | 2215; FCN3 | 8547; FERMT3 | 83706; FOXN1 | 8456;
FOXP3 | 50943; FPR1 | 2357; G6PC | 2538; G6PC3 | 92579; G6PD | 2539; GATA2 | 2624;
GFI1 | 2672; GIF | 2694; GINS1 | 9837; GPI | 2821; HAX1 | 10456; HELLS | 3070; HEXB |
3074; HEXIM1 | 10614; HMOX1 | 3162; HOIP1 | 55072; HYOU1 | 10525; ICOS | 29851;
IFIH1 | 64135; IFNAR2 | 3455; IFNG | 3458; IFNGR1 | 3459; IFNGR2 | 3460; IGAD1 |
10986; IGHA1 | 3493; IGHA2 | 3494; IGHE | 3497; IGHG1 | 3500; IGHG2 | 3501; IGHG3 |
3502; IGHG4 | 3503; IGHM | 3507; IGKC | 3514; IGLL1 | 3543; IKBKB | 3551; IKBKG |
8517; IKZF1 | 10320; IL10 | 3586; IL10RA | 3587; IL10RB | 3588; IL12B | 3593; IL12RB1 |
3594; IL12RB2 | 3595; IL17A | 3605; IL17F | 112744; IL17RA | 23765; IL17RC | 84818;
IL18 | 3606; IL1RN | 3557; IL21 | 59067; IL21R | 50615; IL2RA | 3559; IL2RG | 3561;
IL36RN | 26525; IL6 | 3569; IL6ST | 3572; IL7R | 3575; INO80 | 54617; IRAK1 | 3654;
IRAK4 | 51135; IRF2BP2 | 359948; IRF3 | 3661; IRF4 | 3662; IRF7 | 3665; IRF8 | 3394;
ISG15 | 9636; ITCH | 83737; ITGAM | 3684; ITGAX | 3687; ITGB2 | 3689; ITK | 3702;
JAGN1 | 84522; JAK1 | 3716; JAK3 | 3718; KDM6A | 7403; KMT2A | 4297; KMT2D | 8085;
KRAS | 3845; LAMTOR2 | 28956; LAT | 27040; LCK | 3932; LIG1 | 3978; LIG4 | 3981;
LIPA | 3988; LPIN2 | 9663; LRBA | 987; LRRC8A | 56262; LYST | 1130; MAGT1 | 84061;
MALT1 | 10892; MAP3K14 | 9020; MASP1 | 5648; MASP2 | 10747; MBL2 | 4153; MBTPS2
| 51360; MCM4 | 4173; MEFV | 4210; MKL1 | 57591; MLPH | 79083; MOGS | 7841; MPO |
4353; MRE11A | 4361; MS4A1 | 931; MSH6 | 2956; MSN | 4478; MTHFD1 | 4522; MVK |
4598; MX2 | 4600; MYB | 4602; MYD88 | 4615; MYH9 | 4627; MY05A | 4644; MYSM1 |
114803; NBAS | 51594; NBN | 4683; NCF1 | 653361; NCF2 | 4688; NCF4 | 4689; NCSTN |
23385; NEAT1 | 283131; NEIL3 | 55247; NFAT5 | 10725; NFKB1 | 4790; NFKB2 | 4791;
NFKBIA | 4792; NHEJ1 | 79840; NHP2 | 55651; NLRC4 | 58484; NLRP1 | 22861; NLRP12 |
91662; NLRP3 | 114548; NOD2 | 64127; NOP10 | 55505; NRAS | 4893; NSMCE3 | 56160;
ORAI1 | 84876; OSTM1 | 28962; OTULIN | 90268; PARN | 5073; PAX1 | 5075; PCNA |
5111; PEPD | 5184; PEX1 | 5189; PGM3 | 5238; PIEZO1 | 9780; PIGA | 5277; PIK3CD |
5293; PIK3R1 | 5295; PLCG2 | 5336; PLEKHM1 | 9842; PLXNA1 | 5361; PMS2 | 5395; PNP
| 4860; POLA1 | 5422; POLE1 | 5426; POLE2 | 5427; PRF1 | 5551; PRKCD | 5580; PRKDC |
5591; PSEN1 | 5663; PSENEN | 55851; PSMB8 | 5696; PSTPIP1 | 9051; PTEN | 5728;
PTPN6 | 5777; PTPRC | 5788; RAB27A | 5873; RAC1 | 5879; RAC2 | 5880; RAD52 | 5893;
RAG1 | 5896; RAG2 | 5897; RANBP2 | 5903; RASGRP1 | 10125; RASGRP2 | 10235;
RBCK1 | 10616; RECQL4 | 9401; RELB | 5971; RET | 5979; RFX5 | 5993; RFXANK | 8625;
RFXAP | 5994; RHOH | 399; RMRP | 6023; RNASEH2A | 10535; RNASEH2B | 79621;
RNASEH2C | 84153; RNASEL | 6041; RNF168 | 165918; RNF31 | 55072; RNU4ATAC |
100151683; ROR2 | 4920; RORC | 6097; RPL35A | 6165; RPL5 | 6125; RPSA | 3921; RTEL1
| 51750; RUNX1 | 861; SAMD9 | 54809; SAMD9L | 219285; SAMHD1 | 25939; SBDS |
51119; SEC61A1 | 29927; SEMA3E | 9723; SERPING1 | 710; SH2D1A | 4068; SH3BP2 |
6452; SKIV2L | 6499; SLC11A1 | 6556; SLC29A3 | 55315; SLC35C1 | 55343; SLC37A4 |
2542; SLC46A1 | 113235; SMARCAL1 | 50485; SMARCD2 | 6603; SNX10 | 29887; SP110 |
3431; SPINK5 | 11005; SRP54 | 6729; STAT1 | 6772; STAT2 | 6773; STAT3 | 6774;
STAT5B | 6777; STIM1 | 6786; STK4 | 6789; STN1 | 79991; STX11 | 8676; STXBP2 | 6813;
TADA2A | 6871; TAOK2 | 9344; TAP1 | 6890; TAP2 | 6891; TAPBP | 6892; TARBP2 |
6895; TAZ | 6901; TBK1 | 29110; TBX1 | 6899; TCF3 | 6929; TCIRG1 | 10312; TCN2 |
6948; TECR | 9524; TERC | 7012; TERT | 7015; TFRC | 7037; TGFBR1 | 7046; TGFBR2 |
7048; THBD | 7056; TICAM1 | 148022; TINF2 | 26277; TIRAP | 114609; TLR3 | 7098;
TMC6 | 11322; TMC8 | 147138; TMEM173 | 340061; TNFAIP3 | 7128; TNFRSF11A | 8792;
TNFRSF13B | 23495; TNFRSF13C | 115650; TNFRSF1A | 7132; TNFRSF4 | 7293;
TNFSF10 | 8743; TNFSF11 | 8600; TNFSF12 | 8742; TPP1 | 1200; TPP2 | 7174; TRAC |
28755; TRAF3 | 7187; TRAF3IP2 | 10758; TREX1 | 11277; TRIM25 | 7706; TRNT1 | 51095;
TTC37 | 9652; TTC7A | 57217; TYK2 | 7297; UNC119 | 9094; UNC13D | 201294;
UNC93B1 | 81622; UNG | 7374; USB1 | 79650; USP18 | 11274; VAV1 | 7409; VPREB1 |
7441; VPS13B | 157680; VPS45 | 11311; WAS | 7454; WDR1 | 9948; WIPF1 | 7456;
WNT10A | 80326; WRAP53 | 55135; XIAP | 331; XRCC4 | 7518; ZAP70 | 7535; ZBTB24 |
9841; CD4 | 920; P14 | 1029; FCGR2A | 2212; FCGR2B | 2213; FCGRT | 2217; REL | 5966;
STAT5A | 6776; RIPK1 | 8737; ARHGEF1 | 9138; IRF9 | 10379; TWEAK | 51130; CYBC1 |
79415; SPPL2A | 84888; ZNF341 | 84905; LILRA5 | 353514; CAD14 |; TNRT1 |; ARVFC |;
IVNS1ABP | 10625; SOCS1 | 8651;

TABLE 2
BIG-C analysis statistical output and gene breakdown. BIG-C functional categories containing
one or more PID genes are shown and enrichment odds ratios and p-values were calculated
based on number and proportion of genes detected in each as previously described.

| | | | | | # | | |
|---|---|---|---|---|---|---|---|
| BIG-C ® | Odds | | | | Matching | Category | |
| Category | Ratio | p | CF1 | CF2 | genes | size | Matching genes |
| Pattern- | 7.266 | 2.04E−13 | 4.54 | 11.24 | 26 | 196 | CARD9, ERBIN, IFIH1, IRAK1, |
| Recognition- | | | | | | | IRAK4, IRF2BP2, IRF3, IRF7, |
| Receptors | | | | | | | IRF9, ISG15, MEFV, MYD88, |
| | | | | | | | NLRC4, NLRP1, NLRP12, NLRP3, |
| | | | | | | | NOD2, TBK1, TICAM1, TIRAP, |
| | | | | | | | TLR3, TRAF3, TREX1, TRIM25, |
| | | | | | | | UNC93B1, USP18 |
| DNA-Repair | 4.17 | 2.50E−07 | 2.5 | 6.65 | 21 | 255 | CIB1, DCLRE1B, DCLRE1C, |
| | | | | | | | ERCC6L2, FAAP24, FANCA, |
| | | | | | | | FANCC, FANCE, LIG1, LIG4, |
| | | | | | | | MSH6, NBN, NHEJ1, NSMCE3, |
| | | | | | | | PMS2, POLE2, PRKDC, RAD52, |
| | | | | | | | RNF168, RTEL1, XRCC4 |
| Pro- | 3.795 | 0.0006 | 1.76 | 7.32 | 10 | 129 | AK2, BCL10, CASP10, CASP8, |
| Apoptosis | | | | | | | FADD, FAS, FASLG, TNFRSF1A, |
| | | | | | | | TNFSF10, TNFSF12 |
| Interferon- | 3.788 | 0.0262 | 0.98 | 10.46 | 4 | 51 | MX2, SAMD9, SAMD9L, SP110 |
| Stimulated- | | | | | | | |
| Genes | | | | | | | |
| MHC-Class- | 3.49 | 0.0334 | 0.91 | 9.58 | 4 | 55 | B2M, TAP1, TAP2, TAPBP |
| ONE | | | | | | | |
| Reactive- | 2.893 | 0.0945 | 0.57 | 9.08 | 3 | 49 | CYBA, NCF2, NCF4 |
| Oxygen- | | | | | | | |
| Species- | | | | | | | |
| Protection | | | | | | | |
| Melanosome | 2.598 | 0.3323 | 0.06 | 16.68 | 1 | 18 | MLPH |
| Lysosome | 2.508 | 0.005 | 1.26 | 4.54 | 12 | 228 | ACP5, ATP6AP1, ATP6V0A2, |
| | | | | | | | BLOC1S6, CLCN7, CTSC, |
| | | | | | | | DNASE2, HEXB, LIPA, SLC29A3, |
| | | | | | | | TCIRG1, TPP1 |
| MicroRNA- | 2.212 | 0.2387 | 0.26 | 8.6 | 2 | 42 | ADAR, TARBP2 |
| Processing | | | | | | | |
| Glyc/Gluconeo | 2.138 | 0.095 | 0.68 | 5.21 | 5 | 109 | G6PC, G6PC3, G6PD, GPI, |
| and Pentose | | | | | | | SLC37A4 |
| Phosphate | | | | | | | |
| Anti- | 2.077 | 0.1858 | 0.42 | 6.41 | 3 | 67 | CARD14, RIPK1, XIAP |
| Apoptosis | | | | | | | |
| WNT- | 1.473 | 0.4012 | 0.17 | 5.6 | 2 | 62 | ROR2, WNT10A |
| Signaling | | | | | | | |
| Intracellular- | 1.348 | 0.1782 | 0.84 | 2.06 | 24 | 825 | CDCA7, IKBKB, IKBKG, JAK1, |
| Signaling | | | | | | | KRAS, MAP3K14, NFAT5, |
| | | | | | | | NFKB1, NFKB2, NFKBIA, NRAS, |
| | | | | | | | PRKCD, PTEN, REL, RELB, |
| | | | | | | | RNF31, STAT1, STAT2, STAT3, |
| | | | | | | | STAT5A, STAT5B, TAOK2, |
| | | | | | | | TRAF3IP2, TYK2 |
| Pro-Cell- | 1.322 | 0.3112 | 0.67 | 2.37 | 12 | 416 | BLM, CDK4, CDK6, GINS1, |
| Cycle | | | | | | | MCM4, PCNA, POLA1, RECQL4, |
| | | | | | | | RNASEH2A, RNASEH2B, |
| | | | | | | | RNASEH2C, TERT |
| Nucleus- | 1.267 | 0.405 | 0.64 | 2.27 | 12 | 433 | ACD, CTC1, DKC1, DNASE1L3, |
| and- | | | | | | | NOP10, RANBP2, SKIV2L, STN1, |
| Nucleolus | | | | | | | TERC, TINF2, USB1, WRAP53 |
| MHC-Class- | 1.261 | 0.5544 | 0.03 | 7.55 | 1 | 36 | RFXANK |
| TWO | | | | | | | |
| Integrin- | 1.032 | 0.797 | 0.28 | 2.72 | 4 | 175 | ACTB, FBN1, RAC1, RAC2 |
| Pathway | | | | | | | |
| Endosome- | 1.013 | 0.8639 | 0.46 | 1.97 | 9 | 401 | AP1S3, AP3B1, AP3D1, |
| and-Vesicles | | | | | | | LAMTOR2, LYST, PLEKHM1, |
| | | | | | | | RAB27A, SNX10, STX11 |
| Endocytosis | 0.913 | 1 | 0.24 | 2.4 | 4 | 197 | ARPC1B, RET, VPS45, WIPF1 |
| Immune- | 0.862 | 1 | 0.31 | 1.92 | 6 | 312 | C1QB, C1R, C1S, C7, CCL2, |
| Secreted | | | | | | | MASP1 |
| Nuclear- | 0.848 | 1 | 0.02 | 4.97 | 1 | 53 | RORC |
| Receptor- | | | | | | | |
| Transcription | | | | | | | |
| mRNA- | 0.799 | 0.6426 | 0.36 | 1.55 | 9 | 502 | CEBPE, NHP2, PARN, RNASEL, |
| Processing | | | | | | | RPL35A, RPL5, RPSA, SBDS, |
| | | | | | | | TTC37 |
| Chromatin- | 0.731 | 0.5281 | 0.31 | 1.47 | 8 | 486 | CHD7, INO80, KDM6A, KMT2A, |
| Remodeling | | | | | | | KMT2D, MYSM1, SMARCAL1, |
| | | | | | | | SMARCD2 |
| Endoplasmic- | 0.674 | 0.3153 | 0.3 | 1.31 | 9 | 590 | EXTL3, HAX1, HMOX1, JAGN1, |
| Reticulum | | | | | | | PIGA, SEC61A1, SRP54, STIM1, |
| | | | | | | | TECR |
| Secreted- | 0.661 | 0.1872 | 0.35 | 1.15 | 13 | 862 | ADA2, APOL1, CFH, CFHR1, |
| and-ECM | | | | | | | CFHR2, CFHR3, CFHR4, CFHR5, |
| | | | | | | | CFI, COLEC11, FCN3, IL36RN, |
| | | | | | | | PEPD |
| Autophagy | 0.587 | 1 | 0.01 | 3.39 | 1 | 76 | EPG5 |
| General- | 0.57 | 0.0244 | 0.32 | 0.94 | 16 | 1,208 | ADAM17, CD46, CD55, CD59, |
| Cell-Surface | | | | | | | FAT4, FCGRT, IFNAR2, IFNGR1, |
| | | | | | | | IFNGR2, NCSTN, PLXNA1, |
| | | | | | | | SPPL2A, TFRC, TGFBR1, |
| | | | | | | | TGFBR2, TTC7A |
| Peroxisomes | 0.543 | 1 | 0.01 | 3.13 | 1 | 82 | PEX1 |
| Golgi | 0.531 | 0.0773 | 0.23 | 1.07 | 8 | 658 | COPA, LRBA, MASP2, NBAS, |
| | | | | | | | PSEN1, PSENEN, SLC35C1, |
| | | | | | | | VPS13B |
| Transporters | 0.51 | 0.0868 | 0.2 | 1.07 | 7 | 599 | CFTR, MAGT1, ORAI1, PIEZO1, |
| | | | | | | | SLC46A1, TCN2, TMC8 |
| Proteasome | 0.458 | 0.7271 | 0.01 | 2.63 | 1 | 97 | PSMB8 |
| Unfolded- | 0.451 | 0.3325 | 0.05 | 1.67 | 2 | 196 | DNAJC21, MBTPS2 |
| Protein- | | | | | | | |
| and-Stress | | | | | | | |
| Cytoplasm- | 0.431 | 0.0092 | 0.19 | 0.83 | 9 | 895 | CTPS1, LPIN2, MTHFD1, MVK, |
| and- | | | | | | | PGM3, PIK3CD, PIK3R1, PNP, |
| Biochemistry | | | | | | | SAMHD1 |
| Mitochondria- | 0.376 | 0.02 | 0.12 | 0.89 | 5 | 576 | CLPB, DNA2, RMRP, TAZ, UNG |
| General | | | | | | | |
| Cytoskeleton | 0.374 | 0.0013 | 0.17 | 0.72 | 9 | 1,022 | CARMIL2, CORO1A, DOCK2, |
| | | | | | | | DSP, FERMT3, MSN, MYH9, |
| | | | | | | | MYO5A, WDR1 |
| RAS- | 0.356 | 0.1849 | 0.04 | 1.31 | 2 | 247 | ARHGEF1, RASGRP2 |
| Superfamily | | | | | | | |
| Transcription- | 0.327 | 0.0001 | 0.15 | 0.61 | 10 | 1,276 | ELF4, GATA2, HEXIM1, MYB, |
| Factors | | | | | | | PAX1, RFXAP, TBX1, TCF3, |
| | | | | | | | ZBTB24, ZNF341 |
| Immune- | 0.302 | 0 | 0.14 | 0.58 | 9 | 1,242 | CD81, CXCR4, IL10RB, IL17RC, |
| Cell-Surface | | | | | | | IL2RG, IL6ST, IL7R, PTPRC, THBD |
| Ubiquitylation- | 0.296 | 0.0202 | 0.06 | 0.88 | 3 | 440 | ITCH, RBCK1, TPP2 |
| and- | | | | | | | |
| Sumoylation | | | | | | | |
| Immune- | 0.29 | 0.0715 | 0.03 | 1.07 | 2 | 301 | RUNX1, STK4 |
| Signaling | | | | | | | |
| Mitochondria- | 0.262 | 0.1903 | 0.01 | 1.49 | 1 | 168 | TRNT1 |
| DNA-to-RNA | | | | | | | |
| General- | 0.219 | 0.1388 | 0.01 | 1.24 | 1 | 200 | TADA2A |
| Transcription | | | | | | | |
| Unknown | 0.085 | 4.06E−20 | 0.03 | 0.19 | 6 | 2,586 | C17orf62, NEAT1, OSTM1, |
| | | | | | | | SH3BP2, TMC6, UNC119 |

TABLE 3
Overlap between PID gene list and SNP-predicted SLE risk genes.
SNP ID numbers and the associated ancestry groups in which
they were detected are listed for each matching PID gene.

| PID Gene | PID SNPs | Ancestry |
|---|---|---|
| ADA | rs244073 | AsA |
| AICDA | rs4242897 | EA |
| AIRE | rs9784212 | EA |
| AP3D1 | rs12986413 | EA, AA |
| ATP6V0A2 | rs7726414 | EA, AsA |
| BACH2 | rs4707585; rs72928038 | EA; EA |
| BCL10 | rs12726649 | |
| BLM | rs401549 | AsA |
| C1QA | rs12089677 | AsA |
| C1QB | rs12089677; rs4917014 | AsA; EA |
| C1QC | rs12089677 | AsA |
| C2 | rs9268199 | AsA |
| C4A | rs11758312 | AsA |
| C4B | rs11758312 | AsA |
| C5 | rs41308952 | AA |
| C6 | rs112021726 | AsA |
| CARD9 | rs11145974 | EA, AA |
| CASP10 | rs16837131 | EA |
| CASP8 | rs16837131 | EA |
| CCL22 | rs4359426 | AsA |
| CD3E | rs688161 | EA |
| CD40 | rs4810485; rs6131014 | EA; EA |
| CD46 | rs17006749 | AsA |
| CEBPB | rs11117431 | EA |
| CEBPE | rs178770 | AsA |
| CFB | rs3135005 | AsA |
| CFH | rs10922144 | EA, AA |
| CFHR1 | rs10922144 | EA, AA |
| CFHR3 | rs10922144; rs1539414 | EA, AA; EA |
| CFHR4 | rs10922144 | EA, AA |
| CHD7 | rs7011788 | EA |
| COPA | rs1061511 | EA |
| CR2 | rs17617 | EA |
| CTLA4 | rs11571293 | AA |
| DCLRE1B | rs1217397 | AsA |
| DKC1 | rs2728538 | AsA |
| DNMT3B | rs750402 | EA, AA |
| DOCK8 | rs11788947 | EA |
| ERBIN | rs13187024 | AsA |
| FANCC | rs4647361 | AsA |
| FANCE | rs9469857 | AsA |
| FASLG | rs859652 | |
| FCGR3B | rs61803003 | AsA |
| G6PC | rs3785897 | AsA |
| GFI1 | rs11578098; rs12738833; | EA; EA; EA, AA |
| | rs6662618 | |
| GIF | rs35211634 | AsA |
| ICOS | rs10197010 | EA |
| IFIH1 | rs35667974 | EA |
| IFNG | rs11177019 | EA |
| IFNGR1 | rs6932056 | AsA |
| IKBKB | rs6987649 | AsA |
| IKZF1 | rs2431697; rs4917014 | EA, AA, AsA; EA, AsA |
| IL10 | rs3122605 | EA |
| IL10RA | rs2228054 | EA |
| IL12RB1 | rs425648 | EA |
| IL12RB2 | rs10889687 | EA, AA |
| IL1RN | rs7569284 | AsA |
| IL21 | rs11724582; rs907715 | EA; EA |
| IL21R | rs8060368 | AsA |
| IL2RA | rs62626343; rs61839660 | EA; EA |
| IL36RN | rs7569284 | AsA |
| IL7R | rs4869426 | EA |
| INO80 | rs6492983 | EA |
| IRAK4 | rs2407782 | AA |
| IRF4 | rs2431697 | AsA |
| IRF7 | rs2396545 | EA, AA |
| IRF8 | rs11117431 | EA |
| ITCH | rs6088765 | EA |
| ITGAM | rs1143679 | EA, AA |
| ITGAX | rs72799341 | EA, AA |
| JAK3 | rs425648 | EA |
| LAMTOR2 | rs17385169 | AsA |
| LAT | rs7500321 | EA, AA |
| LRBA | rs11946839 | AsA |
| LYST | rs2891663 | EA |
| MLPH | rs13399735 | AsA |
| MOGS | rs6705628 | AsA |
| NBN | rs10504885 | AsA |
| NCF2 | rs41263646 | EA |
| NEAT1 | rs12801636 | EA, AA |
| NFKB1 | rs228614 | AsA |
| NLRC4 | rs13022719 | AsA |
| NOD2 | rs17313265; rs1861757 | AsA; AsA |
| PMS2 | rs10267842 | AsA |
| PNP | rs12588573 | EA |
| PSMB8 | rs17576984 | AsA |
| PTPRC | rs17610618; rs3767747 | EA; AsA |
| RAC2 | rs2285110; rs5750398 | AsA; AsA |
| RBCK1 | rs4815585 | AsA |
| RNASEH2B | rs806321 | AsA |
| RNASEH2C | rs10160596 | AsA |
| ROR2 | rs10991725 | AsA |
| RPSA | rs1768198 | AsA |
| RTEL1 | rs6062501; rs77552606 | AsA; AsA |
| SEC61A1 | rs9872099 | AsA |
| SH3BP2 | rs4690055 | EA, AA, AsA |
| SKIV2L | rs1548306 | AsA |
| SLC11A1 | rs3795985 | AsA |
| SLC29A3 | rs10762485 | AsA |
| SLC37A4 | rs688161 | EA |
| SP110 | rs6756098 | AsA |
| SPINK5 | rs6891892 | AsA |
| STAT1 | rs3024912; rs6715106 | AsA; EA |
| STAT3 | rs2431697 | EA, AA, AsA |
| TAP1 | rs9271366 | AsA |
| TAP2 | rs11758312 | AsA |
| TAPBP | rs2859071 | AsA |
| TAZ | rs3027939; rs5945224 | AsA; AsA |
| TCN2 | rs5753220 | AsA |
| TERT | rs2736100 | AsA |
| TNFAIP3 | rs2230926 | EA, AsA |
| TNFRSF13B | rs3751987 | AsA |
| TNFSF10 | rs9852651 | AsA |
| TRAF3IP2 | rs79074797 | EA, AA |
| TREX1 | rs3135941 | AsA |
| TYK2 | rs34536443 | EA |
| UNC13D | rs1463485; rs3785437 | EA, AA; EA, AA |
| XRCC4 | rs1864174 | AsA |
| ZBTB24 | rs352834 | EA, AA |

TABLE 4A
Individual classifier performance statistics for the ROC curve of FIG. 9A.

| Classifier | Sensitivity | Specificity | Cohen_Kappa_Score | Precision | f-1 score | Accuracy |
|---|---|---|---|---|---|---|
| ADB | 0.7460 | 0.7190 | 0.464162 | 0.7500 | 0.74487 | 0.7335 |
| DTREE | 0.7020 | 0.6975 | 0.397559 | 0.7245 | 0.70936 | 0.6980 |
| GB | 0.7345 | 0.7930 | 0.524047 | 0.8015 | 0.76321 | 0.7640 |
| KNN | 0.7150 | 0.8190 | 0.529521 | 0.8140 | 0.75855 | 0.7655 |
| LDA | 0.7025 | 0.8465 | 0.543056 | 0.8375 | 0.76058 | 0.7710 |
| LR | 0.6890 | 0.8605 | 0.543764 | 0.8480 | 0.75722 | 0.7715 |
| NB | 0.7305 | 0.7980 | 0.525353 | 0.7965 | 0.76006 | 0.7630 |
| RF | 0.7450 | 0.8155 | 0.558135 | 0.8195 | 0.77786 | 0.7815 |
| SVM | 0.7260 | 0.8735 | 0.595964 | 0.8660 | 0.7872 | 0.7995 |

TABLE 4B
Individual classifier performance statistics for the ROC curve of FIG. 9B.

| Classifier | Sensitivity | Specificity | Cohen_Kappa_Score | Precision | f-1 score | Accuracy |
|---|---|---|---|---|---|---|
| ADB | 0.700000 | 0.675714 | 0.376335 | 0.681429 | 0.689773 | 0.687143 |
| DTREE | 0.675714 | 0.630000 | 0.306304 | 0.657857 | 0.657446 | 0.654286 |
| GB | 0.675714 | 0.717143 | 0.393206 | 0.702857 | 0.688140 | 0.697143 |
| KNN | 0.735714 | 0.720000 | 0.455188 | 0.721429 | 0.726782 | 0.727143 |
| LDA | 0.540000 | 0.517143 | 0.058099 | 0.522857 | 0.531035 | 0.531429 |
| LR | 0.505714 | 0.507143 | 0.013168 | 0.502857 | 0.504020 | 0.507143 |
| NB | 0.661429 | 0.712857 | 0.372011 | 0.694286 | 0.676559 | 0.685714 |
| RF | 0.714286 | 0.762857 | 0.479031 | 0.750000 | 0.731173 | 0.741429 |
| SVM | 0.660000 | 0.812857 | 0.473833 | 0.775714 | 0.712113 | 0.735714 |

TABLE 5-1 to 5-20
Gene clusters obtained from clustering 453 PID genes
based on protein-protein interaction networks.

Table 5-1
ADAM17, C3, CCL2, CCL22, CD19, CD27, CD40, CD40LG, CD70, CD79A, CR2,
CTLA4, CXCR4, FAS, FASLG, FCGR3A, FCGR3B, FOXP3, IFNG, IL10, IL10RA,
IL12B, IL17A, IL17F, IL17RC, IL18, IL2RA, IL6, IL7R, IRF8, ITGAM, ITGAX,
MPO, MYD88, PIK3CD, PTPRC, SLC11A1, STAT1, STAT3, STAT5B, TLR3,
TNFRSF1A, TNFRSF4, TNFSF10, TNFSF11
Table 5-2
ACP5, BCL11B, CD3E, CD8A, IRF7, ITK, LCK, NFKB1, NOD2, RAG1, RAG2,
TFRC, UNC119, XIAP, ZAP70
Table 5-3
AIRE, CASP10, CASP8, CD3G, CLEC7A, CSF2RA, CTSC, FADD, FCGR1A,
IFIH1, IFNAR2, IFNGR1, IKBKB, IKBKG, IL10RB, IL12RB1, IL17RA, IL1RN,
IL2RG, IRAK1, IRAK4, IRF3, JAK1, JAK3, MEFV, MVK, NFKB2, NFKBIA,
NLRP1, NLRP3, PGM3, PTPN6, RELB, TICAM1, TIRAP, TNFAIP3, TRAF1,
TRAF3, TYK2, UNC93B1
Table 5-4
ATM, B2M, BLM, CBL, CD247, CD3D, CD81, CDCA7, CHD7, CSF2RB, CSF3R,
CYBA, DCLRE1C, DNA2, DNASE2, DNMT3B, ELF4, ERBB2IP, GATA2, GINS1,
HAX1, HELLS, HMOX1, KRAS, LIG1, LIG4, MCM4, MLL, MRE11A, MSH6,
NBN, NDNL2, NEIL3, NFAT5, NHEJ1, NRAS, OBFC1, PCNA, PMS2, PNP,
POLA1, POLE, POLE2, PRF1, PRKDC, RAD52, RNASEH2A, RNF168, SH3BP2,
SMARCAL1, TADA2A, TAOK2, TBK1, TCF3, TGFBR2, TMEM173, TRAF3IP2,
UNG, VAV1, XRCC4
Table 5-5
CFHR4, PIGA, THBD
Table 5-6
C1QA, C1QB, C1QC, C1R, C1S, C2, C4A, C4BPB, C6, C7, C8A, C8B, C8G, C9,
CD46, CD55, CFH, CFHR1, CFI, COLEC11, F12, FCN3, ICOS, IKZF1, ITGB2,
NCOR2, RASGRP2, SERPING1, SH2D1A, TNFRSF13B
Table 5-7
ACTB, ADA, ADAR, AICDA, APOL1, ARPC1B, BACH2, BLNK, BTK, C4BPA,
CD79B, CEBPB, CFTR, CIB1, CIITA, DNAJC21, ELANE, GFI1, HELLS, IFNGR2,
IGLL1, IL12RB2, IL21, IL21R, IL6ST, INO80, IRF4, ISG15, LAT, LRBA,
LRRC8A, MBL2, MKL1, MS4A1, MYB, MYH9, MYO5A, NCF4, NLRC4, PIK3R1,
PLCG2, PRKCD, PSMB8, PSTPIP1, PTEN, RASGRP1, RET, RNASEH2B,
RNASEH2C, RNASEL, SAMHD1, SMARCD2, STAT2, STK4, TAPBP2, TERT,
TGFBR1, TNFRSF11A, TNFRSF13C, TNFSF12, TREX1, USP18, VPREB1, WAS,
WIPF1
Table 5-8
AP3B1, LYST, MLPH, RAB27A, SLC29A3, STX11, STXBP2, UNC13D
Table 5-9
ATP6AP1, ATP6V0A2, CLCN7, OSTM1, PLEKHM1, TCIRG1, VPS45
Table 5-10
C4B, C5, CD59, CFB, CFD, CFHR2, CFHR3, CFHR5, CFP, MASP1, MASP2
Table 5-11
FANCA, FANCC, FANCE
Table 5-12
ACD, CTC1, DCLRE1B, DKC1, NHP2, NOP10, TINF2, TPP1, WRAP53
Table 5-13
DNAI1, EXTL3, GIF, HYOU1, MAGT1, MOGS, NLRP12, RORC, RPL35A, RPL5,
RPSA, SBDS, SEC61A1, SKIV2L, SRP54, TAZ, TECR, TTC37, TTC7A
Table 5-14
BCL10, CARD11, CARD14, CARD9, FAM105B, ITCH, MALT1, RBCK1, RNF31,
RTEL1, RUNX1
Table 5-15
COPA, CORO1A, CYBB, DOCK2, DOCK8, FERMT3, FPR1, MSN, NCF1, NCF2,
PLXNA1, RAC1, RAC2, RHOH, ROR2
Table 5-16
MX2, MYSM1, SAMD9, SAMD9L, SP110, TRIM25
Table 5-17
CLPB, G6PC, G6PC3, G6PD, GPI, PEPD, WDR1
Table 5-18
RFX5, RFXANK, RFXAP, TAP1, TAP2, TAPBP
Table 5-19
NCSTN, PSEN1, PSENEN
Table 5-20
AK2, CTPS1, DSP, MTHFD1, RANBP2, TCN2

### Methods and Materials

Construction of the PID Gene Database. Monogenic causal PID genes were identified by a thorough search of primary scientific literature, including published studies in PID genetics, PID gene databases, regular reports from PID gene classification panels and gene mutation phenotype databases. Once identified through this mining technique, genes were organized into a database that included the following information for each gene: Gene Symbol, Official Symbol, Full Name, Functional Category (BIG-C, see below), Entrez ID, Ensembl ID, Gene Type, Synonyms, Chromosome Number, Cytogenetic Location, Inheritance, genetic Defect/Pathogenesis, Phenotype, Relevance to SLE, Allelic Mutations (OMIM and Primary literature), Protein Effect (GeneCards), OMIM Gene ID, OMIM Phenotype ID and Mendelian Genetics ID.

Normalization of Raw Data Files. Microarray data (Affymetrix and Illumina): Raw data of each transcriptomic dataset was downloaded from GEO. Statistical analysis was all conducted using R and relevant BioConductor packages. To inspect raw data files for outliers, PCA plots were generated for each dataset. Datasets culled of outliers were cleaned of background noise and normalized using either GCRMA or Robust Multiarray Average (RMA) based on the microarray platform resulting in log 2 transformed expression values into R expression set objects (E-sets). Analysis was conducted using normalized data sets prepared using both standard Affy chip definition file (CDF), as well as custom made BrainArray (BA).

RNA seq data. Raw data files (SRA) were downloaded from NCBI Sequence Read Archive (SRA) website using the SRA toolkit (version 2.10) and converted to FASRQ files using fastq dump. Quality of the FASTQ files was checked using FASTQC software (version 0.11.9). Adapters and poor quality reads were trimmed using Trimmomatic software (Unix based tool version 0.38). Good quality reads were aligned to the human reference genome (hg38) using the STAR aligner (version 2.7). STAR-aligned reads were saved as .sam files and were converted to .bam files using sambamba (version 0.8). Read counts were summarized using featureCounts function of the Subread package (version 1.61). Count normalization and log transformation were carried out using DESeq2 (version 1.32) R package.

Functional annotation of genes via I-SCOPE and T-SCOPE. I-SCOPE is a cellular aggregating tool that categorizes gene transcripts into 32 possible hematopoietic cell categories based on matching 926 transcripts uniquely expressed in hematopoietic cells and known to mark various types of immune/inflammatory cells (22). T-SCOPE is an additional aggregation tool to characterize cell types found in specific tissues in which transcripts are sorted into one of 8 categories representing a specific tissue or tissue cell subtype based on matching 704 total transcripts. Genes in the PID database were cross-referenced with the I-SCOPE and T-SCOPE categories for immune cell and tissue cell types.

Visualization of PID DE and SNP overlap via CIRCOS. CIRCOS diagrams were generated using Circa Genomics Software version 1.2.2. The human hg38 chromosome assembly (GRCh38) was used as a reference and gene base pair coordinates were obtained from the BioMart repository for GRCh38.

Monte Carlo Simulations. Monte Carlo simulations were carried out to determine the probability that a random subset of genes would overlap with DE genes between active lupus patients and controls. The mean of this distribution of outcomes was then compared to the proportion of PID genes that overlapped with DE genes to determine whether expression of PID genes in SLE was more likely than expected from random chance. For each of the two datasets tested (GSE45291 and GSE49454), a random subset of genes equivalent to the number of PID genes present on the respective microarray chip was chosen and sampled 100,000 times using the sample( ) function in R and overlapped with the SLE vs CTL DE genes. The overlaps, or proportions of DEGs, were plotted as a histogram using the hist( ) function in R.

Cross reference to GWAS-defined genes. Monogenic causal PID genes were cross referenced with SLE susceptibility genes derived from the SLE Immunochip GWAS study as described (4). In brief, single nucleotide polymorphisms (SNPs) in high association with SLE incidence were matched to proxy genes via linkage disequilibrium, and corresponding expression quantitiave trait loci (eQTLs) were identified using GTEx v.6 and mapped to their associated expression genes (E-genes). SLE-associated SNPs occurring in transcription factors (T-genes), protein-coding genes (C-genes), or proximal genes (P-genes) were identified via appropriate gene databases (Hacer, GeneHancer, Ensembl genome browser, dbSNP). A total of 5,489 SLE SNP-predicted genes were identified for cross reference with PID genes. Frequency of PID genes identified in this overlap was compared to frequency of overlap with randomly selected sets of genes (451 for all genes on the chip or 427 when limited to protein coding genes) via Monte Carlo simulation with 10,000 iterations as described above.

Protein-protein interaction network construction and cluster creation. Visualization of protein-protein interactions and relationships between genes within datasets was done using Cytoscape (V3.6.0) software and the mCODE StringApp (V1.3.2) plugin application. The Clustermaker2 App (V1.2.1) plugin was used to create clusters of the most related genes within a dataset using a network scoring degree cutoff of 2 and setting a node score cut-off of 0.2, k-Core of 2 and a max depth of 100. DE cell type comparison plots were generated by importing DE values from six datasets (whole blood [WB], GSE39088; peripheral blood mononuclear cells [PBMC], GSE50772; CD14 CD16 classical monocytes, GSE51997; CD14⁺CD16⁺ nonclassical monocytes, GSE51997; CD19+B cells, GSE4588; and CD4+ T cells, GSE51997) as individual node attribute columns and assigning node color to these values with continuous mapping.

Enrichment of functional groups of genes in mCODE-Generated Clusters. Biologically Informed Gene Clustering (BIG-C) is a functional aggregating tool that sorts genes into one of 52 categories based on their most likely biological function and/or cellular localization by utilizing information from multiple online tools and databases (23, 24). Bubble Plots were generated using a custom R-script that simultaneously graphs enrichment odds ratios (circle size) and −log (p) values (circle color).

Compilation of SLE Patient Gene Expression Data. Data were derived from publicly available datasets and collaborators. Raw data files were obtained from the GEO repository for SLE whole blood data. Whole blood-derived datasets GSE45291 and GSE88884 were selected based on the criteria that both SLE patients and healthy controls are included and that both are relatively large in terms of patient number. GSE49454 was also included for additional confirmation and supplementary analyses.

GSE45291 includes 266 female patients (34 active and 232 inactive) and 20 controls. Data for GSE45291 were collected at baseline and include various ancestral backgrounds (Asian, African American, European American, others). Data processing and analysis was conducted using the LIMMA package within the R Suite. Affymetrix CEL files underwent background correction and GCRMA normalization based on annotations using either the onboard Affymetrix chip definition file (CDF) or the hgU133plus2 Enrez Brainarray CDF. Outliers were identified through inspection of the first, second, and third principal components used as axes in a three-dimensional PCA plot, and through inspection of array dendrograms calculated using Euclidean distances and clustered using average/UPGMA agglomeration (unweighted pair group method with arithmetic mean). The LIMMA package was utilized to create linear models of gene expression through empirical Bayesian fitting. The Affymetrix CDF and Brainarray CDF expression sets were analyzed separately. For each, a design matrix was created based on disease state, linear models fitted, and the SLE/normal contrast (expression ratios) extracted for analyses. DE analysis was carried out using moderated t-statistics with related p-values adjusted using Benjamini-Hochberg multiple hypothesis testing. Probes with duplicate gene symbols were removed by retaining the probe with the lowest unadjusted or adjusted p-value, depending on which p-values yielded statistical significance (Affymetrix CDF or Brainarray CDF significant genes were identified using an unadjusted p-value<=0.05 or an adjusted p-value<0.2). The two significant CDF lists were merged and duplicate probes were removed by retaining the most significant probe.

Female patients from GSE88884 were analyzed, including 1620 SLE individuals of various ancestral backgrounds (African American, American Indian, Asian, Pacific Islander, Caucasian, and Mixed) split into two groups, Illuminate 1 and Illuminate 2, derived from study NCT01196091 and study NCT01205438, respectively. Data processing and analysis were conducted as for GSE45291.

Gene Set Variation Analysis (GSVA). The GSVA (V1.25.0) software package for R/Bioconductor was used as a non-parametric, unsupervised method for estimating the variation of pre-defined gene sets in patient and control samples of microarray expression data sets. GSVA was run using GSE88884 and the mCODE clusters. Hedge's G values, a measure of effect size, were calculated from GSVA enrichment scores by contrasting K-S scores of all controls against all lupus patient samples. GSVA enrichment scores were additionally analyzed with Welch's t-tests to identify significant (p<0.05) gene categories contributing to substantial segregation of cohort samples. Results were visualized by using a matrix of Hedge's G values entered as input to a dual scale heatmap 2 function in R. Significant categories are denoted by asterisks.

Evaluation of patient groups by DE-based and GSVA-based hierarchical clustering. DE values of PID genes or GSVA enrichment values of mCODE-derived PID gene clusters for each patient within Illuminate-1 and Illuminate-2 were subjected to hierarchical clustering calculated using Euclidean distances and complete linkage, with k=3 to force establishment of the creation of three patient clusters. For each defined patient group, mean and SD of each of the following clinical traits were calculated: SLE Disease Activity Index (SLEDAI), anti-dsDNA titers (IU), C3 levels (g/L), and C4 levels (g/L). Statistical significance of results from each group was calculated with one-way ANOVA followed by Tukey's test. Significant differences were denoted by asterisks (p_{adj}<0.05, *; p_{adj}<0.01, **; p_{adj}<0.001, ***)

Generation of Illuminate patient groups by Gaussian mixture variational autoencoder (GMVA). Partitioning around medoids (PAM) was used to derive phenotypic-based clusters from the combined patient pool of Illuminate-1 and Illuminate-2 based on clinical trait metadata (SLEDAI, age, alopecia, anti-dsDNA, low complement, ulcers, antimalarial treatment, corticosteroid treatment, immunosuppressant treatment, NSAID treatment, active drug and placebo treatment). A Gaussian mixture variational autoencoder (GMVA) was then trained on clinical data from GSE88884 to identify 5 classes (number of classes chosen by examination of a Bayesian information criterion plot).

Feature Selection Analysis. For the feature selection analysis, the normalized log 2 gene expression matrix (E-set) of each dataset and a database of AMPEL PID gene sets (20) were used as the input. GSVA analysis was run on each dataset separately. Low intensity genes were filtered and only those with IQR>0 across all the samples were considered for GSVA analysis. The GSVA analysis was carried out separately for lupus samples vs normal donors and active lupus samples (SLEDAI>=6) vs inactive lupus samples (SLEDAI<6). GSVA enrichment scores, that range from −1 to +1 from every dataset were concatenated from multiple datasets (GSE88884 ILL-1, GSE88884 ILL-2, GSE45291, GSE39088, & GSE112087), providing a sufficiently large cohort for feature extraction and to stratify lupus patients based on disease activity.

ML Techniques: Various feature selection techniques were employed to remove the noise and select features which contribute most to the prediction variable. The concatenated GSVA score matrix was used as input. The analysis was carried out as follows:

Two GSVA concatenated matrices were created and designated as 1: Discovery cohort1—1936 lupus samples 96 normal donors (GSE88884 ILL-1, GSE88884 ILL-2, GSE45291, GSE39088, GSE112087); and 2: Discovery cohort2-1665 active lupus samples 242 inactive lupus samples (GSE88884 ILL-1, GSE88884 ILL-2, GSE45291, GSE39088, GSE112087). Feature extraction analysis was carried out in Python using scikit-learn (version 0.24.1) independently on discovery cohort1 and discovery cohort2 and involved removing missing features and any features with low variance across all samples of each cohort.

Two independent binary classifications of discovery cohort1 and discovery cohort2 were carried out using scikit-learn (version 0.24.1) library in Python (version 3.8.2). Several linear, nonlinear, and ensemble ML algorithms such as Logistic Regression (LR), K-Nearest Neighbor (KNN), Naïve Bayes (NB), Support Vector Machine (SVM), Random Forest (RF), Gradient Boosting (GB), Decision Tree (DTREE), Linear Discriminant Analysis (LDA), and Adaptive Boosting (ADB) were implemented to distinguish lupus from normal donors and active lupus samples from inactive lupus samples. The performance of various binary classifiers was evaluated based on sensitivity, specificity, Cohen kappa score, f1-score, and accuracy. Because of imbalances in the number of SLE and normal samples in the cohorts, sub-sampling without replacement was employed by creating 20 different folds/subsets by randomly selecting 96 lupus samples to match with the minority class (normal) in discovery cohort1 and 7 folds/subsets by randomly selecting 242 active lupus samples to match with the minority class (inactive lupus) in cohort 2. The data from each fold of discovery cohort1 was split into 70% training and 30% of validation. Various ML classifiers were built on training data and evaluated on validation data. Average performance measures, including sensitivity, specificity, accuracy, f1-score and Cohen kappa score, were calculated from all 20 different folds of discovery cohort1 and 7 different folds of discovery cohort2. Receiver Operating Characteristic (ROC) curves and Precision-Recall (PR) curves were plotted using the matplotlib (Version 3.3.4) library of Python. The permutation importance function from SVM was used to calculate the feature importance score to identify the top predictors that classify lupus samples from normal donors or active lupus from inactive lupus samples.

### REFERENCES

1. Bentham, J. et al. Genetic association analyses implicate aberrant regulation of innate and adaptive immunity genes in the pathogenesis of systemic lupus erythematosus. Nat. Genet. 47, 1457-1464 (2015).
2. Graham, R. R., Hom, G., Ortmann, W. & Behrens, T. W. Review of recent genome-wide association scans in lupus. in Journal of Internal Medicine 265, 680-688 (J Intern Med, 2009).
3. Yang, W. et al. Genome-wide association study in asian populations identifies variants in ETS1 and WDFY4 associated with systemic lupus erythematosus. PLoS Genet. 6, (2010).
4. Owen, K. A. et al. Analysis of Trans-Ancestral SLE Risk Loci Identifies Unique Biologic Networks and Drug Targets in African and European Ancestries. Am. J. Hum. Genet. 107, 864-881 (2020).
5. Julià, A. et al. Genome-wide association study meta-analysis identifies five new loci for systemic lupus erythematosus. Arthritis Res. Ther. 20, (2018).
6. Alperin, J. M., Ortiz-Fernández, L. & Sawalha, A. H. Monogenic Lupus: A Developing Paradigm of Disease. Frontiers in immunology 9, 2496 (2018).
7. Demirkaya, E., Sahin, S., Romano, M., Zhou, Q. & Aksentijevich, I. New Horizons in the Genetic Etiology of Systemic Lupus Erythematosus and Lupus-Like Disease: Monogenic Lupus and Beyond. J. Clin. Med. 9, 712 (2020).
8. Cunninghame Graham, D. S. & Vyse, T. J. The candidate gene approach: Have murine models informed the study of human SLE? Clinical and Experimental Immunology 137, 1-7 (2004).
9. Sandling, J. K. et al. A candidate gene study of the type i interferon pathway implicates IKBKE and IL8 as risk loci for SLE. Eur. J. Hum. Genet. 19, 479-484 (2011).
10. Siddani, B. R., Pochineni, L. P. & Palanisamy, M. Candidate Gene Identification for Systemic Lupus Erythematosus Using Network Centrality Measures and Gene Ontology. PLoS One 8, e81766 (2013).
11. Bucciol, G. et al. Lessons learned from the study of human inborn errors of innate immunity. J. Allergy Clin. Immunol. 143, 507-527 (2019).
12. Tangye, S. G. et al. Human Inborn Errors of Immunity: 2019 Update on the Classification from the International Union of Immunological Societies Expert Committee. J. Clin. Immunol. 40, 24-64 (2020).
13. Schmidt, R. E., Grimbacher, B. & Witte, T. Autoimmunity and primary immunodeficiency: Two sides of the same coin? Nature Reviews Rheumatology 14, 7-18 (2018).
14. McCusker, C. & Warrington, R. Primary immunodeficiency. Allergy, Asthma Clin. Immunol. 7, (2011).
15. Thaventhiran, J. E. D. et al. Whole-genome sequencing of a sporadic primary immunodeficiency cohort. Nature 583, 90-95 (2020).
16. Errante, P. R., Perazzio, S. F., Frazão, J. B., Da Silva, N. P. & Andrade, L. E. C. Primary immunodeficiency association with systemic lupus erythematosus: Review of literature and lessons learned by the Rheumatology Division of a tertiary university hospital at São Paulo, Brazil. Revista Brasileira de Reumatologia 56, 58-68 (2016).
17. Owen, K. A. et al. Analysis of Trans-Ancestral SLE Risk Loci Identifies Unique Biologic Networks and Drug Targets in African and European Ancestries. Am. J. Hum. Genet. 107, 864-881 (2020).
18. Hubbard, E. L. et al. Analysis of gene expression from systemic lupus erythematosus synovium reveals myeloid cell-driven pathogenesis of lupus arthritis. Sci. Rep. 10, (2020).
19. Lattanzi, B. et al. Measures of disease activity and damage in pediatric systemic lupus erythematosus: British Isles Lupus Assessment Group (BILAG), European Consensus Lupus Activity Measurement (ECLAM), Systemic Lupus Activity Measure (SLAM), Systemic Lupus Erythematosus Disease Activity Index (SLEDAI), Physician's Global Assessment of Disease Activity (MD Global), and Systemic Lupus International Collaborating. Arthritis Care Res. 63, (2011).
20. Zayat, A. S. et al. Defining inflammatory musculoskeletal manifestations in systemic lupus erythematosus. Rheumatology 58, 304-312 (2019).
21. Langefeld, C. D. et al. Transancestral mapping and genetic load in systemic lupus erythematosus. Nat. Commun. 8, (2017).
22. Ren, J. et al. Selective histone deacetylase 6 inhibition normalizes b cell activation and germinal center formation in a model of systemic lupus erythematosus. Front. Immunol. 10, 2512 (2019).
23. Catalina, M. D., Bachali, P., Geraci, N. S., Grammer, A. C. & Lipsky, P. E. Gene expression analysis delineates the potential roles of multiple interferons in systemic lupus erythematosus. Commun. Biol. 2, (2019).
24. Catalina, M. D., Owen, K. A., Labonte, A. C., Grammer, A. C. & Lipsky, P. E. The pathogenesis of systemic lupus erythematosus: Harnessing big data to understand the molecular basis of lupus. J. Autoimmun. 110, (2020).

While preferred embodiments have been shown and described herein, it will be obvious to those skilled in the art that such embodiments are provided by way of example only. Numerous variations, changes, and substitutions will now occur to those skilled in the art without departing from the scope of the disclosure. It may be understood that various alternatives to the embodiments described herein may be employed in practice. Numerous different combinations of embodiments described herein are possible, and such combinations are considered part of the present disclosure. In addition, all features discussed in connection with any one embodiment herein may be readily adapted for use in other embodiments herein. It is intended that the following claims define the scope of the disclosure and that methods and structures within the scope of these claims and their equivalents be covered thereby.

1-47. (canceled)

48. A method for determining whether a subject has systemic lupus erythematosus (SLE), the method comprising:
(a) providing expression data of a plurality of circulating blood ribonucleic acid (RNA) markers from the subject, wherein the expression data comprises expression data for at least 10 markers selected from any of Table 3, and Tables 5-1 to 5-20; and
(b) analyzing the expression data to determine whether the subject has SLE with an area under the curve (AUC) of at least 0.65.

49. The method of claim 48, wherein the expression data comprises expression data for at least 20 markers selected from any of Table 3, and Tables 5-1 to 5-20.

50. The method of claim 48, wherein the expression data comprises expression data for at least 50 markers selected from any of Table 3, and Tables 5-1 to 5-20.

51. The method of claim 48, wherein the plurality of circulating blood RNA markers comprise cell-free RNA markers.

52. The method of claim 48, wherein the plurality of circulating blood RNA markers comprise intracellular RNA markers.

53. The method of claim 48, further comprising, before (a), obtaining a sample from the subject comprising the plurality of circulating blood RNA markers.

54. The method of claim 52, wherein the sample comprises a blood sample.

55. The method of claim 52, wherein the sample comprises peripheral blood mononuclear cells (PBMCs).

56. The method of claim 52, wherein the sample comprises a tissue sample.

57. The method of claim 52, further comprising sequencing the plurality of circulating blood RNA markers to obtain the expression data.

58. The method of claim 48, wherein the AUC is at least 0.7.

59. The method of claim 48, wherein the AUC is at least 0.8.

60. The method of claim 48, wherein the at least 10 markers selected from any of Table 3, and Tables 5-1 to 5-20 comprise at least 3 markers comprising one or more single nucleotide polymorphisms (SNPs).

61. The method of claim 48, wherein (b) comprises performing a gene set variation analysis (GSVA).

62. The method of claim 48, wherein (b) comprises performing a multiscale embedded gene co-expression network analysis (MEGENA).

63. The method of claim 48, further comprising, after (b), outputting a report indicating whether the subject has SLE.

64. The method of claim 48, further comprising, after (b), administering to the subject a drug that treats SLE.

65. The method of claim 64, wherein the drug an antimalarial, a corticosteroid, an immunosuppressant, a nonsteroidal anti-inflammatory drug (NSAIDs), a biologic, or any combination thereof.

66. The method of claim 48, wherein the analyzing further comprises a specificity of at least 70%.

67. The method of claim 48, wherein the analyzing further comprises a sensitivity of at least 70%.
